SEQUENCE LISTING (10 sequences)

SEQ ID NO: 1 - protein, 14 aa, Artificial Sequence
  Description of Artificial Sequence Synthetic peptide
  MOD_RES (6)..(6): Ala or Ser
GSTSGXGKSSEGKG

SEQ ID NO: 2 - protein, 18 aa, Artificial Sequence
  Description of Artificial Sequence Synthetic peptide
GSTSGSGKPGSGEGSTKG

SEQ ID NO: 3 - protein, 7 aa, Artificial Sequence
  Description of Artificial Sequence Synthetic peptide
GGGGGGS

SEQ ID NO: 4 - protein, 15 aa, Artificial Sequence
  Description of Artificial Sequence Synthetic peptide
GGGGSGGGGSGGGGS

SEQ ID NO: 5 - DNA, 1611 bp, Artificial Sequence
  Description of Artificial Sequence Synthetic polynucleotide
atgtgccatcagcaacttgtcatctcctggttctccctcgtgttcctggcctcccctcttgtcgcgatttgggagctgaagaaagatgtgtacgtcgtggaactcgactggtacccggacgcccccggggaaatggtggtgctcacttgtgatactcccgaagaggatggaattacctggaccctcgatcagtcctccgaggtcttgggatccggcaaaactctgaccatccaagtcaaggaattcggcgacgcggggcagtacacctgtcacaagggcggagaagtgctgtcgcactcactcctgctccttcacaaaaaggaggacggcatctggtcgaccgacatcctgaaggaccagaaggaacccaagaacaagacctttctgcgctgcgaggccaagaactattcgggaaggttcacctgttggtggctgactaccatctccaccgacctgactttctccgtgaagtcctctcggggttcgagcgacccgcagggtgttacgtgcggtgctgcaaccctgtccgcggagagagtgcggggggacaacaaggaatacgagtactcagtggaatgccaggaagatagcgcctgccctgccgccgaagagtccctgccgattgaagtcatggtggacgcagtgcataagttgaaatatgagaactacacctcgtcgttcttcatccgggacatcatcaagcctgacccccctaagaatctgcagctcaagcccctcaagaactccagacaggtcgaagtgtcctgggagtacccagatacgtggagcacaccgcactcgtacttctccttgaccttctgcgtccaagtgcagggaaagtccaaacgggagaagaaggaccgcgtgttcactgataagacttccgctactgtgatctgccgcaaaaacgccagcatcagcgtgcgcgcgcaagatagatactactcaagctcttggtccgaatgggcgtccgtgccatgctcggtgcccggcgtgggcgtgcctggagtgggagcccggaacttgccggtggccacccctgaccccggaatgttcccttgcctgcaccactcccaaaaccttctgagggctgtgtccaacatgctgcagaaggctcggcagaccctggaattctacccctgcacctccgaggagatcgaccacgaagatattaccaaggacaagacctcaaccgtggaagcctgcctgcccctggaactgaccaagaacgaatcgtgcctgaatagccgggaaacctccttcatcaccaacggctcctgcctggcctcacgaaagaccagctttatgatggccctgtgcctgagctcgatctacgaggacctgaagatgtaccaggtcgagttcaagactatgaacgccaagctgctgatggatccgaagcggcagatcttcttggaccagaatatgctggcagtgatcgacgagctgatgcaggccctcaacttcaactccgagactgtgccgcaaaagtcgagcctggaggaaccggacttctacaagaccaagatcaagttatgtattctcctgcacgcgtttaggattcgcgccgtgaccattgatagagtgatgtcctacctgaacgccagctga

SEQ ID NO: 6 - protein, 536 aa, Artificial Sequence
  Description of Artificial Sequence Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - DNA, 4156 bp, Artificial Sequence
  Description of Artificial Sequence Synthetic polynucleotide
ccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgacgccgccaccatgtgccatcagcaacttgtcatctcctggttctccctcgtgttcctggcctcccctcttgtcgcgatttgggagctgaagaaagatgtgtacgtcgtggaactcgactggtacccggacgcccccggggaaatggtggtgctcacttgtgatactcccgaagaggatggaattacctggaccctcgatcagtcctccgaggtcttgggatccggcaaaactctgaccatccaagtcaaggaattcggcgacgcggggcagtacacctgtcacaagggcggagaagtgctgtcgcactcactcctgctccttcacaaaaaggaggacggcatctggtcgaccgacatcctgaaggaccagaaggaacccaagaacaagacctttctgcgctgcgaggccaagaactattcgggaaggttcacctgttggtggctgactaccatctccaccgacctgactttctccgtgaagtcctctcggggttcgagcgacccgcagggtgttacgtgcggtgctgcaaccctgtccgcggagagagtgcggggggacaacaaggaatacgagtactcagtggaatgccaggaagatagcgcctgccctgccgccgaagagtccctgccgattgaagtcatggtggacgcagtgcataagttgaaatatgagaactacacctcgtcgttcttcatccgggacatcatcaagcctgacccccctaagaatctgcagctcaagcccctcaagaactccagacaggtcgaagtgtcctgggagtacccagatacgtggagcacaccgcactcgtacttctccttgaccttctgcgtccaagtgcagggaaagtccaaacgggagaagaaggaccgcgtgttcactgataagacttccgctactgtgatctgccgcaaaaacgccagcatcagcgtgcgcgcgcaagatagatactactcaagctcttggtccgaatgggcgtccgtgccatgctcggtgcccggcgtgggcgtgcctggagtgggagcccggaacttgccggtggccacccctgaccccggaatgttcccttgcctgcaccactcccaaaaccttctgagggctgtgtccaacatgctgcagaaggctcggcagaccctggaattctacccctgcacctccgaggagatcgaccacgaagatattaccaaggacaagacctcaaccgtggaagcctgcctgcccctggaactgaccaagaacgaatcgtgcctgaatagccgggaaacctccttcatcaccaacggctcctgcctggcctcacgaaagaccagctttatgatggccctgtgcctgagctcgatctacgaggacctgaagatgtaccaggtcgagttcaagactatgaacgccaagctgctgatggatccgaagcggcagatcttcttggaccagaatatgctggcagtgatcgacgagctgatgcaggccctcaacttcaactccgagactgtgccgcaaaagtcgagcctggaggaaccggacttctacaagaccaagatcaagttatgtattctcctgcacgcgtttaggattcgcgccgtgaccattgatagagtgatgtcctacctgaacgccagctgagaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggacggccgctagcaccgttggtttccgtagtgtagtggttatcacgttcgcctaacacgcgaaaggtccccggttcgaaaccgggcactacaaaccaacaacgttaaaaaacaggtcctccccatactctttcattgtacacaccgcaagctcgacaatcatcggattgaagcattgtcgcacacatcttccacacaggatcagtacctgctttcgcttttaaccaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccagcccgcctaatgagcgggcttttttttcttagggtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttcccctaccggggccgctagc

SEQ ID NO: 8 - protein, 10 aa, Artificial Sequence
  Description of Artificial Sequence Synthetic peptide
VPGVGVPGVG

SEQ ID NO: 9 - protein, 5 aa, Artificial Sequence
  Description of Artificial Sequence Synthetic peptide
GGGGS

SEQ ID NO: 10 - protein, 20 aa, Artificial Sequence
  Description of Artificial Sequence Synthetic peptide
GSGSSRGGSGSGGSGGGGSK

(19) United States
(12) Patent Application Publication
(10) Pub. No.: US 2022/0370637 A1
(43) Pub. Date: Nov. 24, 2022

(54) CHITOSAN POLYPLEX-BASED LOCALIZED EXPRESSION OF IL-12 ALONE OR IN COMBINATION WITH TYPE-I IFN INDUCERS FOR TREATMENT OF MUCOSAL CANCERS

(71) Applicant: ENGENE, INC, Montréal (CA)

(72) Inventors: Rajesh Krishnan Gopalakrishna PANICKER, Montreal (CA); Daniel VEILLEUX, Montreal (CA); Pei Lian MA, Montreal (CA); Natalie Chin Mun TAM, Montreal (CA); Carlos FLEET, Montreal (CA); Anthony CHEUNG, Montreal (CA); Shauna DAUPHINEE, Montreal (CA); Ximin CHEN, Montreal (CA); Jose LORA, Montreal (CA)

(21) Appl. No.: 17/438,922

(22) PCT Filed: Mar. 13, 2020
(86) PCT No.: PCT/IB2020/000178

§ 371 Date: Sep. 13, 2021

Related U.S. Application Data
(60) Provisional application No. 62/818,425, filed on Mar. 14, 2019.
(60) Provisional application No. 62/923,403, filed on Oct. 18, 2019.
(60) Provisional application No. 62/924,131, filed on Oct. 21, 2019.

(51) Int. Cl. A61K 47/69; A61K 31/7088; A61K 38/20; A61K 47/60

(52) U.S. Cl. CPC A61K 47/6939; A61K 31/7088; A61K 38/208; A61K 47/60

(57) ABSTRACT

The present disclosure relates to methods and compositions comprising derivatized-chitosan polyplexes reversibly coated with a polyanion-containing block co-polymer for the localized expression of IL-12 in mucosal tissues, preferably in combination with an IFN-1 activator/inducer, for use in cancer immunotherapy.

### CROSS-REFERENCES TO RELATED APPLICATIONS

This application claims the benefit of priority to U.S. Provisional Application Nos. 62/818,425, filed Mar. 14, 2019; 62/923,403, filed Oct. 18, 2019; and 62/924,131, filed Oct. 21, 2019, the contents of which are hereby incorporated by reference in their entirety and for all purposes.

### FIELD OF THE INVENTION

The present disclosure relates to methods and compositions for the localized expression of IL-12 in mucosal tissues, preferably in combination with a type I IFN (IFN-1) activator/inducer, for use in cancer immunotherapy.

### BACKGROUND OF THE INVENTION

Th1 T cells are classically defined by their production of the cytokines IFN-γ, GM-CSF, IL-2, and lymphotoxin (LT, TNF-β). The critical decision to produce Th1 cells is dependent upon the innate immune response to infection by intracellular bacteria, fungi, and viruses. Infection with these pathogens leads to the activation of TLR signaling and the subsequent production of cytokines critical for Th1 development by dendritic cells and NK cells. Th1 polarization and differentiation is promoted by IL-12, IL-18, IFN-γ, and type 1 interferons while being inhibited by IL-4, IL-10, and TGF-β.

IL-12 produced by mature dendritic cells is considered the critical factor associated with Th1 commitment. T-cell activation and STAT1 signals lead to the expression of IL-12Rβ, which upon binding of IL-12 signals through Jak-2 and Tyk-2 to activate STAT 4. STAT4 enters the nucleus where it elicits the expression of Th1-lineage-specific transcription factors such as Tbet. Tbet serves to reinforce the Th1 phenotype by promoting IFN-γ and IL-12Rβ2 expression. IFN-γ, in turn, enhances Th1 differentiation through STAT1 signaling downstream of the IFN-γ receptor, stimulating the production of Tbet. A variety of cells such as NK cells and previously committed Th1 cells are important early sources of IFN-γ. IL-18, in contrast, plays a dual role in Th1 function by promoting Th1 commitment and eliciting IFN-γ production by fully differentiated Th1 cells.

Given its integral role in both innate and adaptive immune response, by activation of NK cells and differentiation of naïve CD4+ cells into Th1 cells, and acting as a potent stimulator of IFN-γ, IL-12 has long been considered a potential candidate for tumor immunotherapy. IL-12 was shown to exhibit anti-tumor activity in vitro (Kobayashi et al., J. Exp. Med. 170:827-845 (1989) and Stern et al., PNAS 87:6808-6812 (1990), and early animal studies appeared to be promising, showing that IL-12 induced tumor regression and reduction in murine tumor models. Studies of systemic IL-12 administration include Brunda et al., (J. Exp. Med., 178:1223-1230 (1993)), which showed that intraperitoneal administration of IL-12 in mice showed markedly reduced experimental pulmonary metastases or subcutaneous growth of B16F10 melanoma. The response to IL-12 administration was dose-dependent, and yielded increased survival times. Similar activity was demonstrated with experimental hepatic metastases and established subcutaneous M5076 reticulum cell sarcoma and Renca renal cell adenocarcinoma tumors. Similarly, Teicher et al., (Int. J. Cancer, 65(1):80-84 (1996), reported that IL-12 was an active anti-tumor agent in three types of solid murine tissues: B16 melanoma, Lewis lung carcinoma, and renal cell carcinoma, while Kozar et al, (Clin. Can. Res., 9(8):3124-3133 (2003) showed that mice inoculated with L1210 leukemia cells or with B16F10 melanoma cells treated with daily injections or doses of IL-12 showed moderate reduction of tumors.

In these animal studies little or no apparent toxicity was reported, making IL-12 an attractive molecule for further clinical development. Unfortunately, however, decreased efficacy and increased toxicity was seen in early human clinical trials. In the first phase 1 dose escalation study, I.V. administration of IL-12 to forty patients with advanced solid tumors (including melanomas, renal and colon cancer) yielded only one complete response in a patient with melanoma and a partial response in a patient with renal cancer (Atkins et al., Clin. Cancer Res., 3:409-17 (1997)). Significant systemic toxicity was seen in this study, with three of the four patients treated with the highest dosage of IL-12 (1000 ng/kg) experiencing stomatitis and/or hepatic dysfunction. Bajetta et al., (Clin. Cancer Res., 4:75-85 (1998), in a pilot study of subcutaneous injections of 0.5 ug/kg IL-12 in ten patients with metastatic melanoma, showed flu-like syndrome in 100% of patients, fever in 90% of patients, transitory hypertransaminasemia in 60% of patients and hypertriglyceridemia in 80% of patients. Additional types of toxicity were seen in 10-20% of the study patients. No partial or complete responses were observed, although tumor regression was seen in three of the ten participants. Most notably, in a phase II trial performed by Genetics Institute, administering IL-12 intravenously on consecutive days resulted in hospitalization of 12 of the 17 patients enrolled, and two patient deaths (Leonard et al., Blood, 90(7):2541-2548 (1995)).

To avoid the toxicity seen with systemic administration, studies were done using localized delivery of IL-12. While these studies showed less toxicity associated with administration of IL-12 than the systemic studies, localized delivery was largely ineffective in eliminating tumors. Lenzi et al. (Clin. Cancer Res., 8:3686-3695, (2002)), administered IL-12 via a peritoneal catheter to 29 patients suffering from peritoneal carcinomatosis attributable to ovarian and various abdominal cancers in a Phase 1 trial, at doses ranging from 3 ng/kg up to 600 ng/kg. Although no life-threatening toxicities were seen in the trial, only 2 of the 29 patients enrolled experienced complete response, 8 had stable disease and the remaining 19 had progressive disease. Similarly, Weiss et al. (J. Immunother., 26(4):343-348 (2003) showed intravesical administration of IL-12 to patients with superficial transitional cell carcinoma of the bladder resulted in no moderate, severe, or life-threatening toxicity, but the patients also showed no clinically relevant evidence of antitumor or immunologic effects.

The use of chitosan as an adjuvant for IL-12 for the treatment of cancer has been reported. Chitosan was used as part of a coformulation with IL-12 for intravesical immunotherapy for the treatment of bladder cancer (Zaharoff et al., Cancer Res. 69(15):6192-6199 (2009), and intratumoral injection for colorectal and pancreatic tumors (Zaharoff et al, J. immunotherapy, 33(7):697-705 (2010) and breast cancer (Vo et al., Oncoimmunology, 3912):e968001 (2015) in mice. To reduce dissemination of IL-12 from the site of treatment, and avoid possible toxicity due to IL-12 dissemination in the system, the use of IL-12 molecule covalently linked to chitosan as a therapeutic agent has also been contemplated (Zaharoff et al., U.S. 20170106092). Unfortunately, however, the clinical application of IL-12-based therapies remains problematic due to the potential for rapid development of lethal inflammatory syndrome, and improved strategies to overcome IL-12-mediated toxicity are still needed. Wang et al., Nature Communications 8, Article 1395 (2017).

Thus, there remains a need for new compositions and methods for the effective localized expression of IL-12, but with reduced toxicity and improved efficacy. Ideally, these could be used in conjunction with additional immune stimulation strategies to enhance cancer immunotherapy in the tumor microenvironment. The current knowledge base regarding IL-12 combination therapies is quite limited, however, and has focused primarily on the co-administration and/or co-expression of IL-12 with an additional immunomodulatory cytokine such as IL-2, IL-7, IL-15, IL-18 and IL-21. See, e.g. Weiss et al., Expert Opin Biol Ther. 7:1705-1721 (2007). Notably, the potential synergistic interaction between IL-12 and alternative innate and/or adaptive immune stimulation strategies has been largely unexplored.

Pattern recognition receptors (PRR) comprise another element of the innate immune system, recognizing conserved pathogen-associated nucleic acid (NA) sequences. PRR activation by their nucleotide ligand induces production of type I interferons (IFN) and proinflammatory cytokines, serving as the first line of defense for viral and microbial infections. Iwasaki and Medzhitov, Science 327:291-95 (2010). These NA-sensing PRRs include the endosomal toll-like receptor (TLR) family (Majer et al., Curr. Opin. Immunol. 44:26-33 (2017)), the cytosolic DNA sensors AIM 2 and cyclic GMP-AMP synthetase (cGAS), part of the cGAS-stimulator of interferon (STING) pathway (Chen et al., Nat. Immunol. 17:1142-49 (2016)), and the cytosolic RNA sensors retinoic acid-inducible gene I (RIG-I)-like receptor family (Schlee, Immunobiology, 218:1322-35 (2013)). Interestingly, there are remarkably few references evaluating both IL-12 and RIG-I, and the few that do demonstrate no meaningful interaction between the two. Jiang et al. (J. Exp. Med. 216:2854-68 (2019)) showed that eliminating endogenous IL-12 in vivo by way of a neutralizing antibody had no effect on an induced RIG-I response, while the earlier studies by Kong et al. (Cell Host & Microbe 6:150-61 (2009)) showed that altering levels of RIG-I had no effect upon IL-12 expression in cell-based assays. As such, the combination of IL-12 and RIG-I in particular would appear to be unavailing.

### SUMMARY OF THE INVENTION

The present invention resolves the still unmet need in the art for the effective localized expression of IL-12, employing derivatized-chitosan polyplexes reversibly coated with a polyanion-containing block co-polymer for robust transfection of mucosal tissues present in or proximal to a tumor. Moreover, as presented herein for the first time, the effective localized expression of IL-12 obtained with the subject compositions and methods can be advantageously combined with one or more additional innate and/or adaptive immune stimulation strategies to further enhance the cytotoxic immune response against the tumor. In preferred embodiments, IL-12 expression according to the subject invention is combined with the simultaneous or sequential administration and/or expression of a Type I interferon (IFN-1) activator/inducer, e.g. a RIG-I agonist, a STING agonist, and/or a TLR 7/9 agonist. In some embodiments, the subject methods and compositions comprise the co-expression of IL-12 with at least one RIG-I agonist.

In one aspect, the invention provides a composition comprising a derivatized-chitosan nucleic acid polyplex comprising amino-functionalized chitosan and a therapeutic nucleic acid construct encoding IL-12, wherein said derivatized-chitosan nucleic acid polyplex further comprises a reversible coating comprising one or more polyanion-containing block co-polymers having at least one polyanionic anchor region and at least one hydrophilic tail region.

In preferred embodiments, the polyanion-containing block co-polymer is a linear diblock and/or triblock co-polymer. In preferred embodiments, the amino-functionalized chitosan further comprises a hydrophilic polyol. In some embodiments, the amino-functionalized chitosan comprises arginine. In some embodiments, the hydrophilic polyol is glucose or gluconic acid.

In some embodiments, the therapeutic nucleic acid construct is comprised within a plasmid selected from the group consisting of: gWIZ, pVAX, NTC8685 or NTC9385R. In some embodiments, the therapeutic nucleic acid construct further comprises an expression control element selected from the group consisting of CMV, EF1a, CMV/EF1a, and CAG, and CMV/EF1α/HTLV. In some embodiments, the therapeutic nucleic acid construct comprises a synthetic beta-globin-based intron. In some embodiments, the therapeutic nucleic acid construct comprises a HTLV-IR. In some embodiments, the therapeutic nucleic acid construct comprises a kanamycin or sucrose-based selection element. In some embodiments, the therapeutic nucleic acid construct comprises a pUC or R6K origin of replication.

In some embodiments, the subject therapeutic nucleic acid construct further comprises a nucleic acid encoding at least one additional innate and/or adaptive immunostimulatory molecule. In some embodiments, the immunostimulatory molecule comprises a Type I interferon (IFN-1) activator/inducer, e.g. a RIG-I agonist, a STING agonist, and/or a TLR 7/9 agonist. In preferred embodiments the therapeutic nucleic acid construct further comprises a nucleic acid encoding at least one RIG-I agonist, e.g., eRNA11a, adenovirus VA RNA1, eRNA41H, MK4621, SLR10, SLR14, and/or SLR20, and still more preferably, eRNA11a or eRNA41H. In some embodiments the immunostimulatory molecule is a modulator of an immune checkpoint inhibitor.

In another aspect, methods are provided for the localized expression of IL-12 in a mucosal tissue in a patient in need thereof, comprising administering to said patient a therapeutically-effective amount of a pharmaceutical composition comprising a derivatized-chitosan nucleic acid polyplex comprising amino-functionalized chitosan and a therapeutic nucleic acid construct encoding IL-12, wherein said derivatized-chitosan nucleic acid polyplex further comprises a reversible coating comprising one or more polyanion-containing block co-polymers having at least one polyanionic anchor region and at least one hydrophilic tail region.

In some embodiments, the methods further comprise co-expression of an IFN-1 activator/inducer and/or an immune checkpoint inhibitor. In some embodiments, the IFN-1 activator/inducer and/or immune checkpoint inhibitor is co-expressed with IL-12 from the same or a different therapeutic nucleic acid construct. In preferred embodiments, the therapeutic nucleic acid construct comprises a nucleic acid encoding a single-chain hIL-12 and at least one RIG-I agonist, e.g., eRNA11a, VA RNA1, eRNA41H, MK4621, SLR10, SLR14, and SLR20, and still more preferably, eRNA11a or eRNA41H. In exemplary embodiments, the nucleic acid encoding single-chain hIL-12 comprises SEQ ID NO: 7.

In some embodiments, the IFN-1 activator/inducer and/or immune checkpoint inhibitor is separately administered. In preferred embodiments, the immune checkpoint inhibitor is selected from the group consisting of ipilimumab, tremelimumab, nivolumab, atezolizumab and/or pembrolizumabco. In some embodiments, the IFN-1 activator/inducer is MK 4621/JetPEI.

In another aspect, methods are provided for treating a mucosal cancer in a patient in need thereof, comprising administering to said patient a therapeutically effective amount of a pharmaceutical composition comprising a derivatized-chitosan nucleic acid polyplex comprising amino-functionalized chitosan and a therapeutic nucleic acid encoding IL-12, wherein said derivatized-chitosan nucleic acid polyplex further comprises a reversible coating comprising one or more polyanion-containing block co-polymers having at least one polyanionic anchor region and at least one hydrophilic tail region.

In some embodiments, the methods further comprise co-expression of an IFN-1 activator/inducer and/or an immune checkpoint inhibitor. In some embodiments, the IFN-1 activator/inducer and/or immune checkpoint inhibitor is co-expressed with IL-12 from the same or a different therapeutic nucleic acid construct. In preferred embodiments, the therapeutic nucleic acid construct comprises a nucleic acid encoding a single-chain hIL-12 and at least one RIG-I agonist, e.g., eRNA11a, VA RNA1, eRNA41H, MK4621, SLR10, SLR14, and SLR20, and still more preferably, eRNA11a or eRNA41H. In exemplary embodiments, the nucleic acid encoding single-chain hIL-12 comprises SEQ ID NO: 7.

In some embodiments, the IFN-1 activator/inducer and/or immune checkpoint inhibitor is separately administered. In preferred embodiments, the immune checkpoint inhibitor is selected from the group consisting of ipilimumab, tremelimumab, nivolumab, atezolizumab and/or pembrolizumabco. In some embodiments, the IFN-1 activator/inducer is MK 4621/JetPEI.

In preferred embodiments, the polyanion-containing block co-polymer is a linear diblock and/or triblock co-polymer. In preferred embodiments, the amino-functionalized chitosan further comprises a hydrophilic polyol. In some embodiments, the amino-functionalized chitosan comprises arginine. In some embodiments, the hydrophilic polyol is glucose or gluconic acid.

In another aspect, the invention provides a pharmaceutical composition comprising a nucleic acid polyplex comprising a cationic polymer and a therapeutic nucleic acid construct encoding IL-12 and at least one RIG-I agonist. In preferred embodiments, the therapeutic nucleic acid construct encodes a single chain hIL-12 molecule and at least one RIG-I agonist selected from the group consisting of eRNA11a, VA RNA1, eRNA41H, MK4621, SLR10, SLR14, and SLR20, and still more preferably from the group consisting of eRNA11a or eRNA41H. In exemplary embodiments, the nucleic acid encoding single-chain hIL-12 comprises SEQ ID NO: 7.

In some embodiments, the cationic polymer is selected from the group comprising polyamines; polyorganic amines, poly(amidoamines); polyamino acids polyethyleneimine celluloses, polysaccharides, chitosan, and derivatives thereof. In some embodiments, the cationic polymer is selected from the group consisting of polyethyleneimine (PEI), PAMAM, polylysine (PLL), polyarginine, chitosan, and derivatives thereof.

In preferred embodiments, the cationic polymer comprises a derivatized chitosan. In the particularly preferred embodiments exemplified herein, the derivatized chitosan is an amino-functionalized chitosan, and more preferably a dually-derivatized chitosan comprising arginine and a hydrophilic polyol, e.g. gluconic acid or glucose. In some embodiments, the nucleic acid polyplex further comprises a reversible coating comprising one or more polyanion-containing block co-polymers having at least one polyanionic anchor region and at least one hydrophilic tail region. In preferred embodiments, the polyanion-containing block co-polymer is a linear diblock and/or triblock co-polymer.

In some embodiments, the therapeutic nucleic acid construct is comprised within a plasmid selected from the group consisting of: gWIZ, pVAX, NTC8685 or NTC9385R. In some embodiments, the therapeutic nucleic acid construct further comprises an expression control element selected from the group consisting of CMV, EF1a, CMV/EF1a, and CAG, and CMV/EF1α/HTLV. In some embodiments, the therapeutic nucleic acid construct comprises a synthetic beta-globin-based intron. In some embodiments, the therapeutic nucleic acid construct comprises a HTLV-IR. In some embodiments, the therapeutic nucleic acid construct comprises a kanamycin or sucrose-based selection element. In some embodiments, the therapeutic nucleic acid construct comprises a pUC or R6K origin of replication.

In another aspect, methods are provided for the localized expression of IL-12 in a mucosal tissue in a patient in need thereof, comprising administering to said patient a therapeutically-effective amount of a pharmaceutical composition comprising a nucleic acid polyplex comprising a cationic polymer and a therapeutic nucleic acid construct encoding IL-12 and at least one RIG-I agonist. In preferred embodiments, the therapeutic nucleic acid construct encodes a single chain hIL-12 molecule and at least one RIG-I agonist selected from the group consisting of eRNA11a, VA RNA1, eRNA41H, MK4621, SLR10, SLR14, and SLR20, and still more preferably from the group consisting of eRNA11a or eRNA41H. In exemplary embodiments, the nucleic acid encoding single-chain hIL-12 comprises SEQ ID NO: 7.

In another aspect, methods are provided for treating a mucosal cancer in a patient in need thereof, comprising administering to said patient a therapeutically effective amount of a pharmaceutical composition comprising a nucleic acid polyplex comprising a cationic polymer and a therapeutic nucleic acid encoding IL-12 and at least one RIG-I agonist. In preferred embodiments, the therapeutic nucleic acid construct encodes a single chain hIL-12 molecule and at least one RIG-I agonist selected from the group consisting of eRNA11a, VA RNA1, eRNA41H, MK4621, SLR10, SLR14, and SLR20, and still more preferably from the group consisting of eRNA11a or eRNA41H. In exemplary embodiments, the nucleic acid encoding single-chain hIL-12 comprises SEQ ID NO: 7.

In some embodiments, the cationic polymer is selected from the group comprising polyamines; polyorganic amines, poly(amidoamines); polyamino acids polyethyleneimine celluloses, polysaccharides, chitosan, and derivatives thereof. In some embodiments, the cationic polymer is selected from the group consisting of polyethyleneimine (PEI), PAMAM, polylysine (PLL), polyarginine, chitosan, and derivatives thereof.

In preferred embodiments, the cationic polymer comprises a derivatized chitosan. In the particularly preferred embodiments exemplified herein, the derivatized chitosan is an amino-functionalized chitosan, and more preferably a dually-derivatized chitosan comprising arginine and a hydrophilic polyol, e.g. gluconic acid or glucose. In some embodiments, the nucleic acid polyplex further comprises a reversible coating comprising one or more polyanion-containing block co-polymers having at least one polyanionic anchor region and at least one hydrophilic tail region. In preferred embodiments, the polyanion-containing block co-polymer is a linear diblock and/or triblock co-polymer.

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, where indicated, the term “about” indicates the designated value±one standard deviation of that value.

The term “combinations thereof” includes every possible combination of elements to which the term refers.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder. For example, in an exemplary embodiment, the phrase “treating cancer” refers to inhibition of cancer cell proliferation, inhibition of cancer spread (metastasis), inhibition of tumor growth, reduction of cancer cell number or tumor growth, decrease in the malignant grade of a cancer (e.g., increased differentiation), or improved cancer-related symptoms.

As used herein, the term “therapeutically effective amount” or “effective amount” refers to an amount of the subject compositions that when administered to a subject is effective to treat a disease or disorder. For example, in an exemplary embodiment, the phrase “effective amount” is used interchangeably with “therapeutically effective amount” or “therapeutically effective dose” and the like, and means an amount of a therapeutic agent that is effective for treating cancer. Effective amounts of the compositions provided herein may vary according to factors such as the disease state, age, sex, weight of the animal.

As used herein, the term “subject” or “individual” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has cancer, an autoimmune disease or condition, and/or an infection that can be treated with an antibody provided herein. In some embodiments, the subject is a human that is suspected to have cancer, an autoimmune disease or condition, and/or an infection.

“Chitosan” is a partially or entirely deacetylated form of chitin, a polymer of N-acetylglucosamine. Chitosans with any degree of deacetylation greater than 50% are used in the present invention.

Chitosan may be derivatized by functionalizing free amino groups at the sites of deacetylation. The derivatized chitosans described herein have a number of properties which are advantageous for a nucleic acid delivery vehicle including: they effectively bind and complex the negatively charged nucleic acids, they can be formed into nanoparticles of a controllable size, they can be taken up by the cells and they can release the nucleic acids at the appropriate time within the cells. Chitosans with any degree of functionalization between 1% and 50%. (Percent functionalization is determined relative to the number of free amino moieties on the chitosan polymer prior-to or in the absence of functionalization.) The degrees of deacetylation and functionalization impart a specific charge density to the functionalized chitosan derivative.

A polyol according to the present invention may have a 3, 4, 5, 6, or 7 carbon backbone and may have at least 2 hydroxyl groups. Such polyols, or combinations thereof, may be useful for conjugation to a chitosan backbone, such as a chitosan that has been functionalized with a cationic moiety (e.g., a molecule comprising an amino group such as, lysine, ornithine, a molecule comprising a guanidinium group, arginine, or a combination thereof).

The term “C₂-C₆ alkylene” as used herein refers to a linear or branched divalent hydrocarbon radical optionally containing one or more carbon-carbon multiple bonds. For the avoidance of doubt, the term “C₂-C₆ alkylene” as used herein encompasses divalent radicals of alkanes, alkenes and alkynes.

As used herein, unless otherwise indicated, the term “peptide” and “polypeptide” are used interchangeably.

The term “polypeptide” is used in its broadest sense to refer to conventional polypeptides (i.e., short polypeptides containing L or D-amino acids), as well as peptide equivalents, peptide analogs and peptidomimetics that retain the desired functional activity. Peptide equivalents can differ from conventional peptides by the replacement of one or more amino acids with related organic acids, amino acids or the like, or the substitution or modification of side chains or functional groups.

Peptidomimetics may have one or more peptide linkages replaced by an alternative linkage, as is known in the art. Portions or all of the peptide backbone can also be replaced by conformationally constrained cyclic alkyl or aryl substituents to restrict mobility of the functional amino acid sidechains, as is known in the art.

The polypeptides of this invention may be produced by recognized methods, such as recombinant and synthetic methods that are well known in the art. Techniques for the synthesis of peptides are well known and include those described in Merrifield, J. Amer. Chem. Soc. 85:2149-2456 (1963), Atherton, et al., Solid Phase Peptide Synthesis: A Practical Approach, IRL Press (1989), and Merrifield, Science 232:341-347 (1986).

As used herein, “linear polypeptide” refers to a polypeptide that lacks branching groups covalently attached to its constituent amino acid side chains. As used herein, “branched polypeptide” refers to a polypeptide that comprises branching groups covalently attached to its constituent amino acid side chains.

The “final functionalization degree” of cation or polyol as used herein refers to the percentage of cation (e.g., amino) groups on the chitosan backbone functionalized with cation (e.g., amino) or polyol, respectively. Accordingly, “V ratio”, “final functionalization degree ratio” (e.g., Arginine final functionalization degree:polyol final functionalization degree ratio) and the like may be used interchangeably with the term “molar ratio” or “number ratio.”

Dispersed systems consist of particulate matter, known as the dispersed phase, distributed throughout a continuous medium. A “dispersion” of chitosan nucleic acid polyplexes is a composition comprising hydrated chitosan nucleic acid polyplexes, wherein polyplexes are distributed throughout the medium.

As used herein, a “pre-concentrated” dispersion is one that has not undergone the concentrating process to form a concentrated dispersion.

As used herein, “substantially free” of polyplex precipitate means that the composition is essentially free from particles that can be observed on visual inspection.

As used herein, physiological pH refers to a pH between 6 to 8.

By “chitosan nucleic acid polyplex” or its grammatical equivalents is meant a complex comprising a plurality of chitosan molecules and a plurality of nucleic acid molecules. In a preferred embodiment, the (e.g., dually-) derivatized-chitosan is complexed with said nucleic acid.

The term “polyethylene glycol” (“PEG”) as used herein is intended to mean a polymer of ethylene oxide having repeat units of —(CH2CH2—O)— and the general formula of HO—(CH2CH2—O)n—H.

The term “monomethoxy polyethylene glycol” (“mPEG”) as used herein is intended to mean a polymer of ethylene oxide having repeat units of —(CH2CH2—O)— and the general formula of CH3O—(CH2CH2—O)n—H, for example, a PEG capped at one end with a methoxy group.

### BRIEF DESCRIPTION OF THE DRAWINGS

The present disclosure is disclosed with reference to the accompanying drawings, wherein:

FIG. 1 shows dose-dependent enzymatic activity of a luciferase reporter gene (Luc2) following in vitro transfection of plasmids of interest into MB49 cells.

FIG. 2A illustrates the rank-ordering of vector backbones of interest based on transfection efficiency in vivo (mRNA copy number of reporter gene).

FIG. 2B illustrates the rank-ordering of vector backbones of interest based on transfection efficiency in vivo (enzymatic activity of reporter gene).

FIG. 2C illustrates the rank-ordering of vector backbones of interest in vivo based on mRNA copy number in transfected tissue.

FIG. 2D illustrates the rank-ordering of vector backbones of interest based on levels of protein expressed in vivo.

FIG. 3A shows GFP expression levels in vitro using MB49 cells transfected with plasmids comprising promoter/enhancer sequences of interest.

FIG. 3B illustrates the rank-ordering of promoter/enhancer sequences based on in vitro expression of GFP in MB49 cells.

FIG. 4A shows in vitro GFP fluorescence results for human primary bladder epithelial cells transfected with plasmids containing promoter/enhancer sequences of interest.

FIG. 4B illustrates the rank-ordering of promoter-enhancer sequences based on activity in human primary bladder epithelial cells in vitro.

FIG. 5A illustrates dose-response of GFP expression following in vitro transfection of human primary bladder epithelial cells with plasmids of interest that were retrofitted to remove bacterial components.

FIG. 5B illustrates rank-ordering of retrofit plasmids based on activity in human primary bladder epithelial cells in vitro.

FIG. 6 illustrates the effect of PEGylation on bladder transfection in vivo.

FIGS. 7A and 7B illustrates mRNA expression of human PD-L1-Fc in vivo +/− PEGylation at two different concentrations.

FIG. 8 illustrates levels of protein expression mediated by transfection with PEG-DDX in vivo.

FIG. 9A compares the effect of PEGylation on polyplex stability; appearance of PEGylated and non-PEGylated polyplexes voided after 1 hour of incubation in the bladder.

FIG. 9B shows dynamic light scattering assay results for PEGylated and non-PEGylated polyplexes post-voiding after 1 hour of incubation in the bladder.

FIG. 10 shows effects of DDX-1 (DDX) and DDX-II (RXG) on hPD-L1-Fc production in vivo.

FIG. 11 shows levels of human PD-L1-Fc protein expression following administration of RXG (DDX-II, PEGylated and non-PEGylated) formulations to the mouse bladder.

FIGS. 12A and 12B illustrates the plasmid DNA constructs with mouse IL-12 without and with the RIG-I agonist cassette. FIG. 12A further shows the mIL-12 transgene comprising the p40 and p35 genes in a single open reading frame joined by a short elastin linker. FIG. 12B depicts the schematic representation of a clinical embodiment of a pharmaceutical composition according to the present invention.

FIG. 13A depicts IFNγ production from splenocytes stimulated with plasmid-produced mIL-12.

FIG. 13B depicts the functional activity of human IL-12p40p35 expressed from NTC9385R plasmid backbones.

FIG. 13C shows mouse-mediated SEAP production from an IL-12-responsive reporter cell line (HEKBlue).

FIGS. 14A and 14B compares IFNβ levels from cultured bladder cancer epithelial cells post-transfection with empty plasmids (FIG. 14A) vs. plasmids bearing IL-12 genes with and without the RIG-I agonist cassette (FIG. 14B).

FIG. 15 shows levels of IL-12 mRNA at 4, 24, 48, 72, and 96 hours-post transfection in vivo.

FIGS. 16A and 16B illustrate the Dually-Derivatized Chitosan RXG (DDX II) structure and process for synthesizing the DDX/DNA nanoparticles of the invention. FIG. 16C provides the chemical structure of PEG-b-PLE.

FIG. 17 shows data regarding the physiochemical characterization of DDX-DNA formulations.

FIGS. 18A, 18B, and 18C provide in vitro polymer screening assay results.

FIG. 19 shows in vivo protein expression following intravesical administration in mice comparing DDX vs. RXG (DDX-II)-based polyplexes.

FIGS. 20A and 20B compare IL-12 mRNA (FIG. 20A) and protein (FIG. 20B) in non-human primates after intravesical administration of a polyplex comprising human IL-12.

FIG. 21 shows eRNA11a expression in NHP bladders exposed to polyplex comprising hIL-12 and RIG-I agonist (EG-70) at 0.0625 mg/mL (c62.5), 0.25 mg/mL (c250) and 1 mg/mL (c1000); and non-coding control (RXG-PEG-N9) at 1 mg/mL (c1000).

FIG. 22 shows VA1 expression in NH bladders exposed to polyplex comprising hIL-12 and RIG-I agonist (EG-70) at 0.0625 mg/mL (c62.5), 0.25 mg/mL (c250) and 1 mg/mL (c1000); and non-coding control (RXG-PEG-N9) at 1 mg/mL (c1000).

FIG. 23 illustrates the kinetics of codon-optimized mRNA expression of Il12p40p35 in the mouse bladder following a single administration of mEG-70-prototype nanoparticles.

FIG. 24 shows mouse IL12p70 protein Expression in the mouse bladder following a single administration of mEG-70 prototype nanoparticles.

FIG. 25 shows dose response of mouse IL-12p70 protein expression in the mouse bladder following a single administration of mEG-70 prototype nanoparticles.

FIG. 26 compares PEGylated nanoparticles expression of mouse IL-12p70 protein vs. that of unPEGylated nanoparticles.

FIG. 27A provides the assay protocol for assessment of efficacy of mEG-70 (mIL-12 and RIG-I agonist) for the treatment of bladder cancer in a mouse model. FIG. 27B shows the bladder weight of control and treated animals at the study endpoint.

### DETAILED DESCRIPTION

The present invention contemplates localized expression of IL-12 in mucosal tissues, preferably combined with additional innate and/or adaptive immune stimulation. Parenteral and intravenous routes of protein therapies suffer from the lack of sufficient bioavailability at mucosal tissues and systemic toxicity. Localized gene therapies at mucosal tissue, such as intravesical administration to the bladder and oral dosage form to gastrointestinal tract (GIT), present an attractive approach to promote local expression of proteins while minimizing unwanted systemic side effects. Unfortunately, however, clinical use of viral delivery vectors is limited by viral immunogenicity which could diminish efficacy after repeated dosing; inefficient penetration of mucous barrier; cost of vector production; and logistical complications associated with clinical implementation (e.g. biosafety containment and cold-chain storage). The present invention provides a safe and efficient non-viral vector platform for mucosal tissues, e.g., the bladder, to overcome these limitations.

Without being bound by theory, activation of the IL-12 pathway in mucosal tissues by way of the subject invention acts on effector CD4+ and CD8+ effector cells leading to potent anti-tumor as well as anti-angiogenic functions, whereas simultaneous or sequential stimulation of the RIG-I pathway results in induction of type-I interferons and IFN-stimulated genes, leading to improved cross-presentation of tumor antigens to CD8+ cytotoxic T cells In the preferred embodiments described and exemplified herein, these concerted biological mechanisms are combined to produce a potent inflammatory response driving robust and durable anti-tumor immune responses, coupling stimulation of innate immune system by the RIG-I agonists to the IL12-mediated stimulation of the adaptive immune response.

### Compositions

Provided herein are chitosan compositions comprising a chitosan-derivative nucleic acid nanoparticle (polyplex) in complex with a polyanion-containing block co-polymer, e.g. a diblock and/or triblock co-polymer coating, wherein individual polymer molecules comprise a negatively charged anchor region and one or more non-charged hydrophilic tail regions. Exemplary polymer molecules useful in the methods and compositions of the present invention are “PEG-PA” polymer molecules comprising a polyethylene glycol (PEG) portion and a polyanion (PA) portion.

1.1. Chitosan

The chitosan component of the chitosan-derivative nucleic acid nanoparticle can be functionalized with a cationic functional group and/or a hydrophilic moiety. Chitosan functionalized with two different functional groups is referred to as dually derivatized chitosan (DD-chitosan). Exemplary DD-chitosans are functionalized with both a hydrophilic moiety (e.g., a polyol) and a cationic functional group (e.g., an amino group). Exemplary chitosan derivatives are also described in, e.g., U.S. 2007/0281904; and U.S. 2016/0235863, which are each incorporated herein by reference.

In one embodiment, the dually derivatized chitosan described herein comprises chitosan having a degree of deacetylation of at least 50%. In one embodiment, the degree of deacetylation is at least 60%, more preferably at least 70%, more preferably at least 80%, more preferably at least 90%, and most preferably at least 95%. In a preferred embodiment, the dually derivatized chitosan described herein comprises chitosan having a degree of deacetylation of at least 98%.

The chitosan derivatives described herein have a range of average molecular weights that are soluble at neutral and physiological pH, and include for the purposes of this invention molecular weights ranging from 3-110 kDa. Embodiments described herein feature lower average molecular weight of derivatized chitosans (<25 kDa, e.g., from about 5 kDa to about 25 kDa), which can have desirable delivery and transfection properties, and are small in size and have favorable solubility. A lower average molecular weight derivatized chitosan is generally more soluble than one with a higher molecular weight, the former thus producing a nucleic acid/chitosan complex that will release more easily the nucleic acid and provide increased transfection of cells. Much literature has been devoted to the optimization of all of these parameters for chitosan-based delivery systems.

An ordinarily skilled artisan will recognize that chitosan refers to a plurality of molecules having a structure of Formula I, wherein n is any integer, and each R1 is independently selected from acetyl or hydrogen, wherein the degree of R1 selected from hydrogen is between 50% to 100%. Also, chitosan referred to as having an average molecular weight, e.g., of 3 kD to 110 kD, generally refers to a plurality of chitosan molecules having a weight average molecular weight of, e.g., 3 kD to 110 kD, respectively, wherein each of the chitosan molecules may have different chain lengths (n+2). It is also well recognized that chitosan referred to as “n-mer chitosan,” does not necessarily comprise chitosan molecules of Formula I, wherein each chitosan molecule has a chain length of n+2. Rather, “n-mer chitosan” as used herein refers a plurality of chitosan molecules, each of which may have different chain lengths, wherein the plurality has an average molecule weight substantially similar to or equal to a chitosan molecule having a chain length of n. For example, 24-mer chitosan may comprise a plurality of chitosan molecules, each having different chain lengths ranging from, e.g. 7-50, but which has a weight average molecular weight substantially similar or equivalent to a chitosan molecule having a chain length of 24.

A dually derivatized chitosan of the invention may also be functionalized with a polyol, or a hydrophilic functional group such as a polyol. Without wishing to be bound by theory, it is hypothesized that functionalization with a hydrophilic group such as a polyol which may help to increase the hydrophilicity of chitosan (including Arginine-chitosan) and/or may donate a hydroxyl group. In some embodiments, the hydrophilic functional group of the chitosan-derivative nanoparticles is or comprises gluconic acid. See, e.g., WO 2013/138930. In some embodiments, the hydrophilic functional group of the chitosan-derivative nanoparticles is or comprises glucose. Additionally or alternatively, the hydrophilic functional group can comprise a polyol. See, e.g., U.S. 2016/0235863. Exemplary polyols for functionalization of chitosan are further described below.

The functionalized chitosan derivatives described herein include dually derivatized-chitosan compounds, e.g., cation-chitosan-polyol compounds. In general, the cation-chitosan-polyol compounds are functionalized with an amino-containing moiety, such as an arginine, lysine, ornithine, or molecule comprising a guanidinium, or a combination thereof. In certain embodiments, the cation-chitosan-polyol compounds have the following structure of Formula I.

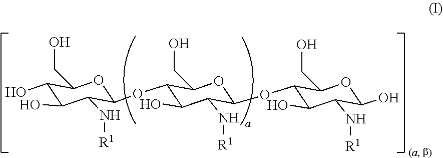

wherein n is an integer of 1 to 650,
α is the final functionalization degree of the cation moiety (e.g., a molecule comprising an amino group such as, lysine, ornithine, a molecule comprising a guanidinium group, arginine, or a combination thereof),
β is the final functionalization degree of polyol; and
each R¹ is independently selected from hydrogen, acetyl, a cation (e.g., arginine), and a polyol.

Preferably, a dually derivatized chitosan of the invention may be functionalized with the cationic amino acid, arginine.

In one embodiment, the chitosan-derivative nanoparticle comprises chitosan coupled with gluconic acid at a final functionalization degree of 1%, 2%, 4%, 7%, 8%, 10%, 15%, 20%, 25%, 30%, or greater. In one embodiment, the chitosan-derivative nanoparticle comprises chitosan coupled with glucose at a final functionalization degree of 1%, 2%, 4%, 7%, 8%, 10%, 15%, 20%, 25%, 30%, or greater. In one embodiment, the chitosan derivative nanoparticle comprises chitosan coupled with a cationic moiety (e.g., arginine) at a final functionalization degree of from about 1% to about 25%. In one embodiment, the chitosan derivative nanoparticle comprises chitosan coupled with a cationic moiety (e.g., arginine) at a final functionalization degree of from about 10% to about 40%.

In one embodiment, the chitosan derivative nanoparticle comprises chitosan coupled with a cationic moiety (e.g., arginine) at a final functionalization degree of from about 10% to about 35%. In one embodiment, the chitosan derivative nanoparticle comprises chitosan coupled with a cationic moiety (e.g., arginine) at a final functionalization degree of from about 20% to about 35%. In one embodiment, the chitosan derivative nanoparticle comprises chitosan coupled with a cationic moiety (e.g., arginine) at a final functionalization degree of from about 25% to about 35%. In one embodiment, the chitosan derivative nanoparticle comprises chitosan coupled with a cationic moiety (e.g., arginine) at a final functionalization degree of from about 25% to about 30%.

In one embodiment, the chitosan derivative nanoparticle comprises chitosan coupled with a cationic moiety (e.g., arginine) at a final functionalization degree of from about 15% to about 40%. In one embodiment, the chitosan derivative nanoparticle comprises chitosan coupled with a cationic moiety (e.g., arginine) at a final functionalization degree of from about 15% to about 35%. In one embodiment, the chitosan derivative nanoparticle comprises chitosan coupled with a cationic moiety (e.g., arginine) at a final functionalization degree of from about 15% to about 30%. In one embodiment, the chitosan derivative nanoparticle comprises chitosan coupled with a cationic moiety (e.g., arginine) at a final functionalization degree of from about 15% to about 28%.

In one embodiment, the chitosan derivative nanoparticle comprises chitosan coupled with a cationic moiety (e.g., arginine) at a final functionalization degree of from about 10% to about 35%. In one embodiment, the chitosan derivative nanoparticle comprises chitosan coupled with a cationic moiety (e.g., arginine) at a final functionalization degree of from about 10% to about 30%. In one embodiment, the chitosan derivative nanoparticle comprises chitosan coupled with a cationic moiety (e.g., arginine) at a final functionalization degree of from about 10% to about 28%. In one embodiment, the chitosan derivative nanoparticle comprises chitosan coupled with a cationic moiety (e.g., arginine) at a final functionalization degree of about 28%.

In one embodiment, the chitosan-derivative nanoparticle comprises chitosan coupled with gluconic acid at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with gluconic acid at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with gluconic acid at a final functionalization degree of about 10%.

In one embodiment, the chitosan-derivative nanoparticle comprises chitosan coupled with hydrophilic polyol at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with hydrophilic polyol at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with hydrophilic polyol at a final functionalization degree of about 10%.

In one embodiment, the chitosan-derivative nanoparticle comprises chitosan coupled with glucose at a final functionalization degree of from about 2% to about 30%, from about 5% to about 30%, from about 7.5% to about 30%, from about 5% to about 25%, from about 5% to about 22%, from about 5% to about 20%, from about 5% to about 15%, or from about 5% to about 10%. In one embodiment, the chitosan-derivative nanoparticle comprises chitosan coupled with glucose at a final functionalization degree of from about 7.5% to about 25%, from about 7.5% to about 20%, from about 7.5% to about 15%, or from about 7.5% to about 12%. In one embodiment, the chitosan-derivative nanoparticle comprises chitosan coupled with glucose at a final functionalization degree of about 10%.

In one embodiment, the chitosan-derivative nanoparticle comprises chitosan coupled with cation (e.g., arginine) at a final functionalization degree of from about 2% to about 40% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 5% to about 40% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 7.5% to about 40% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 10% to about 40% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of from about 2% to about 35% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 5% to about 35% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 7.5% to about 35% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 10% to about 35% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of from about 10% to about 30% and hydrophilic polyol (e.g., glucose or gluconic acid) at a final functional degree of from about 2% to about 30%. In one embodiment, the chitosan-derivative nanoparticle comprises chitosan coupled with cation (e.g., arginine) at a final functionalization degree of from about 12% to about 30% and hydrophilic polyol (e.g., glucose or gluconic acid) at a final functional degree of from about 5% to about 25%. In one embodiment, the chitosan-derivative nanoparticle comprises chitosan coupled with cation (e.g., arginine) at a final functionalization degree of from about 14% to about 30% and hydrophilic polyol (e.g., glucose or gluconic acid) at a final functional degree of from about 7.5% to about 20%. In one embodiment, the chitosan-derivative nanoparticle comprises chitosan coupled with cation (e.g., arginine) at a final functionalization degree of from about 15% to about 30% and hydrophilic polyol (e.g., glucose or gluconic acid) at a final functional degree of from about 7.5% to about 15%, or about 10%.

In one embodiment, the chitosan-derivative nanoparticle comprises chitosan coupled with cation (e.g., arginine) at a final functionalization degree of about 25% and hydrophilic polyol (e.g., glucose or gluconic acid) at a final functional degree of from about 7.5% to about 15%. In one embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of from about 7.5% to about 15%. In one embodiment, the chitosan-derivative nanoparticle comprises chitosan coupled with cation (e.g., arginine) at a final functionalization degree of about 25% and hydrophilic polyol (e.g., glucose or gluconic acid) at a final functional degree of from about 5% to about 20%. In one embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of from about 5% to about 20%.

In a preferred embodiment, the chitosan-derivative nanoparticle comprises chitosan coupled with cation (e.g., arginine) at a final functionalization degree of about 14% and hydrophilic polyol (e.g., glucose or gluconic acid) at a final functional degree of about 10%. In a preferred embodiment, the chitosan-derivative nanoparticle comprises chitosan coupled with cation (e.g., arginine) at a final functionalization degree of about 15% and hydrophilic polyol (e.g., glucose or gluconic acid) at a final functional degree of about 12%. In another preferred embodiment, the chitosan-derivative nanoparticle comprises chitosan coupled with arginine at a final functionalization degree of about 14% and glucose at a final functional degree of about 10%. In another preferred embodiment, the chitosan-derivative nanoparticle comprises chitosan coupled with arginine at a final functionalization degree of about 15% and glucose at a final functional degree of about 12%.

In a preferred embodiment, the chitosan-derivative nanoparticle comprises chitosan coupled with cation (e.g., arginine) at a final functionalization degree of about 28% and hydrophilic polyol (e.g., glucose or gluconic acid) at a final functional degree of about 10%. In another preferred embodiment, the chitosan-derivative nanoparticle comprises chitosan coupled with arginine at a final functionalization degree of about 28% and glucose at a final functional degree of about 10%.

In some embodiments, where appropriate, DD-chitosan includes DD-chitosan derivatives, e.g., DD chitosan that incorporate an additional functionalization, e.g., DD-chitosan with an attached ligand. “Derivatives” will be understood to include the broad category of chitosan-based polymers comprising covalently modified N-acetyl-D-glucosamine and/or D-glucosamine units, as well as chitosan-based polymers incorporating other units, or attached to other moieties. Derivatives are frequently based on a modification of the hydroxyl group or the amine group of glucosamine, such as done with arginine-functionalized chitosan. Examples of chitosan derivatives include, but are not limited to, trimethylated chitosan, thiolated chitosan, galactosylated chitosan, alkylated chitosan, PEI-incorporated chitosan, uronic acid modified chitosan, glycol chitosan, and the like. For further teaching on chitosan derivatives, see, e.g., pp. 63-74 of “Non-viral Gene Therapy”, K. Taira, K. Kataoka, T. Niidome (editors), Springer-Verlag Tokyo, 2005, ISBN 4-431-25122-7; Zhu et al., Chinese Science Bulletin, December 2007, vol. 52 (23), pp. 3207-3215; and Varma et al., Carbohydrate Polymers 55 (2004) 77-93.

1.2. Chitosan Nucleic Acid Polyplex

The chitosan-derivative nanoparticle compositions generally contain at least one nucleic acid molecule, and preferably a plurality of such nucleic acid molecules. Typical nucleic acid molecules comprise phosphorous as a component of the nucleic acid backbone, e.g., in the form of a plurality of phosphodiesters or derivatives thereof (e.g., phosphorothioate). The proportion of cation-functionalized chitosan-derivative to nucleic acid can be characterized by a cation (+) to phosphorous (P) molar ratio, wherein the (+) refers to the cation of the cation-functionalized chitosan-derivative and the (P) refers to the phosphorous of the nucleic acid backbone. Typically, the (+):(P) molar ratio is selected such that the chitosan-derivative-nucleic acid complex has a positive charge in the absence of the polyanion-containing block co-polymer reversible coating. Thus, the (+):(P) molar ratio is generally greater than 1. In preferred embodiments, the (+):(P) molar ratio is greater than 1.5, at least 2, or greater than 2. In certain preferred embodiments, the (+):(P) molar ratio is greater than 2.

In some cases, the (+):(P) molar ratio is, or is about, 3:1. In some cases, the (+):(P) molar ratio is, or is about, 4:1. In some cases, the (+):(P) molar ratio is, or is about, 5:1. In some cases, the (+):(P) molar ratio is, or is about, 6:1. In some cases, the (+):(P) molar ratio is, or is about, 7:1. In some cases, the (+):(P) molar ratio is, or is about, 8:1. In some cases, the (+):(P) molar ratio is, or is about, 9:1. In some cases, the (+):(P) molar ratio is, or is about, 10:1.

In some cases, the (+):(P) molar ratio is from greater than 1 to no more than about 20:1, from about 2 to no more than about 20:1, or from about 2 to no more than about 10:1. In some cases, the (+):(P) molar ratio is from greater than about 2 to no more than about 20:1, or from greater than about 2 to no more than about 10:1. In some cases, the (+):(P) molar ratio is from about 3 to no more than about 20:1, from about 3 to no more than about 10:1, from about 3 to no more than about 8:1, or from about 3 to no more than about 7:1. In some cases, the (+):(P) molar ratio is from about 3 to no more than 20:1, from about 3 to no more than 10:1, from about 3 to no more than 8:1, or from about 3 to no more than 7:1.

In certain embodiments, the (+):(P) molar ratio is 100:1, preferably less than 100:1. For example, in certain embodiments, (+):(P) molar ratio can be from greater than 1 to less than or equal to 100:1. In some cases, the (+):(P) molar ratio can be from greater than 2 to less than or equal to 100:1. In some cases, the (+):(P) molar ratio can be from greater than or equal to 3 to less than or equal to 100:1. In some cases, the (+):(P) molar ratio can be from greater than or equal to 5 to less than or equal to 100:1. In some cases, the (+):(P) molar ratio can be from greater than or equal to 7 to less than or equal to 100:1. In some cases, the (+):(P) molar ratio can be from greater than 2 to less than or equal to 50:1. In some cases, the (+):(P) molar ratio can be from greater than or equal to 3 to less than or equal to 50:1. In some cases, the (+):(P) molar ratio can be from greater than or equal to 5 to less than or equal to 50:1. In some cases, the (+):(P) molar ratio can be from greater than or equal to 7 to less than or equal to 50:1. In some cases, the (+):(P) molar ratio can be from greater than 2 to less than or equal to 25:1. In some cases, the (+):(P) molar ratio can be from greater than or equal to 3 to less than or equal to 25:1. In some cases, the (+):(P) molar ratio can be from greater than or equal to 5 to less than or equal to 25:1. In some cases, the (+):(P) molar ratio can be from greater than or equal to 7 to less than or equal to 25:1.

In some embodiments, the cationic functional group of the chitosan-derivative nanoparticles is or comprises an amino group. Examples of such amino-functionalized chitosan-derivative nanoparticles include, but are not limited to, those containing chitosan that is functionalized with: a guanidinium or a molecule comprising a guanidinium group, a lysine, an ornithine, an arginine, or a combination thereof. In preferred embodiments, the cationic functional group is an arginine. The proportion of amino-functionalized chitosan-derivative to nucleic acid can be characterized by an amino (N) to phosphorous (P) molar ratio, wherein the (N) refers to the nitrogen atom of the amino group in the amino-functionalized chitosan-derivative and the (P) refers to the phosphorous of the nucleic acid backbone. Typically, the N:P molar ratio is selected such that the chitosan-derivative-nucleic acid complex, in the absence of PEG-PA polymer molecules, has a positive charge at a physiologically relevant pH. Thus, the N:P molar ratio is generally greater than 1. In preferred embodiments, the N:P molar ratio is greater than 1.5, at least 2, or greater than 2. In certain preferred embodiments, the N:P molar ration is greater than 2.

In some cases, the N:P molar ratio is, or is about, 3:1. In some cases, the N:P molar ratio is, or is about, 4:1. In some cases, the N:P molar ratio is, or is about, 5:1. In some cases, the N:P molar ratio is, or is about, 6:1. In some cases, the N:P molar ratio is, or is about, 7:1. In some cases, the N:P molar ratio is, or is about, 8:1. In some cases, the N:P molar ratio is, or is about, 9:1. In some cases, the N:P molar ratio is, or is about, 10:1.

In some cases, the N:P molar ratio is from greater than 1 to no more than about 20:1, from about 2 to no more than about 20:1, or from about 2 to no more than about 10:1. In some cases, the N:P molar ratio is from greater than about 2 to no more than about 20:1, or from greater than about 2 to no more than about 10:1. In some cases, the N:P molar ratio is from about 3 to no more than about 20:1, from about 3 to no more than about 10:1, from about 3 to no more than about 8:1, or from about 3 to no more than about 7:1. In some cases, the N:P molar ratio is from about 3 to no more than 20:1, from about 3 to no more than 10:1, from about 3 to no more than 8:1, or from about 3 to no more than 7:1.

In certain embodiments, the N:P molar ratio is 100:1, preferably less than 100:1. For example, in certain embodiments, N:P molar ratio can be from greater than 1 to less than or equal to 100:1. In some cases, the N:P molar ratio can be from greater than 2 to less than or equal to 100:1. In some cases, the N:P molar ratio can be from greater than or equal to 3 to less than or equal to 100:1. In some cases, the N:P molar ratio can be from greater than or equal to 5 to less than or equal to 100:1. In some cases, the N:P molar ratio can be from greater than or equal to 7 to less than or equal to 100:1. In some cases, the N:P molar ratio can be from greater than 2 to less than or equal to 50:1. In some cases, the N:P molar ratio can be from greater than or equal to 3 to less than or equal to 50:1. In some cases, the N:P molar ratio can be from greater than or equal to 5 to less than or equal to 50:1. In some cases, the N:P molar ratio can be from greater than or equal to 7 to less than or equal to 50:1. In some cases, the N:P molar ratio can be from greater than 2 to less than or equal to 25:1. In some cases, the N:P molar ratio can be from greater than or equal to 3 to less than or equal to 25:1. In some cases, the N:P molar ratio can be from greater than or equal to 5 to less than or equal to 25:1. In some cases, the N:P molar ratio can be from greater than or equal to 7 to less than or equal to 25:1.

In a preferred embodiment, the subject polyplexes have amine to phosphate (N/P) ratio of 2 to 100, e.g., 2 to 50, e.g., 2 to 40, e.g., 2 to 30, e.g., 2 to 20, e.g., 2 to 5. Preferably, the N/P ratio is inversely proportional to the molecular weight of the chitosan, i.e., a smaller molecular weight (e.g., dually) derivatized-chitosan requires a higher N/P ratio, and vice versa.

A nucleic acid of the present invention will generally contain phosphodiester bonds, although in some cases nucleic acid analogs are included that may have alternate backbones or other modifications or moieties incorporated for any of a variety of purposes, e.g., stability and protection. Other analog nucleic acids contemplated include those with non-ribose backbones. In addition, mixtures of naturally occurring nucleic acids, analogs, and both can be made. The nucleic acids may be single stranded or double stranded or contain portions of both double stranded or single stranded sequence. Nucleic acids include but are not limited to DNA, RNA and hybrids where the nucleic acid contains any combination of deoxyribo- and ribo-nucleotides, and any combination of bases, including uracil, adenine, thymine, cytosine, guanine, inosine, xanthanine, hypoxanthanine, isocytosine, isoguanine, etc. Nucleic acids include DNA in any form, RNA in any form, including triplex, duplex or single-stranded, anti-sense, siRNA, ribozymes, deoxyribozymes, polynucleotides, oligonucleotides, chimeras, microRNA, and derivatives thereof. Nucleic acids include artificial nucleic acids, including but not limited to, peptide nucleic acid (PNA), phosphorodiamidate morpholino oligo (PMO), locked nucleic acid (LNA), glycol nucleic acid (GNA) and threose nucleic acid (TNA). It will be appreciated that, for artificial nucleic acids that do not comprise phosphorous, an equivalent measure of the (+):P or N:P ratio can be approximated by the number of nucleotide (or nucleotide analog) bases.

In a preferred embodiment, the polyplexes of the compositions comprise chitosan molecules having an average molecular weight of less than 110 kDa, more preferably less than 65 kDa, more preferably less than 50 kDa, more preferably less than 40 kDa, and most preferably less than 30 kDa before functionalization. In some embodiments, polyplexes of the compositions comprise chitosan having an average molecular weight of less than 15 kDa, less than 10 kDa, less than 7 kDa, or less than 5 kDa before functionalization.

In a preferred embodiment, the polyplexes comprise chitosan molecules having on average less than 680 glucosamine monomer units, more preferably less than 400 glucosamine monomer units, more preferably less than 310 glucosamine monomer units, more preferably less than 250 glucosamine monomer units, and most preferably less than 190 glucosamine monomer units. In some embodiments, the polyplexes comprise chitosan molecules having on average less than 95 glucosamine monomer units, less than 65 glucosamine monomer units, less than 45 glucosamine monomer units, or less than 35 glucosamine monomer units.

Chitosan, and (e.g., dually) derivatized-chitosan nucleic acid polyplexes may be prepared by any method known in the art, including but not limited to those described herein.

1.2.1. Nucleic Acids

As described above, the chitosan polyplexes can contain a plurality of nucleic acids. In one embodiment, the nucleic acid component comprises a therapeutic nucleic acid. The subject (e.g., dually) derivatized-chitosan nucleic acid polyplexes are amenable to the use of any therapeutic nucleic acid known in the art including, e.g., nucleic acids encoding therapeutic proteins such as hormones, enzymes, cytokines, chemokines, antibodies, mitogenic factors, growth factors, differentiation factors, factors influencing cell apoptosis, factors influencing inflammation, factors influencing the immune response (e.g. immunostimulators), and the like.

A therapeutic nucleic acid may be used to effect genetic therapy by serving as a replacement or enhancement for a defective gene or to compensate for lack of a particular gene product, by encoding a therapeutic product. A therapeutic nucleic acid may also inhibit expression of an endogenous gene. A therapeutic nucleic acid may encode all or a portion of a translation product, and may function by recombining with DNA already present in a cell, thereby replacing a defective portion of a gene. It may also encode a portion of a protein and exert its effect by virtue of co-suppression of a gene product.

In some embodiments, the nucleic acid component comprises a therapeutic nucleic acid construct. The therapeutic nucleic acid construct is a nucleic acid construct capable of exerting a therapeutic effect. Therapeutic nucleic acid constructs may comprise nucleic acids encoding therapeutic proteins, as well as nucleic acids that produce transcripts that are therapeutic RNAs.

In the preferred embodiments described and exemplified herein, the therapeutic nucleic acid construct comprises a nucleic acid encoding IL-12, either alone or in conjunction with an additional immunostimulatory molecule(s) IL-12 is a heterodimeric type 1 cytokine with a four α-helical bundle structure. The active heterodimer, also known as IL-12 p70, comprises 2 subunits encoded by two separate genes, IL-12A (encoding p35) and IL-12B (encoding p40). There are at least 6 splice variant transcripts of IL-12A (ENST00000305579.6, ENST00000466512.1, ENST00000480787.5, ENST00000468862.5, ENST00000496308.1, and ENST00000480088.1). Nucleic and peptide sequences for the human IL-12A isoform 1 precursor are, for example, NM_000882.4, NM_001354582.2, NM_001354583.2, and NP_000873.2, NP_001341511.1, and NP_001341512.1 respectively. Mouse IL12a nucleic and peptide sequences are, for example, NM_001159424.2 and NP_001152896.1, respectively. Human IL-12B genomic sequence, transcript, and peptide sequences are, for example, NG_009618.1, NM_002187.3, and NP_002178.2, respectively. Mouse IL-12B nucleic and peptide sequences are for example, NM_001303244 and NP_001290173.1.

In some embodiments, the single chain IL-12 protein can be generated by fusing the p40 subunit to the p35 subunit through a short amino acid linker sequence. The two subunits can be linked in either the p40-linker-p35 or p35-linker-p40 orientation. The protein can be secreted as a result of the inclusion of the signal peptide from the subunit 5′ of the linker, while the signal peptide is removed from the subunit downstream of the linker sequence. In preferred embodiments, the linker sequence comprises a 10 amino acid sequence derived from bovine elastin and comprised of valine (V), proline (P) and glycine (G) residues (VPGVGVPGVG). In some embodiments, the linker sequence may contain G and/or serine (S) residues, such as (GGGGS)n. In other embodiments, the linker sequence may contain G, S and additional amino acids, including but not limited to, P, arginine (R), lysine (K), threonine (T) and glutamic acid (E). In exemplary embodiments the linker is selected from the group consisting of GSGSSRGGSGSGGSGGGGSK, GSTSG(A/S)GKSSEGKG (SEQ ID NO:1), GSTSGSGKPGSGEGSTKG (SEQ ID NO:2), GGGGGGS (SEQ ID NO:3), or GGGGSGGGGSGGGGS (SEQ ID NO:4).

In an exemplary embodiment, the nucleic acid sequence encoding hIL-12p40p35 comprises:

(SEQ ID NO: 5)
atgtgccatcagcaacttgtcatctcctggttctccctcgtgttcctggc
ctcccctcttgtcgcgatttgggagctgaagaaagatgtgtacgtcgtgg
aactcgactggtacccggacgcccccggggaaatggtggtgctcacttgt
gatactcccgaagaggatggaattacctggaccctcgatcagtcctccga
ggtcttgggatccggcaaaactctgaccatccaagtcaaggaattcggcg
acgcggggcagtacacctgtcacaagggcggagaagtgctgtcgcactca
ctcctgctccttcacaaaaaggaggacggcatctggtcgaccgacatcct
gaaggaccagaaggaacccaagaacaagacctttctgcgctgcgaggcca
agaactattcgggaaggttcacctgttggtggctgactaccatctccacc
gacctgactttctccgtgaagtcctctcggggttcgagcgacccgcaggg
tgttacgtgcggtgctgcaaccctgtccgcggagagagtgcggggggaca
acaaggaatacgagtactcagtggaatgccaggaagatagcgcctgccct
gccgccgaagagtccctgccgattgaagtcatggtggacgcagtgcataa
gttgaaatatgagaactacacctcgtcgttcttcatccgggacatcatca
agcctgacccccctaagaatctgcagctcaagcccctcaagaactccaga
caggtcgaagtgtcctgggagtacccagatacgtggagcacaccgcactc
gtacttctccttgaccttctgcgtccaagtgcagggaaagtccaaacggg
agaagaaggaccgcgtgttcactgataagacttccgctactgtgatctgc
cgcaaaaacgccagcatcagcgtgcgcgcgcaagatagatactactcaag
ctcttggtccgaatgggcgtccgtgccatgctcggtgcccggcgtgggcg
tgcctggagtgggagcccggaacttgccggtggccacccctgaccccgga
atgttcccttgcctgcaccactcccaaaaccttctgagggctgtgtccaa
catgctgcagaaggctcggcagaccctggaattctacccctgcacctccg
aggagatcgaccacgaagatattaccaaggacaagacctcaaccgtggaa
gcctgcctgcccctggaactgaccaagaacgaatcgtgcctgaatagccg
ggaaacctccttcatcaccaacggctcctgcctggcctcacgaaagacca
gctttatgatggccctgtgcctgagctcgatctacgaggacctgaagatg
taccaggtcgagttcaagactatgaacgccaagctgctgatggatccgaa
gcggcagatcttcttggaccagaatatgctggcagtgatcgacgagctga
tgcaggccctcaacttcaactccgagactgtgccgcaaaagtcgagcctg
gaggaaccggacttctacaagaccaagatcaagttatgtattctcctgca
cgcgtttaggattcgcgccgtgaccattgatagagtgatgtcctacctga
acgccagctga.

In an exemplary embodiment, the hIL-12p40p35 amino acid sequence comprises:

(SEQ ID NO: 6)
M C H Q Q L V I S W F S L V F L A S P L V A I W E
L K K D V Y V V E L D W Y P D A P G E M W L T C D
T P E E D G I T W T L D Q S S E V L G S G K T L T
I Q V K E F G D A G Q Y T C H K G G E V L S H S L
L L L H K K E D G I W S T D I L K D Q K E P K N K
T F L R C E A K N Y S G R F T C W W L T T I S T D
L T F S V K S S R G S S D P Q G V T C G A A T L S
A E R V R G D N K E Y E Y S V E C Q E D S A C P A
A E E S L P I E V M V D A V H K L K Y E N Y T S S
F F I R D I I K P D P P K N L Q L K P L K N S R Q
V E V S W E Y P D T W S T P H S Y F S L T F C V Q
V Q G K S K R E K K D R V F T D K T S A T V I C R
K N A S I S V R A Q D R Y Y S S S W S E W A S V P
C S V P G V G V P G V G A R N L P V A T P D P G M
F P C L H H S Q N L L R A V S N M L Q K A R Q T L
E F Y P C T S E E I D H E D I T K D K T S T V E A
C L P L E L T K N E S C L N S R E T S F I T N G S
C L A S R K T S F M M A L C L S S I Y E D L K M Y
Q V E F K T M N A K L L M D P K R Q I F L D Q N M
L A V I D E L M Q A L N F N S E T V P Q K S S L E
E P D F Y K T K I K L C I L L H A F R I R A V T I
D R V M S Y L N A S Stop.

Therapeutic nucleic acids also include therapeutic DNA in the form of a circular double-stranded DNA plasmid, minicircle DNA (Science Report 6:2315, 2016) or closed-ended linear duplex DNA (Li et al, PLoS One 8(8): e69879, 2013).

Therapeutic nucleic acids also include therapeutic RNAs, which are RNA molecules capable of exerting a therapeutic effect in a mammalian cell. Therapeutic RNAs include, but are not limited to, messenger RNAs, antisense RNAs, siRNAs, short hairpin RNAs, micro RNAs, and enzymatic RNAs. Therapeutic nucleic acids include, but are not limited to, nucleic acids intended to form triplex molecules, protein binding nucleic acids, ribozymes, deoxyribozymes, and small nucleotide molecules. Many types of therapeutic RNAs are known in the art. For example, see Meng et al., A new developing class of gene delivery: messenger RNA-based therapeutics, Biomater. Sci., 5, 2381-2392, 2017; Grimm et al., Therapeutic application of RNAi is mRNA targeting finally ready for prime time? J. Clin. Invest., 117:3633-3641, 2007; Aagaard et al., RNAi therapeutics: Principles, prospects and challenges, Adv. Drug Deliv. Rev., 59:75-86, 2007; Dorsett et al., siRNAs: Applications in functional genomics and potential as therapeutics, Nat. Rev. Drug Discov., 3:318-329, 2004. These include double-stranded short interfering RNA (siRNA).

1.2.1.1. Expression Control Regions

In a preferred embodiment, a polyplex of the invention comprises a therapeutic nucleic acid, which is a therapeutic construct, comprising an expression control region operably linked to a coding region. The therapeutic construct produces therapeutic nucleic acid, which may be therapeutic on its own, or may encode a therapeutic protein.

In some embodiments, the expression control region of a therapeutic construct possesses constitutive activity. In a number of preferred embodiments, the expression control region of a therapeutic construct does not have constitutive activity. This provides for the dynamic expression of a therapeutic nucleic acid. By “dynamic” expression is meant expression that changes over time. Dynamic expression may include several such periods of low or absent expression separated by periods of detectable expression. In a number of preferred embodiments, the therapeutic nucleic acid is operably linked to a regulatable promoter. This provides for the regulatable expression of therapeutic nucleic acids.

Expression control regions comprise regulatory polynucleotides (sometimes referred to herein as elements), such as promoters and enhancers, which influence expression of an operably linked therapeutic nucleic acid.

Expression control elements included herein can be from bacteria, yeast, plant, or animal (mammalian or non-mammalian). Expression control regions include full-length promoter sequences, such as native promoter and enhancer elements, as well as subsequences or polynucleotide variants that retain all or part of full-length or non-variant function (e.g., retain some amount of nutrient regulation or cell/tissue-specific expression). As used herein, the term “functional” and grammatical variants thereof, when used in reference to a nucleic acid sequence, subsequence or fragment, means that the sequence has one or more functions of native nucleic acid sequence (e.g., non-variant or unmodified sequence). As used herein, the term “variant” means a sequence substitution, deletion, or addition, or other modification (e.g., chemical derivatives such as modified forms resistant to nucleases).

As used herein, the term “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Some expression control regions confer regulatable expression to an operably linked therapeutic nucleic acid. A signal (sometimes referred to as a stimulus) can increase or decrease expression of a therapeutic nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

Numerous regulatable promoters are known in the art. Preferred inducible expression control regions include those comprising an inducible promoter that is stimulated with a small molecule chemical compound. In one embodiment, an expression control region is responsive to a chemical that is orally deliverable but not normally found in food. Particular examples can be found, for example, in U.S. Pat. Nos. 5,989,910; 5,935,934; 6,015,709; and 6,004,941.

Promoter/enhancer sequences of particular interest include:

| Promoter/ | |
|---|---|
| enhancer | |
| sequence | Description |
| CMV | Cytomegalovirus immediate early enhancer and promoter |
| EF1α | Human elongation factor (EF)-1α promoter |
| CMV/EF1α | CMV enhancer + core EF1α promoter |
| 2 x CMV/ | 2 x CMV enhancer + core EF1α promoter |
| EF1α | |
| CAG | CMV enhancer + promoter, first exon and first intron of |
| | chicken beta-actin gene + splice acceptor of the rabbit |
| | beta globin gene |
| CMV/ | CMV enhancer + Human elongation factor (EF)-1α |
| EF1α/ | promoter + R segment and part of U5 sequence (R′-U5) |
| HTLV | of human T-cell leukemia virus Type 1 Long Terminal |
| | Repeat |

In some embodiments of the invention, the therapeutic construct is comprised within a plasmid comprising an origin, a multicloning site and a selectable marker. In some embodiments, plasmids of less than 10 kb are desirable. In some embodiments the plasmids used are suitable for gene therapy in human patients, and/or are engineered for high levels of transient gene expression in mammalian tissues. In preferred embodiments, the plasmid is selected from the group consisting of the Nanoplasmid™ (e.g. NTC9385 plasmid, NTC9385R, NTC9385R-RIG-I, NTC9385R (3 CpG), NTC9385R-eRNA41H-CpG, NTC8685 plasmid (Nature Technology), gWIZ plasmid (Genlantis), or pVAX1 plasmid (Thermofisher Scientific). See, e.g., U.S. Pat. Nos. U.S. Pat. Nos. 6,027,722, 6,287,863, 6,410,220, 6,573,091, 9,012,226, 9,017,966, 9,018,012, 9,109,012, 9,487,788, 9,487,789, 9,506,082, 9,550,998, 9,725,725, 9,737,620, 9,950,081, 10,047,365, 10,144,935, and 10,167,478. In some embodiments, the plasmid has been “retrofitted” to remove antibiotic selection agents and/or to increase expression levels.

For further teaching, see WO 2008/020318, which is expressly incorporated herein in its entirety by reference. In one embodiment, the nucleic acid of the (e.g., dually) derivatized-chitosan nucleic acid polyplex is an artificial nucleic acid.

In one embodiment, the nucleic acid of the DD-chitosan nucleic acid polyplex is a therapeutic nucleic acid. In one embodiment, the therapeutic nucleic acid is a therapeutic RNA. Preferred therapeutic RNAs include, but are not limited to, antisense RNA, siRNA, short hairpin RNA, micro RNA, and enzymatic RNA.

In one embodiment, the therapeutic nucleic acid is DNA.

In one embodiment, the therapeutic nucleic acid comprises a nucleic acid sequence encoding a therapeutic protein.

1.3. Polyols

Chitosan-derivative nanoparticles can be functionalized with a polyol. Polyols useful in the present invention in general are typically hydrophilic. In some cases, the chitosan-derivative nanoparticles are functionalized with a cationic component such as an amino group and with a polyol. Such chitosan-derivative nanoparticles functionalized with a cationic moiety such as an amino group and a polyol are referred to as “dually-derivatized chitosan nanoparticles.”

In some embodiments, the chitosan-derivative nanoparticle comprises a polyol of Formula II:

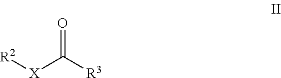

wherein:
R² is selected from: H and hydroxyl;
R³ is selected from: H and hydroxyl; and
X is selected from: C₂-C₆ alkylene optionally substituted with one or more hydroxyl substituents.

In some embodiments, the chitosan-derivative nanoparticle is functionalized with a polyol of Formula II, wherein R² is selected from: H and hydroxyl; R³ is selected from: H and hydroxyl; and X is selected from: C₂-C₆ alkylene optionally substituted with one or more hydroxyl substituents.

In some embodiments, the chitosan-derivative nanoparticle comprises a polyol of Formula III:

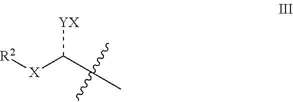

wherein: [symbol]
is ═O or —H₂;
R² is selected from: H and hydroxyl;
R³ is selected from: H and hydroxyl;
X is selected from: C₂-C₆ alkylene optionally substituted with one or more hydroxyl substituents; and

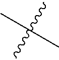

denotes the bond between the polyol and the derivatized chitosan.

In one embodiment, a polyol according to the present invention having 3 to 7 carbons may have one or more carbon-carbon multiple bonds. In a preferred embodiment, a polyol according to the present invention comprises a carboxyl group. In a further preferred embodiment, a polyol according to the present invention comprises an aldehyde group. A skilled artisan will recognize that when a polyol according to the present invention comprises an aldehyde group, such polyol encompasses both the open-chain conformation (aldehyde) and the cyclic conformation (hemiacetal).

Non-limiting examples of a polyols include gluconic acid, threonic acid, glucose and threose. Examples of other such polyols, which may have a carboxyl and/or aldehyde group, or may be a saccharide or acid form thereof, are described in more detail in U.S. Pat. No. 10,046,066, the disclosure of which is expressly incorporated by reference herein. A skilled artisan will recognize that the polyols are not limited to a specific stereochemistry.

In a preferred embodiment, the polyol may be selected from the group consisting of 2,3-dihydroxylpropanoic acid; 2,3,4,5,6,7-hexahydroxylheptanal; 2,3,4,5,6-pentahydroxylhexanal; 2,3,4,5-tetrahydroxylhexanal; and 2,3-dihydroxylpropanal.

In a preferred embodiment, the polyol may be selected from the group consisting of D-glyceric acid, L-glyceric acid, L-glycero-D-mannoheptose, D-glycero-L-mannoheptose, D-glucose, L-glucose, D-fucose, L-fucose, D-glyceraldehyde, and L-glyceraldehyde.

In some embodiments, the polyol may be compound of Formula IV or Formula V:

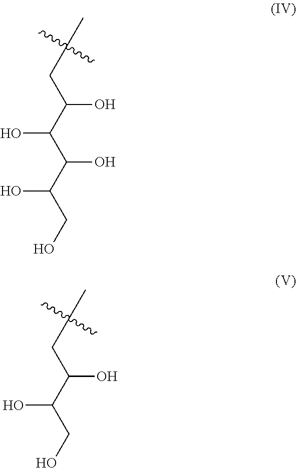 [SMILES: *CC(O)C(O)C(O)C(O)CO ; *CC(O)C(O)CO]

In a preferred embodiment, the polyol is a compound of Formula IV. In some cases, the polyol of Formula IV has been coupled to the chitosan by reductive amination.

A hydrophilic polyol that has a carboxyl group may be coupled to chitosan or a cation functionalized chitosan such as an amine-functionalized chitosan (e.g., Arg-coupled chitosan (Arg-chitosan)). In some embodiments, the polyol is coupled at a reaction pH of 6.0±0.3. At this pH, the carboxylic acid group of the hydrophilic polyol may be attacked by uncoupled amines on the chitosan backbone according to a nucleophilic substitution reaction mechanism.

An ordinarily skilled artisan will recognize that, when coupling such a hydrophilic polyol to Arg-chitosan, it is also possible that a small amount of the hydrophilic polyol may form a covalent bond with an amine group of the Arg through the same mechanism, although it is likely that the nucleophilic substitution reaction will occur predominantly with the amine group of the chitosan backbone.

A hydrophilic polyol that is a natural saccharide may be coupled to chitosan, cation-functionalized chitosan, such as amine-functionalized chitosan (e.g., Arg-coupled chitosan (Arg-chitosan)) using reductive amination followed by reduction with NaCBH₃ or NaBH.

1.4. Polymer:Polyplex Compositions

Chitosan polyplexes can be mixed with a plurality of polymers, the polymers comprising a hydrophilic, non-charged portion, and a negatively charged (anionic) portion. As described above, the chitosan polyplexes are formulated to have a positive charge in the absence of, or prior to, complexing with the anionic portion-containing polymer. Thus under suitable conditions, the polymer component will form a reversible charge:charge complex with the chitosan-derivative nucleic acid polyplexes. In some embodiments, the polymers of the polymer component are unbranched. In some embodiments, the polymers are branched. In some cases, the polymer component comprises a mixture of branched and unbranched polymers.

In some embodiments, the polymer component is released from the chitosan polyplex after administration, after entering a cell, and/or after endocytosis. Without wishing to be bound by theory, it is hypothesized that the polyplex:polymer compositions thus formed by complexing polyplex and the anionic portion-containing polymer can provide improved in vitro, in solution, and/or in vivo stability without substantially interfering with transfection efficiency. In some embodiments, the polyplex:polymer compositions thus formed can provide reduced mucoadhesive properties as compared to, e.g., otherwise identical, polyplexes without the polymer component.

In a preferred embodiment, the polyplex:polymer compositions have a low net positive, neutral, or net negative zeta potential (from about +10 mV to about −20 mV) at physiological pH. Such compositions can exhibit reduced aggregation in physiological conditions and reduced non-specific binding to ubiquitous anionic components in vivo. Said properties can enhance migration of such composition (e.g., enhanced diffusion in mucus) to contact the cell and result in enhanced intracellular release of nucleic acid.

In a preferred embodiment, the polyplex:polymer particle compositions have an average hydrodynamic diameter of less than 1000 nm, more preferably less than 500 nm and most preferably less than 200 nm. In certain embodiments, the polyplex:polymer particle compositions have an average hydrodynamic diameter of from 50 nm to no more than 1000 nm, preferably from 50 nm to no more than 500 nm and most preferably from 50 nm to no more than 200 nm. In certain embodiments, the polyplex:polymer particle compositions have an average hydrodynamic diameter of from 50 nm to no more than 175 nm, preferably from 50 nm to no more than 150 nm. In certain embodiments, the polyplex:polymer particle compositions have an average hydrodynamic diameter of from 75 nm to no more than 1000 nm, preferably from 75 nm to no more than 500 nm and most preferably from 75 nm to no more than 200 nm. In certain embodiments, the polyplex:polymer particle compositions have an average hydrodynamic diameter of from 75 nm to no more than 175 nm, preferably from 75 nm to no more than 150 nm. In certain embodiments, the polyplex:polymer particle compositions have an average hydrodynamic diameter of greater than 100 nm and less than 175 nm.

In one embodiment, the polyplex:polymer compositions have a % supercoiled DNA content of 80%, at least 80%, or preferably 90%, more preferably at least 90%.

In one embodiment, the polyplex:polymer compositions have an average zeta potential of between +10 mV to −10 mV at a physiological pH, most preferably between +5 mV to −5 mV at a physiological pH.

The polyplex:polymer compositions are preferably homogeneous in respect of particle size. Accordingly, in a preferred embodiment, the composition has a low average polydispersity index (“PDI”). In an especially preferred embodiment, a dispersion of the polyplex:polymer composition has a PDI of less than 0.5, more preferably less than 0.4, more preferably less than 0.3, yet more preferably less than 0.25, and most preferably less than 0.2.

In some cases, a dispersion of the polyplex:polymer composition exhibits one or more of the foregoing PDI, average zeta potential, % supercoil DNA, or average particle size (nm) or size range after one or more freeze thaw cycles. In some cases, a dispersion of the polyplex:polymer composition exhibits one or more of the foregoing PDI, average zeta potential, % supercoil DNA, or average particle size (nm) or size range after storage in solution for at least 48 h at 4° C. In some cases, a dispersion of the polyplex:polymer composition exhibits one or more of the foregoing PDI, average zeta potential, % supercoil DNA, or average particle size (nm) or size range after storage in solution for at least 1 or 2 weeks, or more at 4° C.

In some cases, a dispersion of the polyplex:polymer composition exhibits one or more of the foregoing PDI, average zeta potential, % supercoil DNA, or average particle size (nm) or size range after lyophilization and rehydration. In some cases, a dispersion of the polyplex:polymer composition exhibits one or more of the foregoing PDI, average zeta potential, % supercoil DNA, or average particle size (nm) or size range after spray drying and rehydration. In some cases, a dispersion of the polyplex:polymer composition exhibits one or more of the foregoing PDI, average zeta potential, % supercoil DNA, or average particle size (nm) or size range when concentrated (e.g., by ultrafiltration such as tangential flow filtration) to a nucleic acid concentration of at least 250 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 1,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 25,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 2,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 5,000 μg/mL. In some cases, a dispersion of the polyplex:polymer composition exhibits one or more of the foregoing PDI, average zeta potential, % supercoil DNA, or average particle size (nm) or size range when concentrated to a nucleic acid concentration of from 125 μg/mL to about 10,000 μg/mL.

In general, the polyplex:polymer compositions described herein, exhibit favorable solution behavior (e.g., stability and/or non-aggregation) as measured by PDI or mean particle size even in the absence of excipients such as lyoprotectants, cryoprotectants, surfactants, rehydration or wetting agents, and the like. In some cases, the polyplex:polymer compositions described herein exhibit favorable solution behavior (e.g., stability and/or non-aggregation) as measured by PDI or mean particle size in physiological fluids or simulated physiological fluids. For example, in some embodiments, the polyplex:polymer compositions described herein are stable in simulated intestinal fluid, in mammalian urine, and/or when stored in a mammalian bladder (e.g., and in contact with urine).

As described above, the polyplex:polymer compositions described herein are preferably substantially size stable in the composition. In a preferred embodiment, a composition of the invention comprises polyplex:polymer particles that increase in average diameter by less than 100%, more preferably less than 50%, and most preferably less than 25%, at room temperature for 6 hours, more preferably 12 hours, more preferably 24 hours, and most preferably 48 hours. In a particularly preferred embodiment, a composition of the invention comprises polyplex:polymer particles that increase in average diameter by less than 25% at room temperature for at least 24 hours or at least 48 hours.

The polyplex:polymer particles of the subject compositions are preferably substantially size stable under cooled conditions. In a preferred embodiment, a composition of the invention comprises polyplex:polymer particles that increase in average diameter by less than 100%, more preferably less than 50%, and most preferably less than 25%, at 2-8 degrees Celsius for 6 hours, more preferably 12 hours, more preferably 24 hours, and most preferably 48 hours.

The polyplex:polymer particles of the subject compositions are preferably substantially size stable under freeze-thaw conditions. In a preferred embodiment, a composition of the invention comprises polyplexes that increase in average diameter by less than 100%, more preferably less than 50%, and most preferably less than 25% at room temperature for 6 hours, more preferably 12 hours, more preferably 24 hours, and most preferably 48 hours following thaw from frozen at −20 to −80 degrees Celsius.

In a preferred embodiment, the composition has a nucleic acid concentration greater than 0.5 mg/ml, and is substantially free of precipitated polyplex. More preferably, the composition has a nucleic acid concentration of at least 0.6 mg/ml, more preferably at least 0.75 mg/ml, more preferably at least 1.0 mg/ml, more preferably at least 1.2 mg/ml, and most preferably at least 1.5 mg/ml, and is substantially free of precipitated polyplex. In another preferred embodiment, the composition has a nucleic acid concentration greater than 2 mg/ml, and is substantially free of precipitated polyplex. More preferably, the composition has a nucleic acid concentration of at least 2.5 mg/ml, more preferably at least 5 mg/ml, more preferably at least 10 mg/ml, more preferably at least 15 mg/ml, and most preferably about 25 mg/ml, and is substantially free of precipitated polyplex. In some embodiments, the composition has a nucleic acid concentration from 0.5 mg/mL to about 25 mg/mL, and is substantially free of precipitated polyplex. In some embodiments, the composition has a nucleic acid concentration of ≤ about 25 mg/mL, and is substantially free of precipitated polyplex. The compositions can be hydrated. In a preferred embodiment, the composition is substantially free of uncomplexed nucleic acid.

In a preferred embodiment, the polyplex:polymer particle composition is isotonic. Achieving isotonicity, while maintaining polyplex stability, is highly desirable in formulating pharmaceutical compositions, and these preferred compositions are well suited to pharmaceutical formulation and therapeutic applications.

In certain embodiments, the polyplex:polymer particle composition can be uncoated to release all or part of the, e.g., PEG, polymer coat by reducing pH. In certain embodiments, the polymer coat is released by incubating the particle under a pH condition that is below the pKa of the polyanionic anchor region of the polymer. For example, where the polymer coat is polyglutamate, the polymer coat can be released by incubating the particle at a pH below the pKa of polyglutamate, such as a pH of less than about 4.25. In certain embodiments, the polymer coat can be released by incubating the particle under a pH condition that is at least 0.25 pH units or at least 0.5 pH units below the pKa of the polyanion anchor region of the polymer coat.

In certain embodiments, the polyplex:polymer particle composition can be uncoated to release all or part of the, e.g., PEG, polymer coat by subjecting the particle to a high ionic strength.

Without wishing to be bound by theory, it is hypothesized that certain physiological conditions can promote partial (e.g. >5%), substantial (>50%), extensive, (e.g., >90%), or complete (100%) uncoating of reversibly PEGylated chitosan DNA polyplexes described herein. For example, low pH conditions in certain subcellular compartments (e.g., endosome, early endosome, late endosome, or lysosome) can facilitate release of the polymer coat. As another example, certain extracellular conditions can promote partial (e.g., >5%), substantial (>50%), extensive (>90%), or complete (100%) uncoating of reversibly PEGylated chitosan DNA polyplexes described herein. In some cases, the high ionic strength and/or acidic pH conditions typically encountered in certain positions in the alimentary canal can promote partial (e.g. >5%), substantial (>50%), extensive (>90%), or complete (100%) uncoating of reversibly PEGylated chitosan DNA polyplexes described herein.

In certain embodiments, PEGylated polyplexes described herein are formulated for delivery to a cell, tissue, or bodily compartment (e.g., intestine, small intestine, large intestine, colon, lung, or bladder) such that the polyplexes remain PEGylated and thereby facilitate transfection of the target cell. In some embodiments, PEGylated polyplexes described herein partially (e.g. >5%), substantially (>50%), extensively (e.g., >90%), or completely (100%) release the polymer coat after or during entry into the intracellular environment. In certain embodiments, PEGylated polyplexes described herein are formulated for delivery to a cell, tissue or bodily compartment (e.g., intestine, small intestine, large intestine, colon, lung, or bladder) such that the PEGylated polyplexes described herein partially (e.g., >5%), substantially (>50%), extensively (e.g., >90%), or completely (100%) release the polymer coat upon delivery to a cell, tissue or bodily compartment (e.g., intestine, small intestine, large intestine, colon, lung or bladder).

It will be appreciated that anion charge density and/or pKa of the anionic anchor region of a polymer can be adjusted to promote or inhibit release under intended conditions. It will similarly be appreciated that the pH, volume, and ionic strength, and other conditions of the formulation can be adjusted to promote or inhibit release under intended conditions. For example, for delivery to the intestine through the low pH gastric environment, a PEGylated polyplex formulation can be enteric coated and/or delivered in a buffering agent to increase the pH of the gastric environment. Optimized reversibly PEGylated particle compositions can be identified by assaying for stability and transfection efficiency using assays described herein.

The compositions comprising chitosan polyplex complexed with the anionic portion-containing polymer can be characterized by the ratio of cationic functional groups of the (e.g., dually) derivatized-chitosan polyplex (+) to anion moieties of the polymer (−), referred to as the “(+):(−) molar ratio”. This (+):(−) molar ratio can vary from greater than about 1:100 to less than about 10:1.

In certain embodiments, the (+):(−) molar ratio can be from greater than about 1:75 to less than about 8:1. In some cases, the (+):(−) molar ratio can be from greater than 1:10 to less than 10:1. In some cases, the (+):(−) molar ratio can be from, or from about, 1:10 to, or to about, 10:1. In some cases, the (+):(−) molar ratio can be from, or from about, 1:8 to, or to about, 8:1. In certain embodiments, the (+):(−) molar ratio can be from greater than 1:50 to less than about 10:1. In some cases, the (+):(−) molar ratio can be from greater than 1:25 to less than about 10:1. In some cases, the (+):(−) molar ratio can be from greater than 1:10 to less than about 7:1. In some cases, the (+):(−) molar ratio can be from greater than 1:8 to less than about 7:1. In some cases, the (+):(−) molar ratio can be from greater than 1:8 to less than about 6:1.

In certain embodiments, where the cationic functional group of the (e.g., dually) derivatized-chitosan polyplex is an amino moiety, the compositions comprising chitosan polyplex complexed with the anionic portion-containing polymer can be characterized by the ratio of amino groups of the (e.g., dually) derivatized-chitosan polyplex (N) to anion (A) moieties of the polymer, referred to as the “N:A molar ratio”. This N:A molar ratio can vary from greater than about 1:100 to less than about 10:1.

In certain embodiments, the N:A molar ratio can be from greater than about 1:75 to less than about 8:1. In some cases, the N:A molar ratio can be from greater than 1:10 to less than 10:1. In some cases, the N:A molar ratio can be from, or from about, 1:10 to, or to about, 10:1. In some cases, the N:A molar ratio can be from, or from about, 1:8 to, or to about, 8:1. In certain embodiments, the N:A molar ratio can be from greater than 1:50 to less than about 10:1. In some cases, the N:A molar ratio can be from greater than 1:25 to less than about 10:1. In some cases, the N:A molar ratio can be from greater than 1:10 to less than about 7:1. In some cases, the N:A molar ratio can be from greater than 1:8 to less than about 7:1. In some cases, the N:A molar ratio can be from greater than 1:8 to less than about 6:1.

Additionally or alternatively, the compositions comprising chitosan polyplex complexed with the anionic portion-containing polymer can be characterized by a three-component ratio of cationic functional groups of the (e.g., dually) derivatized-chitosan polyplex (+) to phosphorus atoms of the nucleic acid (P) to anion moieties of the polymer (−), referred to as the “(+):P:(−) molar ratio”.

In certain embodiments, where (+):P is from at least 2:1 to no more than 20:1, the molar ratio of (+):(−) can vary from at least 1:40 to about 40:1. In certain embodiments, where (+):P is from at least 2:1 to no more than 20:1, the molar ratio of (+):(−) can vary from at least 1:40 to about 1:10. In some embodiments, where (+):P is from at least 2:1 to no more than 20:1, the molar ratio of (+):(−) can vary from at least 1:25 to about 25:1. In some embodiments, where (+):P is from at least 2:1 to no more than 20:1, the molar ratio of (+):(−) can vary from at least 1:25 to about 1:10. In some cases, where (+):P is from at least 2:1 to no more than 20:1, the molar ratio of (+):(−) can vary from at least 1:20 to about 20:1. In some cases, where (+):P is from at least 2:1 to no more than 20:1, the molar ratio of (+):(−) can vary from at least 1:20 to about 1:10. In some cases, where (+):P is from at least 2:1 to no more than 20:1, the molar ratio of (+):(−) can vary from at least 1:10 to about 10:1. In some cases, where (+):P is from at least 2:1 to no more than 20:1, the molar ratio of (+):(−) can vary from at least 1:25 to about 2:1. In some cases, where (+):P is from at least 2:1 to no more than 20:1, the molar ratio of (+):(−) can vary from at least 1:20 to about 1:1.

In certain preferred embodiments, (+):P:(−) is from 3:1:3.5 to 3:1:17.5. In certain preferred embodiments, (+):P:(−) is from 5:1:3.5 to 5:1:17.5. In certain preferred embodiments, (+):P:(−) is from 7:1:3.5 to 7:1:17.5. In certain preferred embodiments, (+):P:(−) is about 3:1:3.5, 3:1:7, 3:1:10, 3:1:15, 3:1:17.5, or 3:1:20. In certain preferred embodiments, (+):P:(−) is about 5:1:3.5, 5:1:7, 5:1:10, 5:1:15, 5:1:17.5, or 5:1:20. In certain preferred embodiments, (+):P:(−) is about 7:1:3.5, 7:1:7, 7:1:10, 7:1:15, 7:1:17.5, or 7:1:20. In certain preferred embodiments, (+):P:(−) is about 10:1:10, 10:1:15, 10:1:20, 10:1:25, 10:1:30, or 10:1:40.

One of skill in the art will appreciate that amino-functionalized chitosan polyplex particles in complex with the anionic portion-containing polymer can be characterized by a three-component ratio of amino functional groups of the (e.g., dually) derivatized-chitosan polyplex (N) to phosphorus atoms of the nucleic acid (P) to anion moieties of the polymer (A), referred to as the “N:P:A molar ratio”. In certain embodiments, where N:P is from at least 2:1 to no more than 20:1, the molar ratio of P:A can vary from at least 1:40 to about 40:1.

In certain embodiments, where N:P is from at least 2:1 to no more than 20:1, the molar ratio of P:A can vary from at least 1:40 to about 1:10. In certain embodiments, where N:P is from at least 2:1 to no more than 20:1, the molar ratio of P:A can vary from at least 1:25 to about 25:1. In certain embodiments, where N:P is from at least 2:1 to no more than 20:1, the molar ratio of P:A can vary from at least 1:25 to about 1:10. In some cases, where N:P is from at least 2:1 to no more than 20:1, the molar ratio of P:A can vary from at least 1:20 to about 20:1. In some cases, where N:P is from at least 2:1 to no more than 20:1, the molar ratio of P:A can vary from at least 1:20 to about 1:10. In some cases, where N:P is from at least 2:1 to no more than 20:1, the molar ratio of P:A can vary from at least 1:10 to about 10:1. In some cases, where N:P is from at least 2:1 to no more than 20:1, the molar ratio of P:A can vary from at least 1:25 to about 2:1. In some cases, where N:P is from at least 2:1 to no more than 20:1, the molar ratio of P:A can vary from at least 1:20 to about 1:1.

In certain preferred embodiments, N:P:A is from 3:1:3.5 to 3:1:17.5. In certain preferred embodiments, N:P:A is from 5:1:3.5 to 5:1:17.5. In certain preferred embodiments, N:P:A is from 7:1:3.5 to 7:1:17.5. In certain preferred embodiments, N:P:A is from 10:1:10 to 10:1:40. In certain preferred embodiments, N:P:A is about 3:1:3.5, 3:1:7, 3:1:10, 3:1:15, 3:1:17.5, or 3:1:20. In certain preferred embodiments, N:P:A is about 5:1:3.5, 5:1:7, 5:1:10, 5:1:15, 5:1:17.5, or 5:1:20. In certain preferred embodiments, N:P:A is about 7:1:3.5, 7:1:7, 7:1:10, 7:1:15, 7:1:17.5, or 7:1:20. In certain embodiment, N:P:A is about 10:1:10, 10:1:15, 10:1:20, 10:1:25, 10:1:30 or 10:1:40.

1.4.1. Hydrophilic Non-Charged Portion

The hydrophilic non-charged portion of the polymer can be, or comprise, a polyalkylene polyol or a polyalkyleneoxy polyol portion, or combinations thereof. The hydrophilic non-charged portion of the polymer can be, or comprise, a polyalkylene glycol or polyalkyleneoxy glycol portion. In certain embodiments, the polyalkylene glycol portion is or comprises a polyethylene glycol portion and/or a monomethoxy polyethylene glycol portion. In certain preferred embodiments, the non-charged portion of the polymer is, or comprises polyethylene glycol. The hydrophilic non-charged portion of the polymer can be, or comprise, other biologically compatible polymer(s) such as polylactic acid.

In addition to PEG, several hydrophilic non-charged entities are known in the art. For example, see: Lowe et. al., Antibiofouling polymer interfaces: poly(ethyleneglycol) and other promising candidates, Polym. Chem., 6, 198-212, 2015, and Knop et. al., Poly(ethylene glycol) in Drug Delivery: Pros and Cons as Well as Potential Alternatives. Angewandte Chemie International Edition, 49(36), 6288-6308, 2010. Examples of hydrophilic non-charged portion of the polymer are but not limited to: poly(glycerol), poly(2-methacryloyloxyethyl phosphorylcholine), poly(sulfobetaine methacrylate), and poly(carboxybetaine methacrylate), poly(2-methyl-2-oxazoline), poly(2-ethyl-2-oxazoline), and poly(vinylpyrrolidone).

The hydrophilic portion can have a weight average molecular weight of from about 500 Da to about 50,000 Da. In some embodiments, the hydrophilic portion has a weight average molecular weight of from about 1,000 Da to about 10,000 Da. In certain embodiments, the hydrophilic portion has a weight average molecular weight of from about 1,500 Da to about 7,500 Da. In certain embodiments, the hydrophilic portion has a weight average molecular weight of from about 3,000 Da to about 5,000 Da. In some cases, the hydrophilic portion has a weight average molecular weight of, or of about, 5,000 Da.

1.4.2. Anionic Polymer Portion

The anionic polymer portion of the polymer can comprise a plurality of functional groups that are negatively charged at physiological pH. A wide variety of anionic polymers are suitable for use in the methods and compositions described herein, provided that such anionic polymers can be provided as a component of a polymer having a hydrophilic non-charged polymer portion and are capable of forming a (e.g., reversible) charge:charge complex with the positively charged (e.g., dually) derivatized-chitosan-nucleic acid nanoparticles.

Exemplary anionic polymers include, but are not limited to, polypeptides having a net negative charge at physiological pH. In some cases, the polypeptides, or a portion thereof, consist of amino acids having a negatively charged side-chain at physiological pH. For example, the anionic polymer portion of the polymer can be a polyglutamate polypeptide, a polyaspartate polypeptide, or a mixture thereof. Additional amino acids, or mimetics thereof, can be incorporated into the polyanionic polypeptide. For example, glycine and/or serine amino acids can be incorporated to increase flexibility or reduce secondary structure.

In some cases, the anionic polymers can be or comprise an anionic carbohydrate polymer. Exemplary anionic carbohydrate polymers include, but are not limited to, glycosaminoglycans that are negatively charged at physiological pH. Exemplary anionic glycosaminoglycans include, but are not limited to, chondroitin sulfate, dermatan sulfate, keratin sulfate, heparin, heparin sulfate, hyaluronic acid, or a combination thereof. In certain embodiments, the anionic polymer portion of the polymer is or comprises hyaluronic acid.

Additional or alternative anionic carbohydrate polymers can include polymers comprising dextran sulfate.

In some cases, the polyanion portion is, or comprises, a polyanion selected from the group consisting of polymethacrylic acid and its salts, polyacrylic acid and its salts, copolymers of methacrylic acids and its salts, and copolymers of acrylic acid and/or methacrylic acid and its salts, such as a polyalkylene oxide, polyacrylic acid copolymer.

In some cases, the polyanion portion is, or comprises, a polyanion is selected from the group consisting of alginate, carrageenan, furcellaran, pectin, xanthan, hyaluronic acid, heparin, heparan sulfate, chondroitin sulfate, cellulose, oxidized cellulose, carboxymethyl cellulose, croscarmellose, synthetic polymers and copolymers containing pendant carboxyl groups, phosphate groups or sulphate groups, polyaminoacids of predominantly negative charge, and biocompatible polyphenolic materials.

The anionic portion of the polymers can have a weight average molecular weight of from about 500 Da to about 5,000 Da. In some embodiments, the anionic portion has a weight average molecular weight of from about 500 Da to about 3,000 Da. In certain embodiments, the anionic portion has a weight average molecular weight of from about 500 Da to about 2,500 Da. In certain embodiments, the anionic portion has a weight average molecular weight of from about 500 Da to about 2,000 Da. In certain embodiments, the anionic portion has a weight average molecular weight of from about 500 Da to about 1,500 Da. In some embodiments, the anionic portion has a weight average molecular weight of from about 1,000 Da to about 5,000 Da. In some embodiments, the anionic portion has a weight average molecular weight of from about 1,000 Da to about 3,000 Da. In certain embodiments, the anionic portion has a weight average molecular weight of from about 1,000 Da to about 2,500 Da. In certain embodiments, the anionic portion has a weight average molecular weight of from about 1,000 Da to about 2,000 Da. In some cases, the anionic portion has a weight average molecular weight of, or of about, 1,500 Da.

As used herein, “block copolymer”, “block co-polymer”, and the like refers to a copolymer containing distinct homopolymer regions. A diblock copolymer contains two distinct homopolymer regions. A triblock copolymer contains three distinct homopolymer regions. The three distinct regions can each be different (e.g., AAAA-BBBB-CCCC), or two regions can be the same (e.g., AAAA-BBBB-AAAA) similar (e.g., AAAA-BBBB-AAA), wherein “A”, “B”, and “C” represent different monomer subunits that form copolymer is comprised. For example, “A” can represent an ethylene glycol monomer subunit of a polyethylene glycol homopolymer and B can represent a glutamic acid subunit of a polyglutamic acid homopolymer. The block copolymer can be a linear (e.g., di- or tri-) block copolymer. Exemplary embodiments of linear diblock and triblock copolymers for use in the subject invention include those listed in the following non-exhaustive list:

PEG-Polyglutamic acid
methoxy-poly(ethylene glycol)-block-poly(L-glutamic acid)
mPEG*K-b-PLE##
mPEG1K-b-PLE10
mPEG1K-b-PLE50
mPEG1K-b-PLE100
mPEG1K-b-PLE200
mPEG5K-b-PLE10
mPEG5K-b-PLE50
mPEG5K-b-PLE100
mPEG5K-b-PLE200
mPEG10K-b-PLE10
mPEG10K-b-PLE50
mPEG10K-b-PLE100
mPEG10K-b-PLE200
mPEG20K-b-PLE10
mPEG20K-b-PLE50
mPEG20K-b-PLE100
mPEG20K-b-PLE200
PEG-Polyaspartic acid
methoxy-poly(ethylene glycol)-block-poly(L-aspartic acid)
mPEG*K-b-PLD##
mPEG1K-b-PLD10
mPEG1K-b-PLD50
mPEG1K-b-PLD100
mPEG1K-b-PLD200
mPEG5K-b-PLD10
mPEG5K-b-PLD50
mPEG5K-b-PLD100
mPEG5K-b-PLD200
mPEG20K-b-PLD10
mPEG20K-b-PLD50
mPEG20K-b-PLD100
mPEG20K-b-PLD200
PGA-PEG-PGA
poly(L-glutamic acid)-block-poly(ethylene glycol)-block-
poly(L-glutamic acid)
PLE##-b-PEG*K-b-PLE##
PLE10-b-PEG1K-b-PLE10
PLE50-b-PEG1K-b-PLE50
PLE100-b-PEG1K-b-PLE100
PLE10-b-PEG5K-b-PLE10
PLE50-b-PEG5K-b-PLE50
PLE100-b-PEG5K-b-PLE100
Polyaspartic-PEG-polyaspartic
poly(L-aspartic acid)-block-poly(ethylene glycol)-block-poly(L-
aspartic acid)
PLD##-b-PEG*K-b-PLD##
PLD10-b-PEG1K-b-PLD10
PLD50-b-PEG1K-b-PLD50
PLD100-b-PEG1K-b-PLD100
PLD10-b-PEG5K-b-PLD10
PLD50-b-PEG5K-b-PLD50
PLD100-b-PEG5K-b-PLD100
PEG-poly glutamic acid-PEG
Methoxy-poly(ethylene glycol)-block-poly(L-glutamic acid)-block-
poly(ethylene glycol)
PEG*K-b-PGA##-b-PEG*K
PEG1K-b-PGA10-b-PEG1K
PEG1K-b-PGA50-b-PEG1K
PEG1K-b-PGA100-b-PEG1K
PEG5K-b-PGA10-b-PEG5K
PEG5K-b-PGA50-b-PEG5K
PEG5K-b-PGA100-b-PEG5K
PEG-polyaspartic-PEG
Methoxy-poly(ethylene glycol)-block-poly(L-aspartic acid)-block-
poly(ethylene glycol)
PEG*K-b-PLD##-b-PEG*K
PEG1K-b-PLD10-b-PEG1K
PEG1K-b-PLD50-b-PEG1K
PEG1K-b-PLD100-b-PEG1K
PEG5K-b-PLD10-b-PEG5K
PEG5K-b-PLD50-b-PEG5K
PEG5K-b-PLD100-b-PEG5K
*K: molecular weight of PEG in kDa
## number of subunits

In one embodiment, the block copolymer is or comprises a PEG-polyglutamic acid polymer having the following structure:

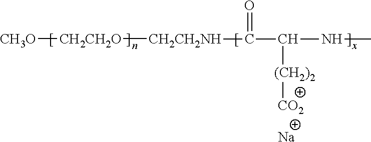

In one embodiment, the block copolymer is or comprises a PEG-polyaspartic acid polymer having the following structure:

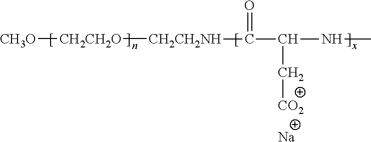

In one embodiment, the block copolymer is or comprises a PEG-hyaluronic acid polymer having the following structure:

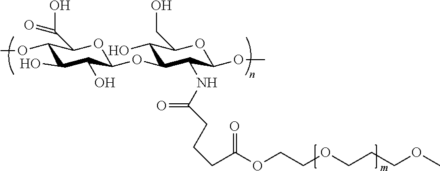

1.5. Alternative Cationic Polymers and Lipids

The nucleic acid polyplexes of the subject invention function to condense and protect the nucleotides from enzymatic degradation. In addition to chitosan and derivatives thereof, alternative materials that can also be advantageously used for this purpose include other positively-charged (i.e. cationic) polymers and/or lipids.

Examples of cationic polymers that can be used to form polyplexes with the therapeutic nucleic acid constructs of the current disclosure include polyamines; polyorganic amines (e.g., polyethyleneimine (PEI), polyethyleneimine celluloses, and derivatives thereof); poly(amidoamines) (PAMAM and derivatives thereof); polyamino acids (e.g., polylysine (PLL), polyarginine, and derivatives thereof); polysaccharides (e.g., cellulose, dextran, DEAE dextran, starch); spermine, spermidine, poly(vinylbenzyl trialkyl ammonium), poly(4-vinyl-N-alkyl-pyridiumiun), poly(acryloyl-trialkyl ammonium), and Tat proteins. See, e.g., Samal et al., Cationic polymers and their therapeutic potential, Chem Soc Rev. 41:7147-94 (2012)

Examples of positively-charged lipids include esters of phosphatidic acid with an aminoalcohol, such as an ester of dipalmitoyl phosphatidic acid or distearoyl phosphatidic acid with hydroxyethylenediamine. More particular examples of positively charged lipids include 3β-[N—(N′,N′-dimethylaminoethyl)carbamoyl) cholesterol (DC-chol); N,N′-dimethyl-N,N′-dioctyl ammonium bromide (DDAB); N,N′-dimethyl-N,N′-dioctacyl ammonium chloride (DDAC); 1,2-dioleoyloxypropyl-3-dimethyl-hydroxyethyl ammonium chloride (DORI); 1,2-dioleoyloxy-3-[trimethylammonio]-propane (DOTAP); N-(1-(2,3-dioleyloxy)propyI)-N,N,N-trimethylammonium chloride (DOTMA); dipalmitoylphosphatidylcholine (DPPC); 1,2-dioctadecyloxy-3-[trimethylammonio]-propane (DSTAP); and the cationic lipids described in e.g. Martin et al., Current Pharmaceutical Design 2005, 11, 375-394.

Blends of lipids and polymers in any concentration and in any ratio can also be used. Blending different polymer types in different ratios using various grades can result in characteristics that borrow from each of the contributing polymers. Various terminal group chemistries can also be adopted.

1.6. Methods of Making

As described above, one of skill in the art will appreciate that polyplex:polymer particles of the invention may be produced by a variety of methods. For example, polyplex particles can be generated and then contacted with polymer. In an exemplary non-limiting embodiment, polyplex particles are prepared by providing and combining functionalized chitosan and nucleotide feedstock. Feedstock concentrations may be adjusted to accommodate various amino-to-phosphate ratios (N/P), mixing ratios and target nucleotide concentrations. In some embodiments, particularly small batches, e.g., batches under 2 mL, the functionalized chitosan and nucleotide feedstocks may be mixed by slowly dripping the nucleotide feedstock into the functionalized chitosan feedstock while vortexing the container. In other embodiments, the functionalized chitosan and nucleotide feedstocks may be mixed by in-line mixing the two fluid streams. In other embodiments, the resulting polyplex dispersion may be concentrated by means known in the art such as ultrafiltration (e.g., tangential flow filtration (TFF)), or solvent evaporation (e.g., lyophilization or spray drying). A preferred method for polyplex formation is disclosed in WO 2009/039657, which is expressly incorporated herein in its entirety by reference.

Similarly, polyplex particle feedstock (e.g., an aqueous solution comprising the polyplex compositions) can be provided (e.g., isolated from the reaction mixtures described above) and combined with polymer feedstock (e.g., an aqueous solution comprising the polymer). Feedstock concentrations may be adjusted to accommodate various amino-to-anion ratios (N/A), amino-to-phosphorous (N:P) ratios, N:P:A ratios, mixing ratios and target nucleotide concentrations. In some embodiments, particularly small batches, e.g., batches under 2 mL, the feedstocks may be mixed by slowly dripping a first feedstock (e.g., polyplex) into a second feedstock (e.g., polymer) while vortexing the container. In other embodiments, the feedstocks may be mixed by in-line mixing the two fluid streams. In other embodiments, the resulting polyplex:polymer complex dispersion may be concentrated by means known in the art such as ultrafiltration (e.g., tangential flow filtration (TFF)), or solvent evaporation (e.g., lyophilization or spray drying).

2. Powdered Formulations

The polyplex:polymer compositions of the invention include powders. In a preferred embodiment, the invention provides a dry powder polyplex:polymer composition. In a preferred embodiment, the dry powder polyplex:polymer composition is produced through the dehydration (e.g., spray drying or lyophilization) of a chitosan-nucleic acid polyplex dispersion of the invention.

3. Pharmaceutical Formulations

The present invention also provides “pharmaceutically acceptable” or “physiologically acceptable” formulations comprising polyplex:polymer compositions of the invention. Such formulations can be administered in vivo to a subject in order to practice treatment methods.

As used herein, the terms “pharmaceutically acceptable” and “physiologically acceptable” refer to carriers, diluents, excipients and the like that can be administered to a subject, preferably without producing excessive adverse side-effects (e.g., nausea, abdominal pain, headaches, etc.). Such preparations for administration include sterile aqueous or non-aqueous solutions, suspensions, and emulsions. Liquid formulations include suspensions, solutions, syrups and elixirs. Liquid formulations may be prepared by the reconstitution of a solid.

Pharmaceutical formulations can be made from carriers, diluents, excipients, solvents, dispersion media, coatings, antibacterial and antifungal agents, isotonic and absorption delaying agents, and the like, compatible with administration to a subject. Such formulations can be contained in a tablet (coated or uncoated), capsule (hard or soft), microbead, emulsion, powder, granule, crystal, suspension, syrup or elixir. Supplementary active compounds and preservatives, among other additives, may also be present, for example, antimicrobials, anti-oxidants, chelating agents, and inert gases and the like.

Excipients can include a salt, an isotonic agent, a serum protein, a buffer or other pH-controlling agent, an anti-oxidant, a thickener, an uncharged polymer, a preservative or a cryoprotectant. Excipients used in compositions of the invention may further include an isotonic agent and a buffer or other pH-controlling agent. These excipients may be added for the attainment of preferred ranges of pH (about 6.0-8.0) and osmolarity (about 50-400 mmol/L). Examples of suitable buffers are acetate, borate, carbonate, citrate, phosphate and sulfonated organic molecule buffer. Such buffers may be present in a composition in concentrations from 0.01 to 1.0% (w/v). An isotonic agent may be selected from any of those known in the art, e.g. mannitol, dextrose, glucose and sodium chloride, or other electrolytes. Preferably, the isotonic agent is glucose or sodium chloride. The isotonic agents may be used in amounts that impart to the composition the same or a similar osmotic pressure as that of the biological environment into which it is introduced. The concentration of isotonic agent in the composition will depend upon the nature of the particular isotonic agent used and may range from about 0.1 to 10%. When glucose is used, it is preferably used in a concentration of from 1 to 5% w/v, more particularly 5% w/v. When the isotonic agent is sodium chloride, it is preferably employed in amounts of up to 1% w/v, in particular 0.9% w/v. The compositions of the invention may further contain a preservative. Examples preservatives are polyhexamethylene-biguanidine, benzalkonium chloride, stabilized oxychloro complexes (such as those known as Purite®), phenylmercuric acetate, chlorobutanol, sorbic acid, chlorhexidine, benzyl alcohol, parabens, and thimerosal. Typically, such preservatives are present at concentrations from about 0.001 to 1.0%. Furthermore, the compositions of the invention may also contain a cryopreservative agent. Preferred cryopreservatives are glucose, sucrose, mannitol, lactose, trehalose, sorbitol, colloidal silicon dioxide, dextran of molecular weight preferable below 100,000 g/mol, glycerol, and polyethylene glycols of molecular weights below 100,000 g/mol or mixtures thereof. Most preferred are glucose, trehalose and polyethylene glycol. Typically, such cryopreservatives are present at concentrations from about 0.01 to 10%.

A pharmaceutical formulation can be formulated to be compatible with its intended route of administration. For example, for oral administration, a composition can be incorporated with excipients and used in the form of tablets, troches, capsules, e.g., gelatin capsules, or coatings, e.g., enteric coatings (Eudragit® or Sureteric®). Pharmaceutically compatible binding agents, and/or adjuvant materials can be included in oral formulations. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or other stearates; a glidant such as colloidal silicon dioxide; a sweetening agent such as sucrose or saccharin; or a flavoring agent such as peppermint, methyl salicylate, or flavoring.

Formulations can also include carriers to protect the composition against rapid degradation or elimination from the body, such as a controlled release formulation, including implants and microencapsulated delivery systems. For example, a time delay material such as glyceryl monostearate or glyceryl stearate alone, or in combination with a wax, may be employed.

Suppositories and other rectally administrable formulations (e.g., those administrable by enema) are also contemplated. Further regarding rectal delivery, see, for example, Song et al., Mucosal drug delivery: membranes, methodologies, and applications, Crit. Rev. Ther. Drug. Carrier Syst., 21:195-256, 2004; Wearley, Recent progress in protein and peptide delivery by noninvasive routes, Crit. Rev. Ther. Drug. Carrier Syst., 8:331-394, 1991.

Additional pharmaceutical formulations appropriate for administration are known in the art and are applicable in the methods and compositions of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Techonomic Publishing Co., Inc., Lancaster, Pa., (1993)).

4. Administration

In one embodiment, the use of polyplexes:polymer compositions provides for prolonged stability of polyplexes at physiological pH. This provides for effective mucosal administration.

Any of a number of administration routes to contact mucosal cells or tissue are possible and the choice of a particular route will in part depend on the target mucosal cell or tissue. Syringes, endoscopes, cannulas, intubation tubes, catheters, nebulizers, inhalers and other articles may be used for administration.

Intravesical administration of chemotherapeutic agents is standard care for some bladder cancers. Briefly, intravesical therapy involves instillation of a therapeutic agent directly into the bladder via insertion of a urethral catheter. In some embodiments, the subject compositions provide for enhanced stability in urine, thereby improving localized expression.

The doses or “effective amount” for treating a subject are preferably sufficient to ameliorate one, several or all of the symptoms of the condition, to a measurable or detectable extent, although preventing or inhibiting a progression or worsening of the disorder or condition, or a symptom, is a satisfactory outcome. Thus, in the case of a condition or disorder treatable by expressing a therapeutic nucleic acid in target tissue, the amount of therapeutic RNA or therapeutic protein produced to ameliorate a condition treatable by a method of the invention will depend on the condition and the desired outcome and can be readily ascertained by the skilled artisan. Appropriate amounts will depend upon the condition treated, the therapeutic effect desired, as well as the individual subject (e.g., the bioavailability within the subject, gender, age, etc.). The effective amount can be ascertained by measuring relevant physiological effects.

Veterinary applications are also contemplated by the present invention. Accordingly, in one embodiment, the invention provides methods of treating non-human mammals, which involve administering a polyplex:polymer composition of the invention to a non-human mammal in need of treatment. The compositions of the invention may also be administered to the mucosa. For example, the compositions can be administered to mucosal cells or tissue of the gastrointestinal tract, including but not limited to mucosal cells or tissues of the small intestine and/or large intestine. Other target mucosal cells or tissues include, but are not limited to ocular, airway epithelial, lung, vaginal, and bladder cells or tissues.

Typical formulations for this purpose include liquids, gels, hydrogels, solutions, creams, foams, films, implants, sponges, fibers, powders, and microemulsions.

The compounds of the invention can be administered to the mucosa intranasally or by inhalation, typically in the form of a dry powder (either alone, as a mixture, for example, in a dry blend with lactose, or as a mixed component particle) from a dry powder inhaler or as an aerosol spray from a pressurized container, pump, spray, atomizer, or nebulizer, with or without the use of a suitable propellant.

Capsules, blisters and cartridges for use in an inhaler or insufflator may be formulated to contain a powder mix of the compound of the invention, a suitable powder base such as lactose or starch and a performance modifier such as I-leucine, mannitol, or magnesium stearate.

Formulations for inhaled/intranasal administration may be formulated to be immediate and/or modified release. Modified release formulations include delayed-, sustained-, pulsed-, controlled-, targeted and programmed release.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops. Other formulations suitable for ocular and aural administration include ointments, biodegradable (e.g. absorbable gel sponges, collagen) and non-biodegradable (e.g. silicone) implants, wafers, lenses and particulate systems. Formulations may also be delivered by iontophoresis.

Formulations for ocular/aural administration may be formulated to be immediate and/or modified release. Modified release formulations include delayed, sustained, pulsed, controlled, targeted, or programmed release.

Mucosal Administration

In preferred embodiments, the compositions of the invention are administered to the mucosa. For example, the compositions can be administered to mucosal cells or tissue of the bladder and gastroinstinal tract, including but not limited to mucosal cells or tissues of the small intestine and/or large intestine and/or colon. Other target mucosal cells or tissues include, but are not limited to ocular, airway epithelial, lung, vaginal, and bladder cells or tissues.

Typical formulations for this purpose include liquids, gels, hydrogels, solutions, creams, foams, films, implants, sponges, fibres, powders, and microemulsions.

In an exemplary embodiment for the bladder mucosa, the compounds described herein can be administered using intravesical therapy. Intravesical therapy involves instillation of a therapeutic agent directly into the bladder via insertion of a urethral catheter. The agent is allowed to sit in the bladder for a period of time, between 0.5 and 6 hours. It is a standard route of administration for bladder cancer chemotherapies. It utilizes the outside anatomical access available for drug delivery directly to the disease site in bladder and thereby avoids unwanted exposure of the instilled drug to healthy tissues elsewhere in the body.

Formulations for bladder administration may be formulated to be immediate and/or modified release. Modified release formulations include delayed-, sustained-, pulsed-, controlled-, targeted, or programmed release

The compounds of the invention can also be administered to the mucosa intranasally or by inhalation, typically in the form of a dry powder (either alone, as a mixture, for example, in a dry blend with lactose, or as a mixed component particle) from a dry powder inhaler or as an aerosol spray from a pressurized container, pump, spray, atomiser, or nebuliser, with or without the use of a suitable propellant.

Capsules, blisters and cartridges for use in an inhaler or insufflator may be formulated to contain a powder mix of the compound of the invention, a suitable powder base such as lactose or starch and a performance modifier such as I-leucine, mannitol, or magnesium stearate.

Formulations for inhaled/intranasal administration may be formulated to be immediate and/or modified release. Modified release formulations include delayed-, sustained-, pulsed-, controlled-, targeted and programmed release.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops. Other formulations suitable for ocular and aural administration include ointments, biodegradable (e.g. absorbable gel sponges, collagen) and non-biodegradable (e.g. silicone) implants, wafers, lenses and particulate systems. Formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Therapeutic Applications

Therapeutic proteins contemplated for use in the invention have a wide variety of activities and find use in the treatment of a wide variety of disorders. The following description of therapeutic protein activities, and indications treatable with therapeutic proteins of the invention, is exemplary and not intended to be exhaustive. The term “subject” refers to an animal, with mammals being preferred, and humans being especially preferred. Specific non-limiting examples of therapeutic embodiments are described below. In some cases, the therapeutic embodiments are intended to act on non-mucosal target tissues, cells, or organs. Where the therapeutic effect is non-mucosal, it is understood that the cells or tissues contacted by the polyplex:polymer compositions described herein are mucosal and the therapeutic action is proximal to the mucosal target. For example, mucosal cells can be transfected to produce and secrete IL-12 and/or another immunostimulatory molecule.

In one embodiment, polyplex:polymer compositions of the invention may be used for therapeutic treatment. Such compositions are sometimes referred to herein as therapeutic compositions. As noted above, the subject compositions and methods primarily employ therapeutic nucleic acids encoding IL-12, either alone or in conjunction with additional innate and/or adaptive immunostimulatory molecules. In some embodiments, the therapeutic nucleic acid further encodes an IFN-1 activator/inducer such as, e.g., a RIG-I agonist, a STING agonist, a TLR 7/9 agonist, and/or other Pattern Recognition Receptor agonists. See, e.g. Vasou et al., Viruses 9:186 (2017). In some embodiments, the therapeutic nucleic acid further encodes a modulator of an immune checkpoint molecule selected from the group consisting of CTLA-4, PD-1, PD-L1, PD-L2, TIM43, B7-H₃, B7-H₄, LAG-3, KIR, and ligands thereof.

Suitable IFN-1 activator/inducers include RIG-I agonists (such as eRNA11a, adenovirus VA RNA1, eRNA41H, MK4621 (Merck), SLR10, SLR14, and SLR20), STING (i.e., stimulator of interferon genes) agonists (such as CDN, i.e., cyclic dinucleotides), PRRago (such as CpG, Imiquimod, or Poly I:C), and TLR agonists (such as CPG-1826, GS-9620, AED-1419, CYT-003-QbG10, AVE-0675, or PF-7909) including IRL7 and TLR9, and RLR stimulators (such as RIG-I, Mda5, or LGP2 stimulators). In some embodiments, the IFN-1 activator/inducer induces dendritic cells, T cells, B cells, and/or T follicular helper cells.

In preferred embodiments, the IFN-1 activator/inducer is a RIG-I agonist. RIG-I (retinoic acid inducible gene I, encoded by Ddx58) is a cytosolic antiviral helicase that acts as an RNA sensor, detecting and being activated upon recognition of viral RNAs in the cytoplasm. A pattern recognition receptor, RIG-I contains an RNA helicase domain and two N-terminal caspase recruitment domains (CARDs), which relay a signal to the downstream signaling adaptor MAVS (mitochondrial antiviral-signaling protein). RIG-1 signaling via MAVS leads to a variety of responses including induction of type I IFN responses, including IFNα and IFNβ via TBK1 and IRF7/8, and activation of caspase-8-dependent apoptosis. They are found in most tissues, including cancer cells (Kato et al., Immunol. Rev. 243(1):91-98 (2011)).

RIG-I induced responses differs between cells. While normal healthy cells such as melanocytes and fibroblasts are quite resistant to RIG-I-induced apoptosis, tumor cells are highly susceptible to RIG-I-induced cell death (Besch et al., 2009; Kubler et al., 2010). RIG-I's natural ligands are viral short blunt ends of duplex RNA containing 5′ tri or diphosphate (5′ ppp or 5′ pp). RIG-I-specific ligands are currently being developed for immunotherapy of cancer (Duewell et al., 2014, 2015; Ellermeier et al., 2013; Schnurr & Duewell, Oncoimmunology, 2(5):e24170 (2013) and, 2014). Part of the potent antitumor activity of RIG-I ligands is the downstream ability to promote cross-presentation of antigens to CD8 T cells and to induce cytotoxic activity (Hochheiser et al., 2016). RIG-I ligands also show strong therapeutic activity in viral infection models such as influenza (Weber-Gerlach & Weber, 2016).

Plasmid vector backbones expressing RIG-I ligands from RNA polymerase III promoters have been used to identify potent synthetic RIG-I ligands (Luke et al., J. Virol. 85(3):1370-1383). Stemloop RNA modified with tri-phosphate are of particular use as agonists in the instant invention. These include, but are not limited to, eRNA41H, which combines (i) eRNA11a, an immunostimulatory dsRNA expressed by convergent transcription, with (ii) adenovirus VA RNAI, SLR20, a double-stranded, triphosphorylated 20-base pair stem-loop RNA, modified with a 5′ triphosphate sequence (Elion et al., Cancer Res. 78(21):6183-6195 (2018)), and SLR10 and SLR14, which are alternative polyphosphorylated RNAs with a stable tetraloop at one end (Jiang et al., J. Exp. Med. 216:2854-68 (2019)).

Additional RIG-I agonists finding advantageous use in the compositions and methods described herein include SB-9200, a broad-spectrum antiviral innate sensor agonist that acts via the activation of the RIG-I and nucleotide-binding oligomerization domain 2 pathway (Jones et al. J. Med. Virol. 89:1620-1628 (2017), MK 4621 (RGT100, Merck), CBS-13-BPS, a synthetic RIG-I-specific agonist mimicking the structure of the influenza virus panhandle promoter (Lee et al. Nucleic Acids Res. 46:10553 (2018); IVT-B2 RNA (Lien et al. Molecular Therapy 24:135-45 (2016), SeV DVGs (Xu et al., mBio 65:e01265-15 (2015)), 5′ ppp RNA with uridine-rich sequence with 99 nucleotides hairpin (M8) (Chiang et al. J. Virol. 89:8011-25 (2015), and 3 pRNA.

In accordance with the foregoing embodiments, RIG-I agonists suitable for co-expression with IL-12 in the subject compositions and methods include, but are not limited to: RIG-I DNA vaccines, plasmid encoded RNA polymerase III expressed RNA-based RIG-I agonists such as, e.g., eRNA11a, adenovirus VA RNA1, eRNA41H (Nature Technology Corp), GFP2, Lamin A/C and Lamin VSV, tri-GFPs, SAD ΔPLp, Tri-G-AC-U, Flu vRNA, RNaseL fragments, pppRVL, pppVSVL, ppp-shRNA-luc3VA1, 5′ppp-dsRNA, 3p-hpRNA, MK4621 (Merck), SLR10, SLR14, SLR20, CBS-13-BPS, IVT-B2 RNA, SeV CVG, SB-9200, and siRNAs as disclosed in Ellermeier et al., Cancer Research (2013) 73(6). Similarly, STING agonists suitable for coexpression with IL-12 include, but are not limited to: DExD/H helicases including DDX41, and TLR agonists include, but are limited to CpG dinucleotides such as, e.g., CpG-1826 (ODN1826, Invivogen).

In accordance with the foregoing embodiments, modulators of immune checkpoint molecules suitable for coexpression with IL-12 in the subject compositions and methods include, e.g., single domain antibodies (sdAb) directed to one or more of CTLA-4, PD-1, PD-L1, PD-L2, TIM3, B7-H₃, B7-H₄, LAG-3, and KIR (such as, e.g., KN035 (Ablynx/Sanofi); Inhibrix 105), (see also Wan et al., Oncol. Rep. (2018); Hosseinzadeh et al., Rep. Biochem & Mol. Bio., (2017); Dougan et al., Can. Imm. Res. (2016); Ingram et al., PNAS (2018), and WO2017198212); dominant negative PD-1 molecules (e.g., Atara Therapeutics), PD-1 variants having high affinity for PD-L1 (e.g. competitive antagonists) (Maute, PNAS (2015)); and CD80 variant(s) with increased binding to CD28 (e.g. WO2017/181152).

In some cases, said IFN-1 agonist and/or said immune checkpoint inhibitor is encoded by:
said therapeutic nucleic acid construct in said derivatized chitosan nucleic acid polyplex;
a different therapeutic nucleic acid construct in said derivatized chitosan nucleic acid polyplex;
a therapeutic nucleic acid construct in a different derivatized chitosan nucleic acid polyplex (e.g., that does not comprise a construct encoding IL-12);
a therapeutic nucleic acid construct (e.g., formulated in an alternate nucleic acid delivery formulation, such as a PEI or cationic lipid formulation).

The therapeutic nucleic acid construct encoding IL-12 and the therapeutic nucleic acid construct encoding said IFN-1 agonist and/or said immune checkpoint inhibitor can be simultaneously or sequentially administered. In some cases, the therapeutic nucleic acid construct encoding said IFN-1 agonist and/or said immune checkpoint inhibitor are co-administered in a single formulation or in single, e.g., admixed, combination of two different formulations. In some cases, the therapeutic nucleic acid construct encoding IL-12 and the therapeutic nucleic acid construct encoding said IFN-1 agonist and/or said immune checkpoint inhibitor are administered sequentially.

The immunostimulatory molecule of the invention may also encode an shRNA (short hairpin RNA) molecule designed to inhibit protein(s) involved in the growth or maintenance of tumor cells or other hyperproliferative cells. A plasmid DNA may simultaneously encode for a therapeutic protein and one or more shRNA. Furthermore, the nucleic acid of the said composition may also be a mixture of plasmid DNA and synthetic RNA including sense RNA, antisense RNA or ribozymes.

Methods of Treatment

### Hyperproliferative Disorders

The subject compositions and methods find advantageous use in the treatment of hyperproliferative disorders. Of particular interest are compositions and methods for the treatment of hyperproliferative disorders of mucosal tissues or in tissues proximal to mucosal tissue. Methods and compositions of the invention may be used in the treatment of gastrointestinal cancers including, but not limited to oral cancers, esophageal cancers, stomach cancers, pancreatic cancers, liver cancers, colorectal cancers, and rectal cancers. Nasal and pulmonary cancers which may be treated by the methods and compositions of the invention include, but are not limited to, paranasal sinus cancer, oropharyngeal cancer, tracheal cancer, and lung cancers. Genitourinary cancers which may be treated by the methods and compositions of the invention include, but are not limited to bladder cancers, urothelial cancers, urethral cancers, testicular cancers, kidney cancers, prostate cancers, penile cancers, adrenal cancers, uterine cancers, cervical cancers and ovarian cancers.

In some embodiments according to any one of the methods provided above, the method further comprises administering (such as systemically or locally to the site of the tumor) a non-nucleic acid-based immunostimulatory molecule.

In some embodiments, the immunostimulatory molecule is a modulator of an immune checkpoint molecule selected from the group consisting of CTLA-4, PD-1, PD-L1, PD-L2. TIM3, B7-H₃, B7-H₄, LAG-3, KIR, and ligands thereof. In some embodiments, the immunomodulator is an inhibitor of PD-L1 or PD-L1. In some embodiments, the inhibitor of PD-1 is an anti-PD-1 antibody, such as pembrolizumab or nivolumab. In some embodiments, the immunomodulator is an inhibitor of CTLA-4. In some embodiments, the inhibitor of CTLA-4 is an anti-CTLA-4 antibody, such as ipilimumab or tremelimumab. In some embodiments, the inhibitor of PD-L1 is an anti-PD-L1 antibody, such as atezolizumab.

In some embodiments, the immunomodulator is an IFN-1 agonist, e.g. a RIG-I agonist, a STING agonist, or a TLR 7/9 agonist. RIG-I agonists suitable for co-administration include, but are not limited to short poly I:C and polyAU compositions (e.g. Poly(I:C)/LyoVec complexes (Invivogen)); RGT100 (MK4621, Merck) SLR20 (Elion et al.; SLR10 & SLR14 (Jiang et al.); and agonists as disclosed in U.S. Pat. Nos. 8,871,799, 8,895,608, 8,927,561, 9,073,946, 9,458,492, 9,555,106, 9,884,876, 9,956,285, 9,775,894, 9,861,574, 9,937,247, U.S. Ser. No. 10/167,476, U.S. Ser. No. 10/350,158, U.S. Ser. No. 10/434,064, U.S. Ser. No. 10/273,484, U.S. Pat. Nos. 9,381,208B2, 9,738,680B2, 9,790,509, U.S. Ser. No. 10/059,943, U.S. Pat. Nos. 9,109,012B2, 9,937,247B2, 9,816,091B2, 9,133,456B2, 9,409,941B2, 9,340,789B2, 9,040,234B2, US20200071316, US20200063141A1, US20200061097A1, US20200055871A1, US20200016253A1, US20190076463A1, US20180195063A1, US20160287623A1.

STING agonists suitable for co-administration in conjunction with IL-12 include, but are not limited to c-Di-AMP sodium salt, c-Di-GMP sodium salt, 2′,3′-cGAMP sodium salt, 3′,3′-cGAMP sodium salt, 10-carboxymethyl-9-acridinone (CMA), DMXAA (Tocris Bioscience, InvivoGen, Nimbus Therapeutics), G10, α-Mangostin, CRD100 (Curadev), cAIMP, 2′ 2′-c-GAMP, 2′3′-cGAM(PS)2(Rp/Sp), 2′ 3′-c-di-AMP, c-di-IMP, c-di-UMP, 5,6-dimethylxanthenone-4-acetic acid (DMXAA), MK-1454 (Merck) ML RR-S2 CDG, ML RR-S2 CDA (ADU-S100), SB 11285 (Springbank Pharmaceuticals), MAVU (AbbVie), DiABZI, disodium dithio-(Rp1Rp)-[cyclic[A(2′ 5′)pA(3′ 5′)p]][Rp,Rp]-cyclic9 adenosine-(2′ 5′)-monophosphorothioate-adenosine-(3′ 5′)-monophosphorothioate), disodium (RR-S2 CDA, ADU-S100, MIW815)(Corrales et al., 2016) and the compositions disclosed in U.S. Pat. Nos. 10,176,292, 9,724,408, 10,011,630, 10,435,469, 10,414,747, 10,413,612, 10,131,686, 10,106,574, 10,047,115, 10,045,961, 10,011,630, 9,994,607, 9,937,247, 9,840,533, 9,770,467, 9,724,408, 9,718,848, and 9,642,830.

TLR7 and TLR9 agonists suitable for co-administration with IL-12 include, but are not limited to: imidazoquinolines and their analogs, including Resiquimod and Imiquimod (Aldara), hydroxychloroquine, chloroquine, bropirimine, Loxoribine, Isatoribine, CpG oligonucleotides, stabilized immune modulatory RNA (SIMRA) AST-008 (Exicure), MEDI9197 and the compositions disclosed in U.S. 434,064, U.S. Pat. Nos. 10,413,612, 10,407,431, 10,370,342, 10,364,266, 10,208,037, 10,202,386, 9,944,649, 9,902,730, 9,868,955, 9,359,360, 9,295,732, 9,243,050, 9,228,184, 9,216,192, 9,2206,430, 8,735,421, 8,728,486, 8,399,423 and 8,242,106.

In some embodiments, the non-nucleic acid-based immunomodulator and the subject compositions are administered simultaneously, such as in the same composition. In some embodiments, the non-nucleic acid-based immunomodulator and the subject compositions are administered sequentially.

In some embodiments, the methods for treating bladder cancer provided herein further comprise administering to the subject at least one additional therapeutic agent. In further embodiments, the additional therapeutic agent is a chemotherapeutic drug or a radiotherapeutic drug. In some embodiments, the chemotherapeutic drugs include, but are not limited to, cisplatin, carboplatin, paclitaxel, docetaxel, 5-fluorouracil 1, bleomycin, methotrexate, ifosfamide, oxaliplatin, cyclophosphamide, dacarbazine, temozolomide, gemcitabine, capecitabine, cladribine, clofarabine, cytarabine, floxuridine, fludarabine, hydroxyurea, pemetrexed, pentostatin, thioguanidine, daunorubicin, doxorubicin, epirubicin, idarubicin, topotecan, irinotecan, etoposide, eniposide, colchicine, vincristine, vinblastine, and vinorelbine. Exemplary cancer specific agents and antibodies include, but are not limited to, Afatinib, Aldesleukin, Alemtuzumab, Axitinib, Belimumab, Bevacizumab, Bortezomib, Bosutinib, Brentuximab vedotin, Cabozantinib, Canakinumab, Carfilzomib, Cetuximab, Crizotinib, Dabrafenib, Dasatinib, Denosumab, Erlotinib, Everolimus, Gefitinib, Ibritumomab tiuxetan, Ibrutinib, Imatinib, Ipilimumab, Lapatinib, Nilotinib, Obinutuzumab, Ofatumumab, Panitumumab, Pazopanib, Pertuzumab, Ponatinib, Regorafenib, Rituximab, Romidepsin, Ruxolitinib, Sipuleucel-T, Sorafenib, Temsirolimus, Tocilizumab, Tofacitinib, Tositumomab, Trametinib, Trastuzumab, Vandetanib, Vemurafenib, Vismodegib, Vorinostat, Ziv-aflibercept, and any combination thereof. In some embodiments, the additional therapeutic agent is administered to the subject prior to, concurrently with, or subsequent to administration of the immunoconjugate. In some embodiments, the additional therapeutic agent is administered systemically. For example, in some embodiments, the additional therapeutic agent is administered by intravenous injection.

Additionally, the conventional bladder cancer treatment currently approved in the U.S. is intra-urethral Bacillus Calmette-Guerin vaccine. This antigenic vaccine is thought to stimulate bladder cells to express interferon, which in turn recruits the patient's innate immune system to better recognize cancer cell surface antigens and attack cancer cells. In over a third of cases, however, the vaccine is ineffective. Similarly, intravesical instillation of exogenously manufactured interferon polypeptide has also been tested, but has not been effective. The subject compositions and methods can also be advantageously employed in conjunction with these more conventional approaches to augment and improve the immune response.

The examples set out herein illustrate several embodiments of the present disclosure but should not be construed as limiting the scope of the present disclosure in any manner.

### Example 1

Measure of Plasmid Transfection Efficacy In Vitro.

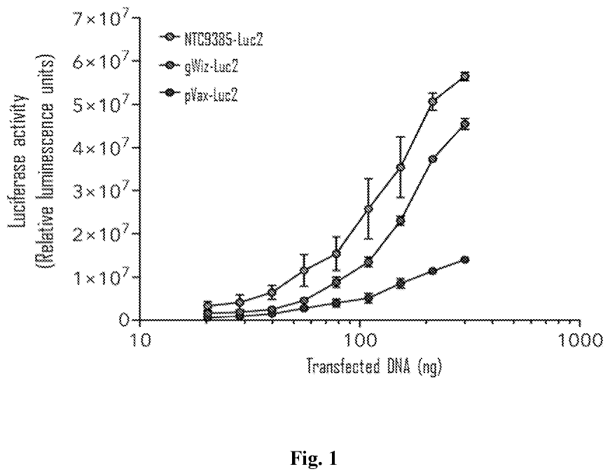

MB49 cells were seeded into 96-well plates (35,000 cells/well) prior to transfection with NTC9385-Luc2, gWiz-Luc2, or pVax-Luc2 plasmids. Transfection was performed using Lipofectamine2000 (Thermofisher) and increasing doses of plasmid DNA (20-300 ng). At 24 hours post-transfection, cells were lysed with Luciferase Cell Culture Lysis reagent (IX, Promega). Immediately after addition of Luciferin enzyme substrate to cell lysates bioluminescence was measured on a Envision plate reader (Perkin-Elmer). As seen in FIG. 1, NTC9358R-transfected cells showed highest efficiency in vitro.

### Example 2

Measure of Plasmid Transfection Efficacy In Vivo.

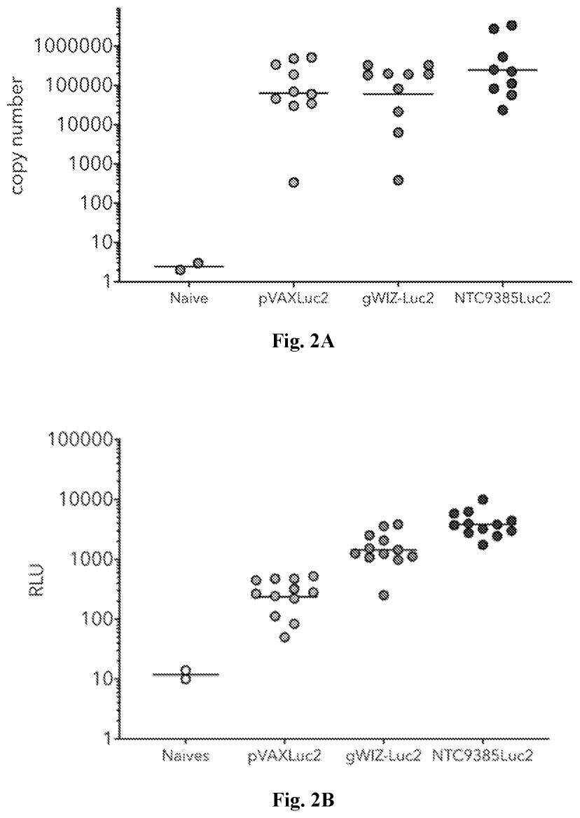

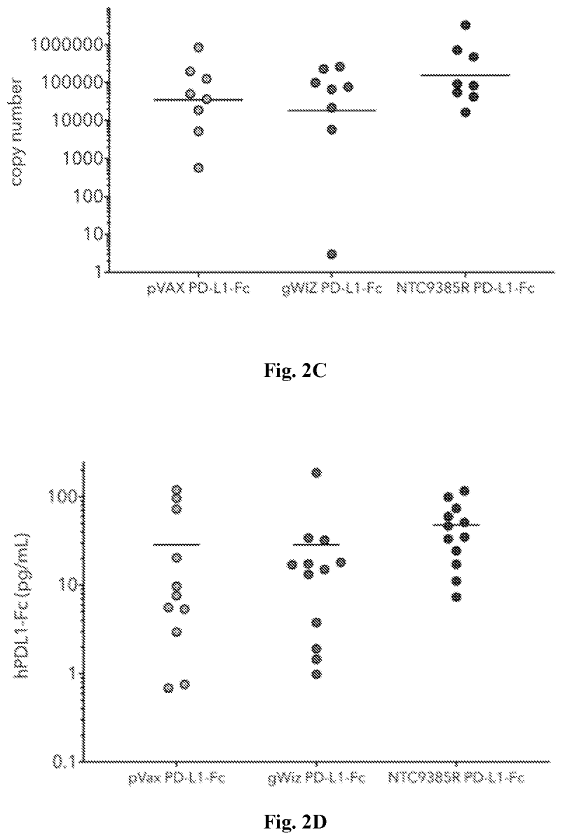

JetPEI-plasmid DNA polyplexes were prepared by mixing JetPEI and 20 μg of the candidate plasmids comprising optimized Luc2 at an amine-to-phosphate (NP) ratio of 6. Polyplexes were incubated at room temperature for a minimum of 15 minutes and used within 4 hours. Female mice (12-16 weeks) were administered 80 Id of Jet PEI-DNA formulation by intravesical instillation under anesthesia with isoflurane for 60 minutes of exposure time. At 24 hours post-administration, bladder tissue was harvested and assessed for transfection efficacy using mRNA expression and luciferase enzyme activity assays. RNA was extracted from harvested bladder tissue following homogenization in lysis buffer (Qiagen RNeasy). RT-qPCR was performed using 1 μg input RNA and TaqMan primer/probes that recognized Luc2. Absolute quantification was performed using a standard curve of Luc2 RNA. As seen in FIG. 2A, the RNA assay results were consistent with in vitro luciferase activity assay results, with the NTC9385 Luc2 showing highest mRNA levels post-transfection based on absolute copy number. For luciferase assays, harvested bladder tissue was lysed by homogenization in the presence of Luciferase Cell Culture Lysis Reagent (IX, Promega). The enzyme substrate, luciferin, was added to the tissue lysate and bioluminescence was measured immediately on the Envision plate reader (Perkin-Elmer). As seen in FIG. 2B, both the gWIZ-Luc2 and NTC9385 Luc2-plasmids showed significantly higher luciferase enzyme activity in vivo than the pVAX-Luc2 plasmid.

In order to assess efficacy of candidate plasmids to deliver a gene of interest in vivo, the transfections and post-transfection mRNA and protein expression assays were performed with plasmids bearing hPD-L1-Fc. For the RT-qPCR assay, at 24 h post-administration, bladder tissue was harvested, homogenized in lysis buffer and RNA was extracted (Qiagen RNeasy kit). RT-qPCR was performed using 1 μg input RNA and TaqMan primer/probes that recognize codon-optimized human PD-L1-Fc. Absolute quantification was performed using a standard curve of human PD-L1-Fc RNA. No statistically significant difference in hPD-L1-Fc mRNA expression was observed using the three different vectors at 24 hours-post administration (FIG. 2C). For hPD-L1-Fc protein expression, bladder tissue was harvested at 24 h post-administration and tissue was lysed by homogenization in lysis buffer with protease inhibitors. Human PD-L1-Fc protein was quantified using a custom-designed immunoassay (Mesoscale Discovery). Data is represented as pg/mL protein in lysate. As seen in FIG. 2D, NTC9385R demonstrates the least variable expression and only plasmid resulting in quantifiable hPDL1-Fc in all mice tested.

### Example 3

Promoter/Enhancer Screening in Mouse Bladder Epithelial Cell Line In Vitro

The efficacy of promoter/enhancer combinations were assessed by measuring dose-dependent expression of a green fluorescent reporter gene (GFP) following in vitro transfection of a murine urothelial cell line with a panel of plasmids containing various promoter/enhancer sequences. The promoter/enhancer sequences used in the assay were CAG, EF1a, CMV/EF1a/HTLV, CMV/UbC, EF1a/HTLV, 2×CMV/EF1a, CMV, UbC, CMV/EF1a, CMV/UbB, PGK, UbB, and CBA. MB49 cells (mouse bladder cells) were seeded into 96-well plates (35,000 cells/well) 24 hours prior to transfection with the indicated plasmids on the SnapFast backbone (pSF).

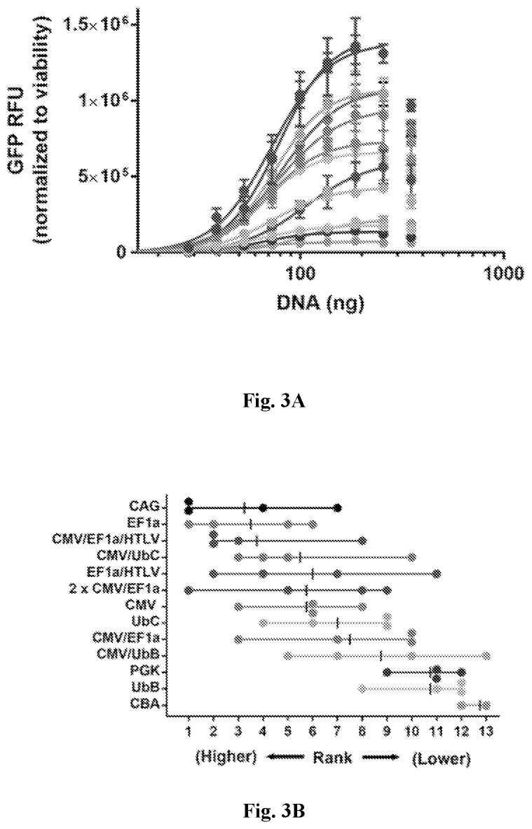

Transfection was performed using Lipofectamine2000 (ThermoFisher) and increasing doses of plasmid DNA. At 48 hours post-transfection, fluorescence was measured on the Envision plate reader. Cell viability was measured using the AlamarBlue Cell Viability Reagent (Invitrogen). Data is represented as GFP relative fluorescence units (RFU) normalized to cell viability (FIG. 3A). Total fluorescence intensity was ranked for each of the plasmids across 4 independent experiments (FIG. 3B). High-level expression was seen in plasmids with the CAG, EF1a and CMV based promoter/enhancers.

### Example 4

Promoter/Enhancer Screening in Primary Human Cells (HBIEpC) In Vitro

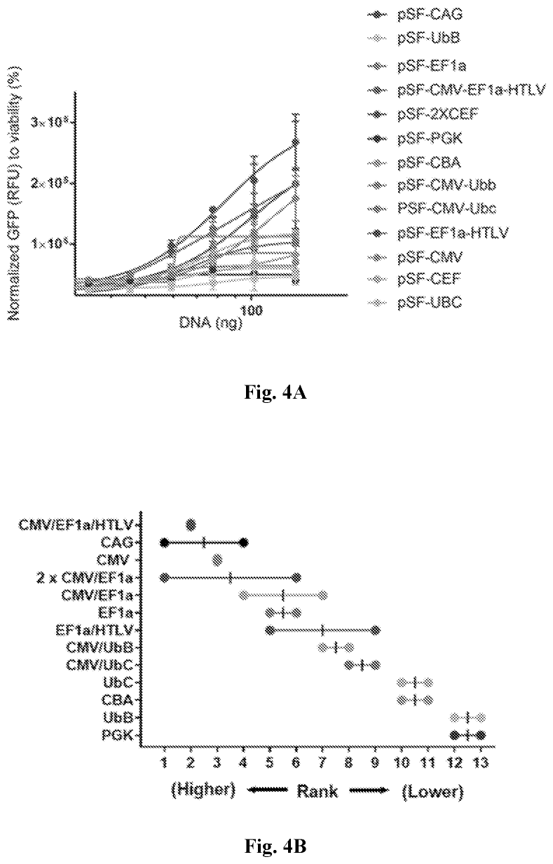

The efficacy of promotor/enhancer combinations were assessed by measuring dose-dependent expression of a green fluorescent report gene (GFP) following in vitro transfection of human primary bladder epithelial cells. Human primary bladder epithelial cells (ATCC) were seeded into 96-well plates (25,000 cells/well) 24 hours prior to transfection with the plasmids containing alternative promoter/enhancer combinations on the SnapFast backbone (pSF). Promoter/enhancer combinations studied included CAG, UbB, EF1a, CMV-EF1a-HTLV, 2×CEF, PGK, CBA, CMV-Ubb, EF1a-HTLV, CMV, CEF, and UBC. Transfection was performed using the Avalanche Transfection Reagent (EZ Biosystems) and increasing doses of plasmid DNA. At 24 hours post-transfection, fluorescence was measured on the Envision plate reader. Cell viability was measured using the AlamarBlue Cell Viability Reagent (Invitrogen). Data is represented as GFP relative fluorescence units (RFU) normalized to cell viability. As seen in FIG. 4A, the promotor/enhancer combinations that performed well in the MB49 cell line also have high levels of expression in human primary cells. Likewise, low expressers in the MB49 cell line are also showing low expression in primary cells. Total fluorescence intensity was ranked for each of the plasmids across two independent assays (FIG. 4B). Plasmids with the EF1a/HTLV, CAG, and 2×CMV/EF1a showed highest levels of expression and plasmids with UBB and PGK promoter/enhancers showed the lowest expression in the human bladder cells.

### Example 5

Measure of Retrofit Plasmids in Primary Human Bladder Epithelial Cells

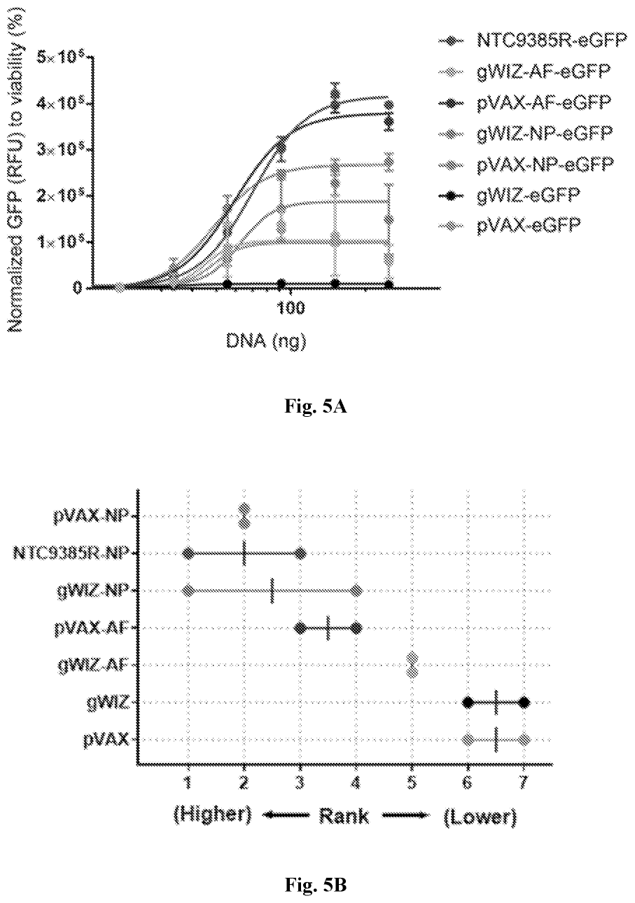

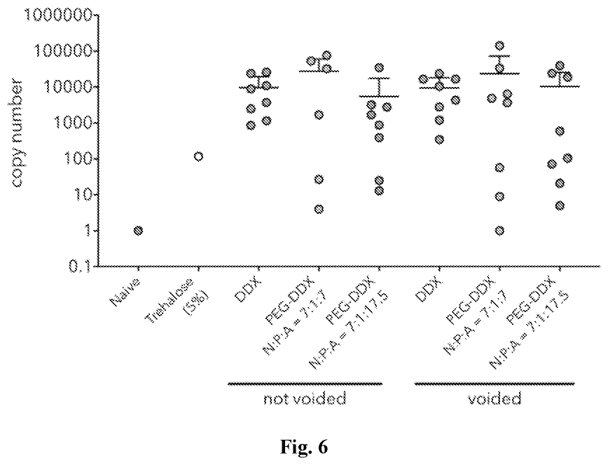

The transfection efficacy of plasmids modified (“retrofit”) to remove bacterial components was assessed using a fluorescence-based assay. Briefly, dose-dependent expression of a green fluorescent reporter gene (GFP) was measured following in vitro transfection of human primary bladder epithelial cells with a panel of plasmids retrofit to remove the bacterial components. Human primary bladder epithelial cells (Cell Applications) were seeded into 96-well plates (25,000 cells/well) 24 hours prior to transfection with the indicated plasmids (Nature Technology Corp). Transfection was performed using Avalanche Transfection Reagent (EZ Biosystems) and increasing doses of plasmid DNA. At 24 hours post-transfection, fluorescence was measured on the Envision plate reader. Cell viability was measured using the AlamarBlue Cell Viability Reagent (Invitrogen). Data is represented as GFP relative fluorescence units (RFU) normalized to cell viability (FIG. 5A). Total fluorescence intensity was ranked for each of the plasmids across 2 independent experiments (FIG. 5B). The NP plasmids are the highest expressers in the human primary bladder epithelial cells, and pVAX and gWIZ (unmodified) are the lowest expressors. The NTC9385R plasmid showed the highest expression levels of the plasmids assayed.

### Example 6

Effect of PEGylation on Bladder Transfection Efficiency In Vivo with and without Voiding of Polyplex Following the Incubation Period

mRNA expression of human PD-L1-Fc was assayed following administration of DDX and PEGylated DDX formulations to the mouse bladder. Polyplexes were prepared at an NPA ratio of 7:1:17.5 (PEG-DDX) or 7:1:7 (PEG-DDX) or 7:1:0 (DDX control). Female mice (12-16 weeks) were administered 80 μL of polyplex (20 μg [plasmid DNA) by intravesical instillation under anesthesia with isoflurane (exposure time=60 min). Bladders were voided (using a syringe and catheter through the urethra), or not, post-exposure to the polyplex and prior to waking from anesthesia. At 24 hours post-administration, bladder tissue was harvested and RNA was extracted (Qiagen RNeasy kit), following homogenization of tissue in lysis buffer. RT-qPCR was performed using 1 μg input RNA and TaqMan primer/probes that recognize codon optimized human PD-L1-Fc. Absolute quantification was performed using a standard curve of human PD-L1-Fc RNA. Data are represented as RNA copy number (FIG. 6).

### Example 7

Assessment of mRNA Expression In Vivo Using PEG Vs. Non-PEG at Different Plasmid Concentrations

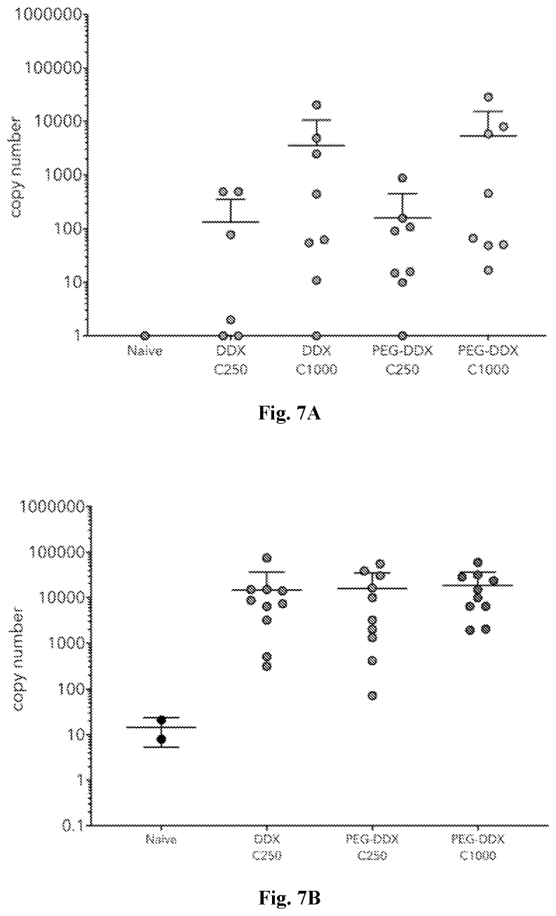

DDX and PEG-DDX polyplexes comprising human PD-L1-Fc were prepared at an NPA ratio of 7:1:9 (PEG-DDX) or 7:1:0 (DDX)). Female mice (12-16 weeks) were administered 80 μL of polyplex (0.25 mg DNA/mL=20 μg plasmid DNA; 1.0 mg DNA/mL=80 μg plasmid DNA) by intravesical instillation under anesthesia with isoflurane (exposure time=60 min). At 24 hours post-administration, bladder tissue was harvested and RNA was extracted (Qiagen RNeasy kit), following homogenization of tissue in lysis buffer. RT-qPCR was performed using 1 μg input RNA and TaqMan primer/probes that recognize codon optimized human PD-L1-Fc. Absolute quantification was performed using a standard curve of human PD-L1-Fc RNA. Data are represented as RNA copy number (FIG. 7A). In a second experiment, indicated polyplexes were prepared at NPA ratio of 7:1:3.5 (PEG-DDX) or 7:1:0 (DDX control). Female mice (12-16 weeks) were subjected to the same regimen, administration of 80 μL of polyplex (0.25 mg DNA/mL=20 μg plasmid DNA; 1.0 mg DNA/mL=80 μg plasmid DNA) by intravesical instillation under anesthesia with isoflurane (exposure time=60 min). At 24 hour post-administration, bladder tissue was harvested and RNA was extracted (Qiagen RNeasy kit), following homogenization of tissue in lysis buffer. RT-qPCR was performed using 1 μg input RNA and TaqMan primer/probes that recognize codon optimized human PD-L1-Fc. Absolute quantification was performed using a standard curve of human PD-L1-Fc RNA. Data are represented as RNA copy number (FIG. 7B). Higher expression was seen in animals dosed with 1.0 mg/DNA/ml over those administered 0.25 mg DNA/ml. PEGylation did not impair mRNA expression in the bladder 24 hours-post administration compared to the non-PEGylated formulation.

### Example 8

Expression of Human PD-L1-Fc Following Administration of PEGylated DDX Formulation in the Mouse Bladder

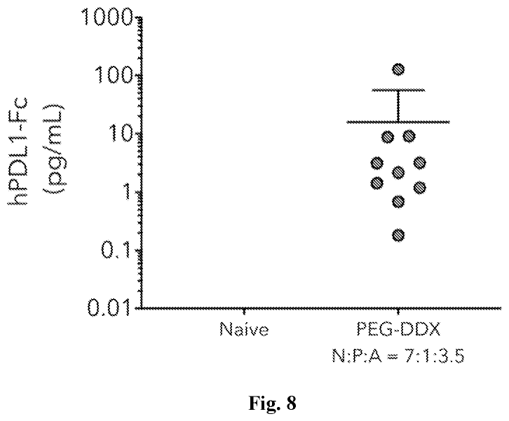

PEG-DDX polyplexes were prepared at an NPA ratio of 7:1:3.5. Female mice (12-16 weeks) were administered 80 μL of polyplex (0.125 mg DNA/mL=10 μg plasmid DNA) by intravesicalinstillation under anesthesia with isoflurane (exposure time=60 min). At 48 hours post-administration, bladder tissue was harvested and tissue was lysed by homogenization in the presence of protein lysis buffer and protease inhibitors. Human PD-L1-Fc protein was quantified using a custom-designed immunoassay (Mesoscale Discovery). Data is represented as pg/mL protein in lysate (FIG. 8). Intravesical instillation of PEG-DDX™ polyplex gave robust quantifiable protein expression in the bladder.

### Example 9

Effect of PEGylation on Polyplex Stability in Urine in Mouse Bladder.

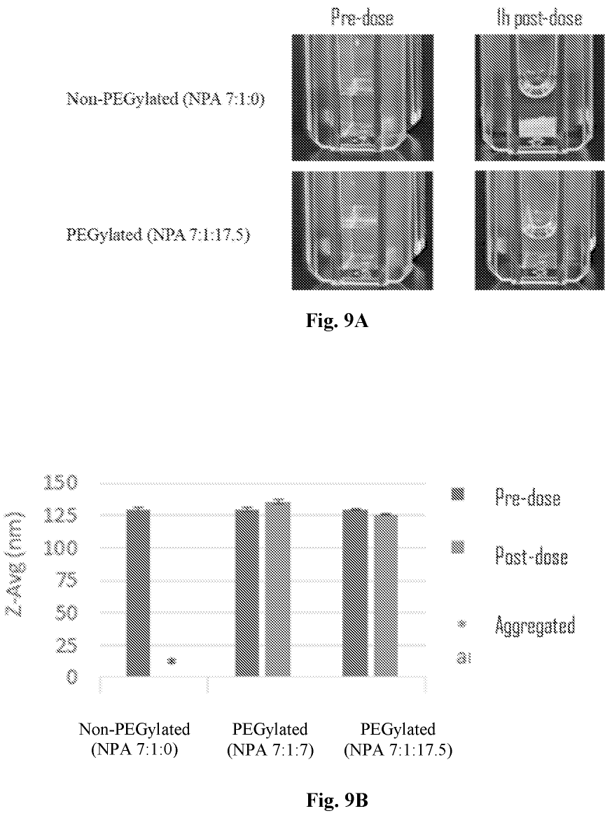

80 μL of Non-PEGylated (NPA 7:1:0) and PEGylated (NPA 7:1:7 and 7:1:17.5) DDX/DNA polyplex formulations were administered to mouse bladder at 0.25 mg DNA/mL (n=4). Formulations were incubated in the bladder for 1 h prior to bladder voiding (to collect contents of the bladder) for analysis. Samples were examined for visual appearance (FIG. 9A) and nanoparticle sizing by dynamic light scattering (FIG. 9B). Non-PEGylated polyplex were severely aggregated post voiding, with visible white clots in comparison to PEGylated polyplex, which showed no aggregation.

### Example 10

Effects of DDX-I Vs. DDX-II on hPD-L1-Fc Production In Vivo

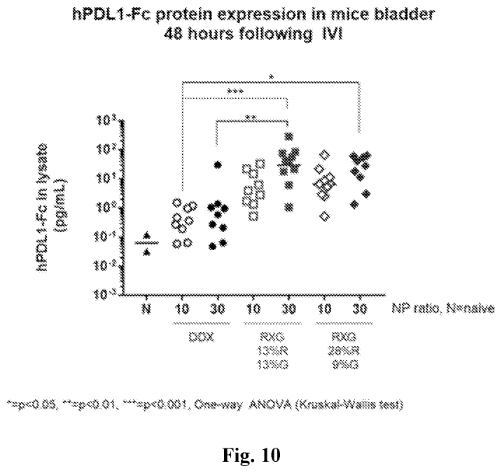

Female mice (12-16 weeks) were administered 80 μL of polyplex (1.0 mg DNA/mL=80 μg plasmid DNA) by intravesical instillation under anesthesia with isoflurane (exposure time=60 min). At 48 hours post-administration, bladder tissue was harvested and tissue was lysed by homogenization in the presence of protein lysis buffer containing protease inhibitors. Human PD-L1-Fc protein was quantified using a custom-designed immunoassay (Mesoscale Discovery). Data is represented as pg/mL protein in lysate. Data are mean+/−SD; *p<0.05; **p<0.005—one-way ANOVA with Kruskal-Wallis test. The polyplexes were prepared at NP ratio of 10:1 or 30:1 (as indicated) and DNA concentration of 1.0 mg/mL. DDX-I composition was 14% R and 3% GA. DDX-II (RXG) composition was 13% R/13% G or 28% R/9% G (as indicated). As seen in FIG. 10, the DDX-II (RXG) lead to significantly higher protein expression in vivo than DDX-I.

### Example 11

Kinetics of Protein Expression Following Administration of RXG Formulations to the Mouse Bladder

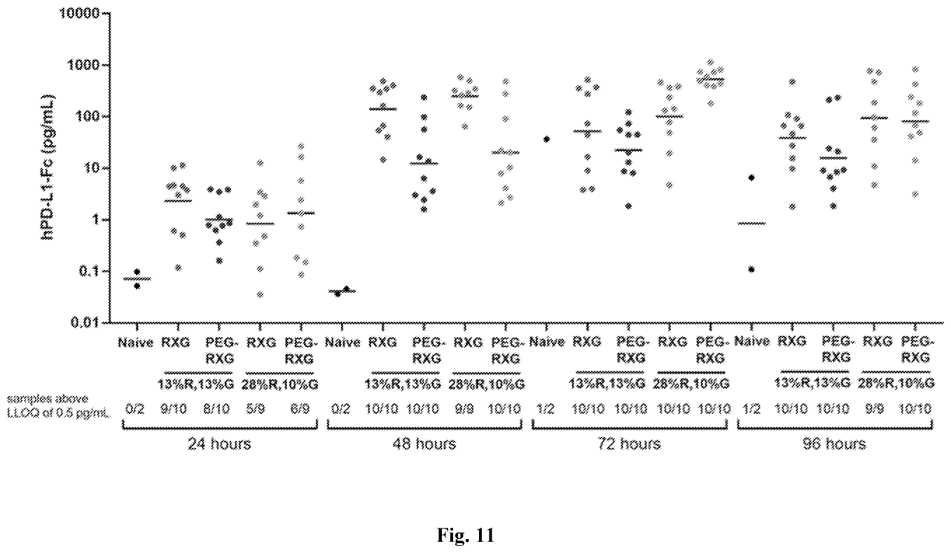

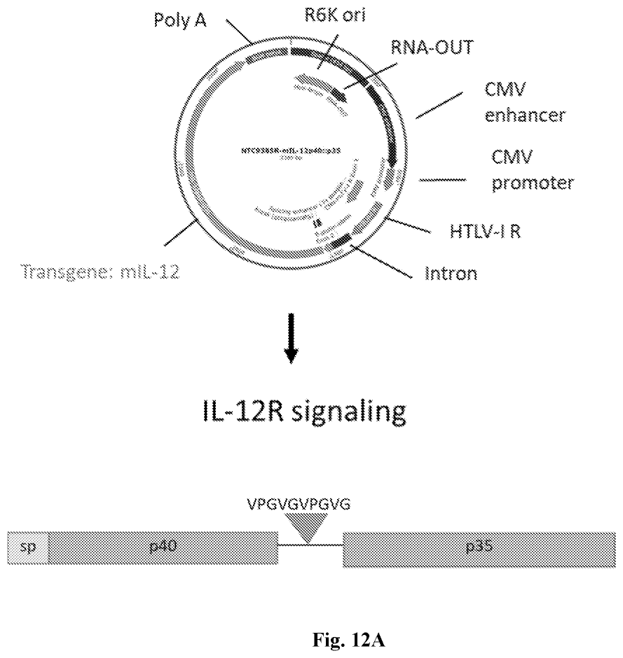

Polyplexes were prepared at NPA ratios of 10:1:5 (PEGylated) or 20:1:0 (non-PEGylated). PEGylated formulation was done in 5% trehalose+5% mannitol. Non-PEGylated formulation was done in 5% trehalose. RXG (DDX-II) composition for % R and % G are indicated. Female mice (12-16 weeks) were administered 80 μL of polyplex (1.0 mg DNA/mL=80 μg plasmid DNA) by intravesical instillation under anesthesia with isoflurane (exposure time=60 min). At the 24, 48, 72, and 96 hour points post-administration, bladder tissue was harvested and tissue was lysed by homogenization in the presence of protein lysis buffer containing protease inhibitors. Human PD-L1-Fc protein was quantified using a custom-designed immunoassay (Mesoscale Discovery). Data is represented as pg/mL protein in lysate (FIG. 11). Protein expression is high (within the ng/ml range) and is sustained up to 96 hours post-administration.

### Example 12

Plasmid DNA Constructs with Mouse IL-12 without and with the RIG-I Agonist Cassette

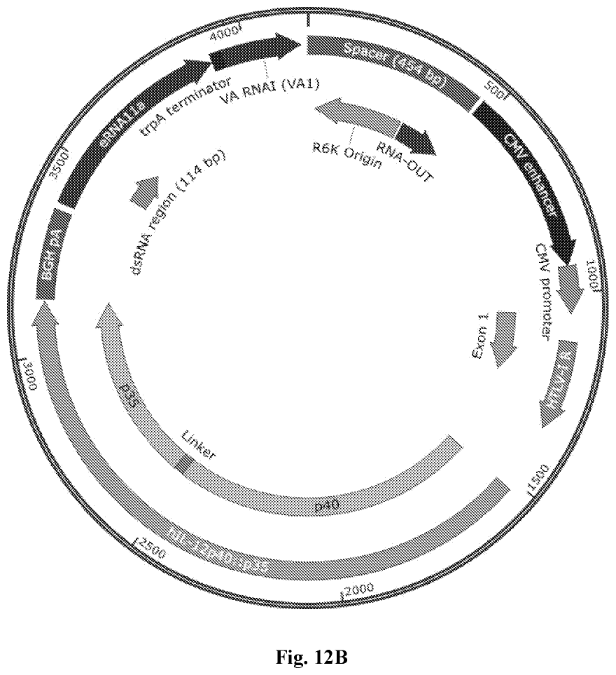

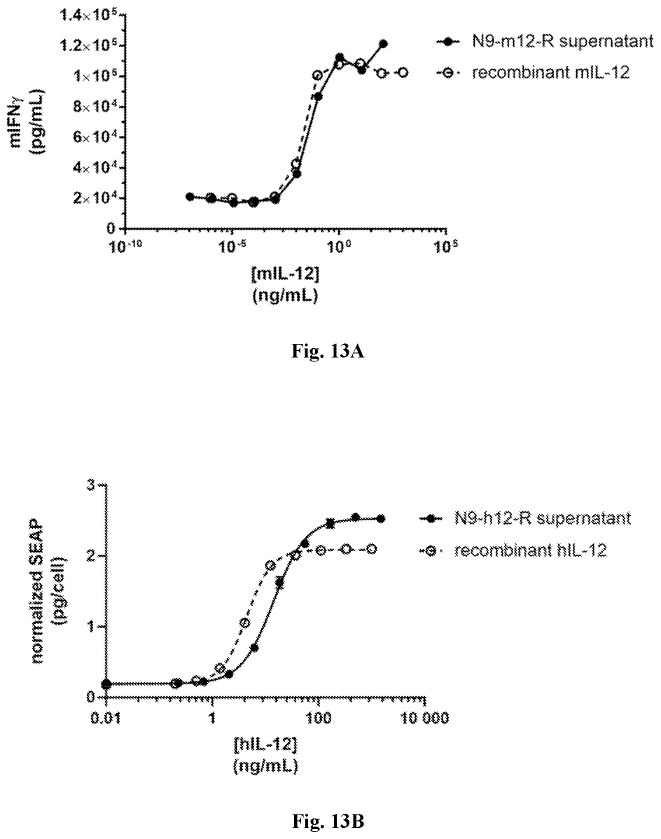

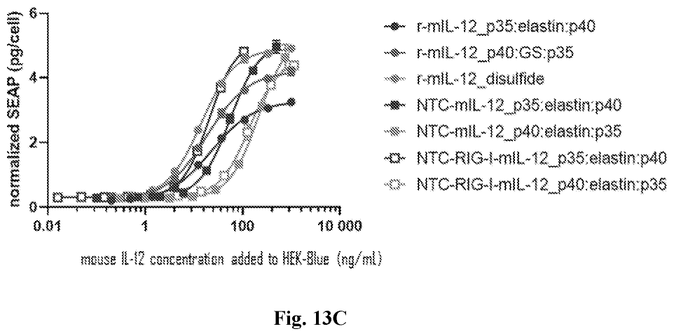

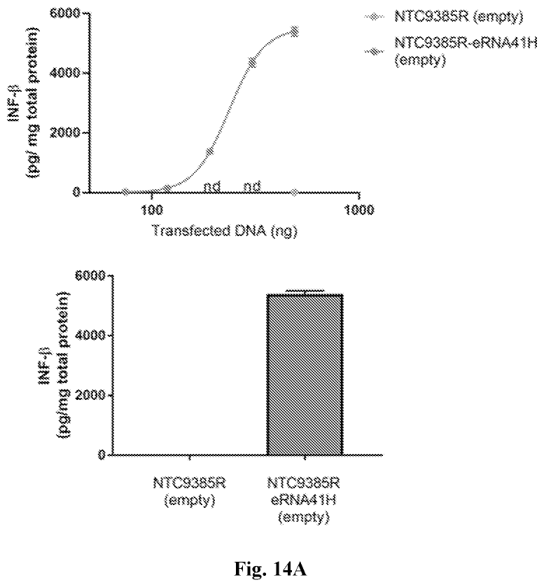

Plasmid constructs with mouse IL-12 without (FIG. 12A) and with the RIG-I agonist cassette (FIG. 12B). The mouse IL-12 transgene comprises a single open reading frame of genes for the IL-12 p40 and p35 subunits with a short elastin linker (FIG. 12A). HEK293T cells were transfected with plasmids containing the mIL-12 transgene and supernatants were harvested at 48 h post-transfection. Mouse IL-12p40p35 was quantified in the supernatant of the cells by immunoassay. Constructs yield bioactive IL-12 (FIG. 13A) Splenocytes were seeded into 96-well plates and stimulated with anti-CD3 and anti-CD28, along with increasing doses of supernatant containing mIL-12. IFNγ was measured in the splenocyte culture supernatant by ELISA at 48 h post-stimulation (FIG. 13A). HEKBlue cells were seeded into 96-well plates and stimulated with increasing doses of supernatant containing mIL-12. IL-12-mediated SEAP production was quantified in the HEKBlue supernatant, relative to a standard curve of recombinant SEAP, and data was normalized to cell number.

### Example 13

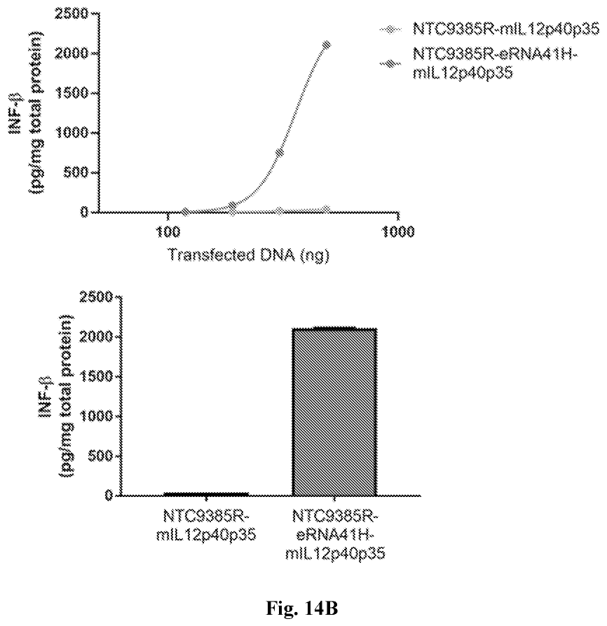

IFNβ Production In Vitro in Bladder Cancer Epithelial Cells Transfected with Plasmids with RIG-I-Agonist. (FIGS. 14A and 14B)

MB49 cells were seeded into 96-well plates (35,000 cells/well) prior to transfection with plasmids (NTC9385R-mIL12 with or without RIG-I agonists. Transfection was performed using Lipofectamine2000 (Thermofisher) and increasing doses of plasmid DNA. At 48 hours post-transfection, cell culture supernatant was collected and IFNβ production was measured by ELISA. Data was normalized to total cellular protein and represented as pg IFNβ/mg total protein.

### Example 14

mRNA Expression In Vivo Following Administration of RXG Formulation to the Mouse Bladder

A single chain mouse IL-12p40p35 open reading frame with a codon-optimized sequence was cloned into the NTC9385R or NTC9385R-eRNA41H vector backbones and expression was confirmed in vitro in MB49 cells (data not shown). Polyplexes were prepared with RXG polymer (NP20, non-PEGylated; 25% R, 10% G, 5% trehalose as an excipient).

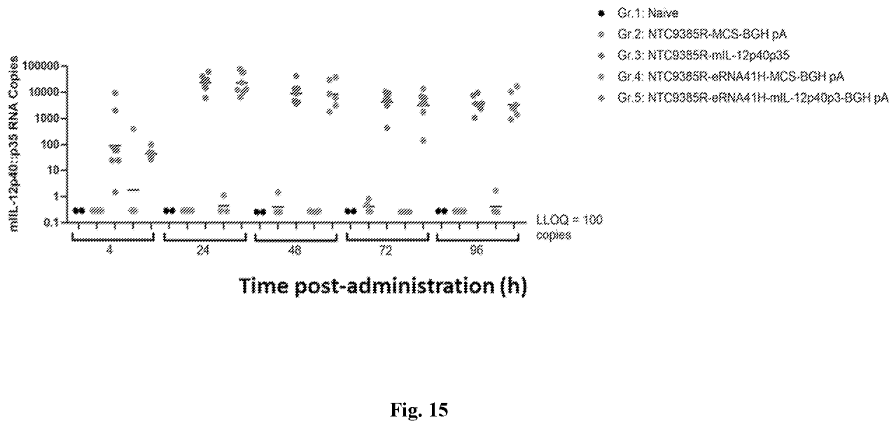

Female mice (12-16 weeks) were administered 80 μL of polyplex (1.0 mg DNA/mL=80 μg plasmid DNA) by intravesical instillation under anesthesia with isoflurane (exposure time=60 min). At the 4, 24, 48, 72, and 96 hour points post-administration, bladder tissue was harvested and RNA was extracted (Qiagen RNeasy kit), following homogenization of tissue in lysis buffer. RT-qPCR was performed using 500 ng input RNA and TaqMan primer/probes that recognize codon optimized mouse IL-12p40p35. Absolute quantification was performed using a standard curve of mouse IL-12p40p35 RNA generated by in vitro transcription. Data are represented as RNA copy number. As seen in FIG. 15, IL-12 mRNA expression was high and sustained through 96 hours. Inclusion of the RIG-I agonist did not decrease IL-12 mRNA expression.

### Example 15

Polymer Structures and Polyplex Formation

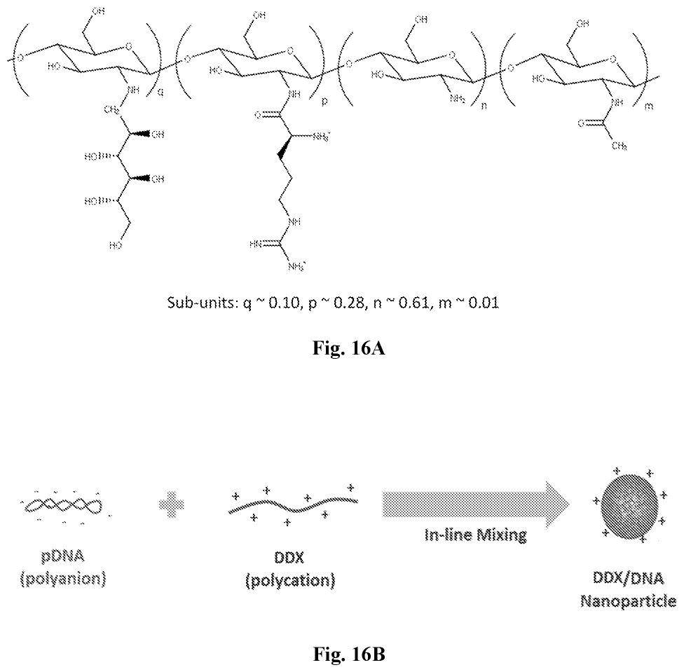

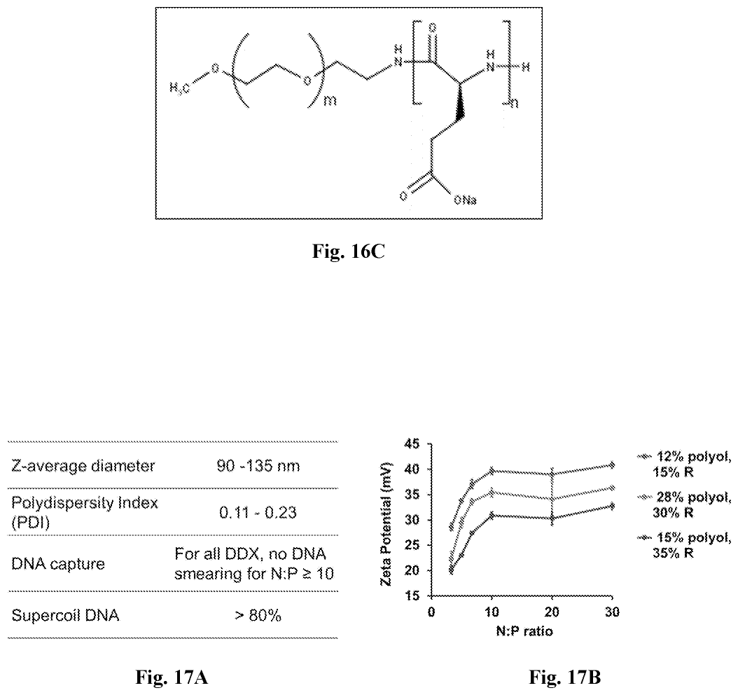

Polyplexes were formed by complexing DDX with pDNA at an amine-to-phosphate (N:P) ratio of 3 to 30, in various stabilizers for subsequent tangential flow filtration (TFF) concentration, for freezing, or freeze-drying. The structure of Dually-Derivatized Chitosan (DDX): Σ(q+p+n)=1, q=0.03−0.35, p=0.12−0.28. (FIG. 16.)

### Example 16

Physiochemical Characterization of DDX-DNA Formulations

Polyplexes were prepare at N:P ratios ranging from 3 to 30, using DDX conjugated with 12 to 28% arginine and 3 to 35% polyol. The polyplex hydrodynamic diameter (Z-average) and polydispersity index (PDI) were measured by dynamic light scattering (DLS), in 10 mM NaCl. DNA capture was determined by visual assessment of DNA release from the polyplex in a 0.8% agarose gel at pH 8 (0.5×TBE) subjected to 100 V for 1 h. DNA supercoil was quantified by agarose gel electrophoresis (Quantity One v4.6.7, Bio-Rad Laboratories) following DNA release from polyplexes by incubation with an excess of competing polyanion (Poly-(α,β)-DL-aspartic acid). Polyplex zeta potential was also measured by laser Doppler velocimetry. (FIG. 17.)

### Example 17

In Vitro Screening of Polymers

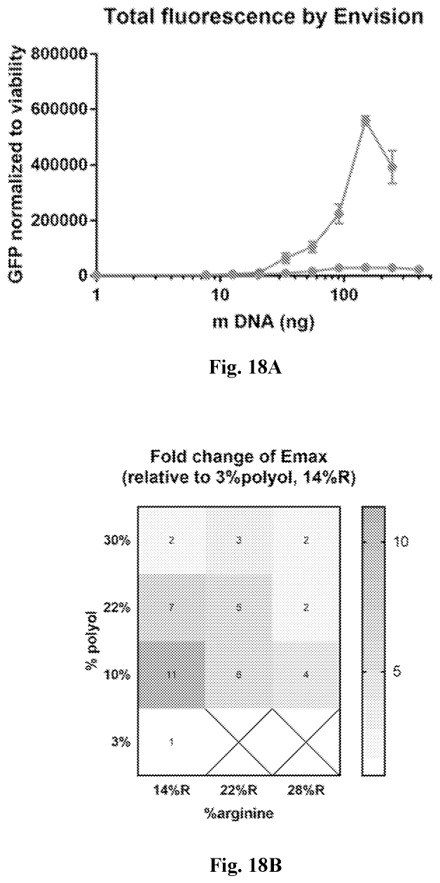

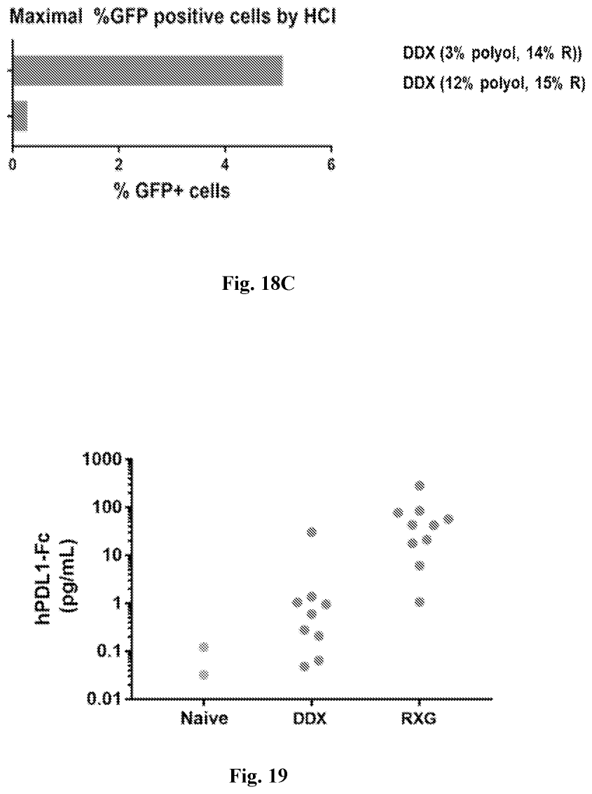

Using in vitro screening, a novel polymer with improved transfection efficacy relative to DDX (3% polyol, 14% arginine (R). Mouse urothelial carcinoma cells (MB49) were seeded into 96-well plates (35,000 cells/well) 24 hours prior to transfection with the indicated formulations. Transfection was performed with increasing doses of plasmid DNA, as indicated. At 48 hours post-transfection, fluorescence was measured on the Envision plate reader. Cell viability was measured using the AlamarBlue Cell Viability Reagent. Data is represented as GFP relative fluorescence units normalized to cell viability (FIG. 18A). B. Heatmaps represent the fold-change of maximum expression relative to DDX (3% polyol, 14% R) (FIG. 18B). The percentage of GFP positive cells was determined by high-content imaging for DDX and compared at maximum efficacy (FIG. 18C).

### Example 18

Protein Expression In Vivo Following IVI Administration of DDX.

Female mice (12-16 weeks) were administered 80 μL of polyplex (c1000=80 μg plasmid DNA) by intravesical instillation under anesthesia with isoflurane (exposure time=60 min). At 48 hours post-administration, bladder tissue was harvested and tissue was lysed by homogenization in the presence of protein lysis buffer containing protease inhibitors. Human PD-L1-Fc protein was quantified using a custom-designed immunoassay (Mesoscale Discovery). Data is represented as pg/mL protein in lysate. Improved protein expression was seen in mice following administration of DDX (12% polyol, 15% R) to the mouse bladder via IVI administration (FIG. 19).

### Example 19

Intravesical Administration of Human EG-70 in Non-Human Primates

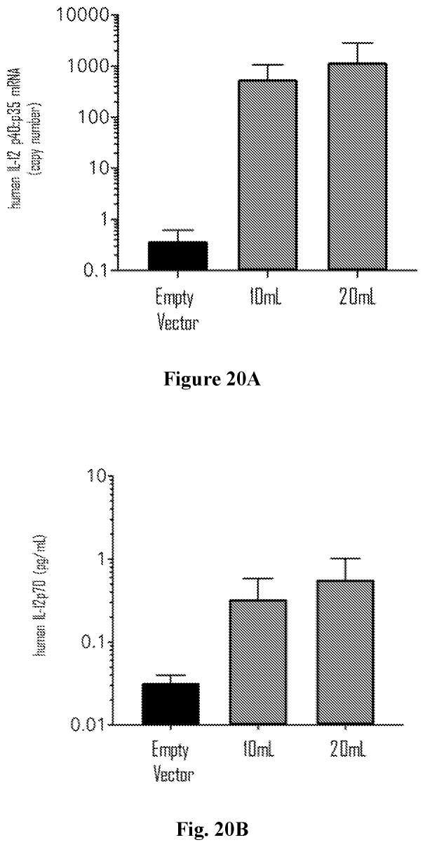

Nonhuman primates were administered 0.25 mg/ml of EG-70 comprising IL-12p40p35 by intravesical instillation under anesthesia (exposure time=60 min). Animals received 10 mL or 20 mL EG-70 or 20 mL of polyplex containing empty vector control (n=1/group, females (EG-70) or male (polyplex with empty vector DNA). At 48 hours post-administration, bladder tissue was harvested and tissue was lysed by homogenization in the presence of buffer used for RNA extraction (FIG. 20A) or protein lysis buffer containing protease inhibitors (FIG. 20B). RT-qPCR was performed using 1 μg input RNA and TaqMan primer/probes that recognize codon optimized human IL-12p40p35. Absolute quantification was performed using a standard curve of human IL-12p40p35 RNA generated by in vitro transcription (FIG. 20A). Human IL-12 protein was quantified using a using a commercially available human IL-12p70 immunoassay (Mesoscale Discovery). Data is represented as pg/mL protein in lysate (FIG. 20B). Detectable levels of mRNA and protein were seen with both intravesical administration of 10 and 20 ml treatments (corresponding to 2.5 mg or 5 mg plasmid DNA, respectively). Individual data points indicate bladder tissue fractions within the individual animal.

### Example 20

Expression of eRNA11a and VA1 in Nonhuman Primates

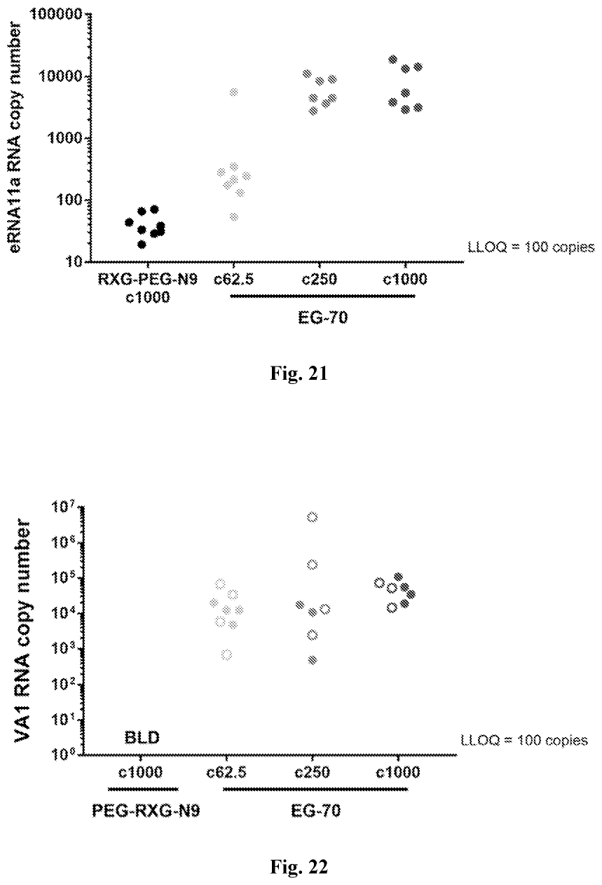

Nonhuman primates were administered 20 ml of C250 EG-70 polyplex comprising IL-12/eRNA11a/VA1, or polyplex containing empty vector control DNA, by intravesical instillation under anesthesia (exposure time=60 min). Animals received doses of EG-70 at 0.0625 mg/mL, 0.25 mg/mL or 1 mg/mL (c62.5, c250 or c1000, respectively) or 1 mg/mL (c1000) of control nanoparticle (RXG-PEG-N9; n=1/group, females (EG-70, low and mid dose) or males (EG-70, high dose or polyplex with empty vector DNA) At 48 hours post-administration, bladder tissue was harvested and tissue was lysed by homogenization in the presence of RNA extraction buffer. RT-qPCR was performed using 1 μg input RNA and TaqMan primer/probes that recognize eRNA11a or VA1. Absolute quantification was performed using standard curves of human eRNA11a RNA (FIG. 21), and human VA1 RNA (FIG. 22) generated by in vitro transcription (individual data points indicate bladder tissue fractions within the individual animal).

### Example 21

Assessment of Anti-Tumor Activity in a Murine Model of Bladder Cancer

To evaluate the anti-tumor activity of mEG-70-prototype nanoparticles, an orthotopic model of murine bladder cancer was used. Briefly, disease was established by pretreating murine bladders with poly-L-lysine to promote desquamation of the superficial urothelial layer and facilitate cancer cell implantation. Urothelial carcinoma cells that stably overexpress the luciferase gene (MB49-Luc) were subsequently instilled into murine bladders (100,000 cells per mouse) and luciferase expression was confirmed at Day 12 post-instillation using an In Vivo Imaging System (IVIS). The intensity of the bioluminescent signal was used to randomize animals into treatment groups. In addition, animals without a positive bioluminescent signal were excluded from the study. Mice received two weekly intravesical administrations of nanoparticles at Day 13 and Day 20 post instillation. This dosing regimen was selected based on the assessment of protein expression kinetics. An additional group of animals received a sham procedure with administration of the nanoparticle vehicle, trehalose (5%). The experiment was terminated on Day 29 to measure bladder weight as a function of tumor burden in the bladder.

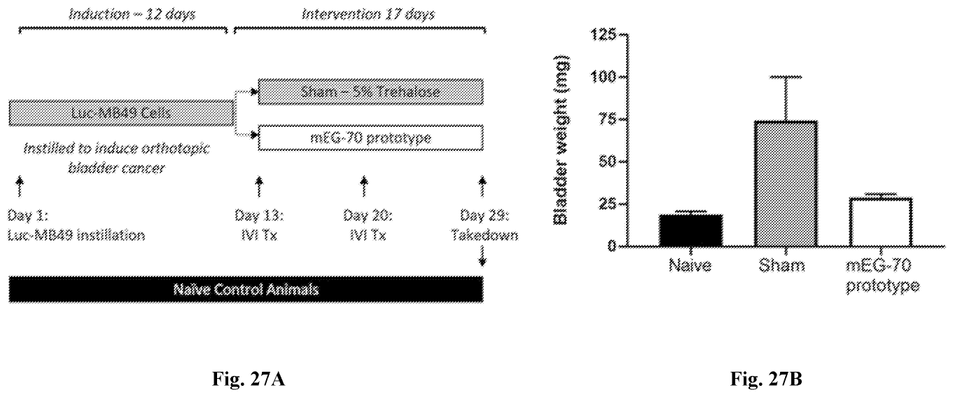

As shown in FIGS. 27A and 27B sham-treated tumor-bearing animals exhibited an average bladder weight of −75 mg, while animals treated with mEG-70-prototype at a dose of 20 μg plasmid DNA had bladder weights nearly indistinguishable from naïve animals (˜20 mg).

### Example 22

Kinetics of Mouse IL-12 mRNA Expression in Murine Bladder

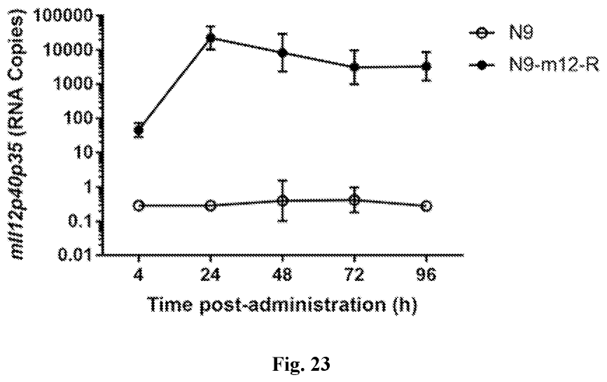

To evaluate the kinetics of codon-optimized mouse Il12p40p35 gene expression in murine bladder, healthy female C57Bl/6J mice (12-16 weeks) received a single intravesical instillation (IVI) of RXG nanoparticles comprising IL-12/eRNA11a/VA1, or polyplex containing empty vector control DNA, by intravesical instillation under anesthesia (exposure time=60 min). Animals received doses of mEG-70 prototype or control nanoparticle (RXG-PEG-N9) at 0.25 mg/mL, c250. At the indicated times post-administration, bladder tissue was harvested and tissue was lysed by homogenization in the presence of an RNA extraction buffer. RT-qPCR was performed using 1 μg input RNA and TaqMan primer/probes that codon-optimized mIL-12p40p35. Absolute quantification was performed using a standard curve of mouse Il12p40p35 RNA generated by in vitro transcription (FIG. 23).

As shown in FIG. 23, mouse Il12p40p35 mRNA levels in bladder tissue were detectable as early as 4 h post-administration and expression was sustained up to 96 h after dosing in mice that had received mEG-70-prototype nanoparticles. As expected, mice that received nanoparticles containing plasmid without the IL-12 transgene (N9, negative control) did not have any detectable Il12p40p35 mRNA (<2 copies).

### Example 23

Kinetics of Mouse IL-12p70 Protein Expression in the Mouse Bladder

To evaluate the kinetics of mouse IL12p70 protein expression in murine bladder, healthy female C57Bl/6J mice (12-16 weeks) received a single IVI of 20 μg mEG-70-prototype nanoparticles. Bladder tissue was harvested at the indicated times and tissue lysates were prepared for mouse IL12p70 immunoassay on the Mesoscale Discovery (MSD) platform.

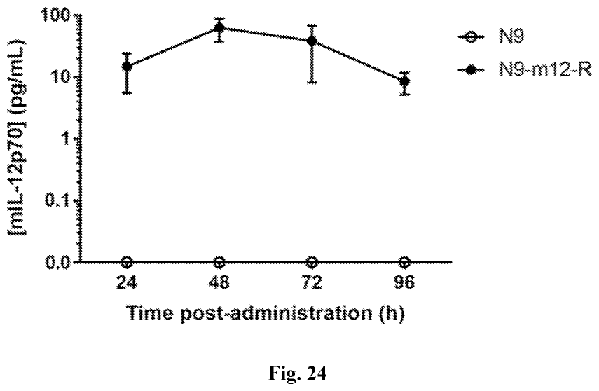

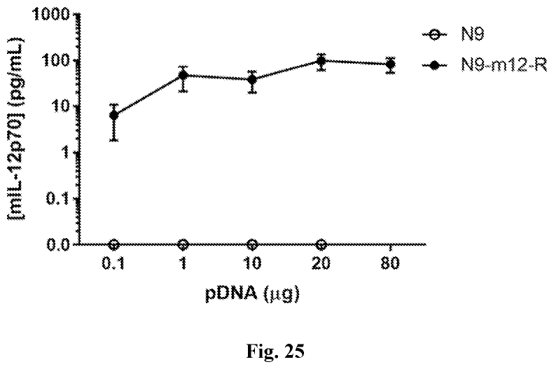

As shown in FIG. 24, mice that received one IVI of mEG-70-prototype nanoparticles showed detectable levels of IL-12p70 protein as early as 24 h post-administration. Protein expression was highest at 48 h and was detected as late as 96 h after dosing. There was no detectable IL-12p70 in the bladder tissue of mice that had received an administration of nanoparticles containing N9 plasmid DNA (negative control).

### Example 24

Dose Response of Mouse IL-12p40p35 Protein Expression in the Mouse Bladder

To evaluate if there is dose-dependent expression of mouse IL12p70 protein expression in murine bladder, healthy female C57Bl/6J mice (12-16 weeks) received a single IVI of mEG 70 prototype nanoparticles at doses between 0.1-80 μg plasmid DNA. The negative control was dosed up to 20 μg. Bladder tissue was harvested at 48 h post-administration and tissue lysates were prepared for mouse IL12p70 immunoassay on the MSD platform.

As shown in FIG. 25, mice that received one IVI of mEG-70-prototype nanoparticles showed low level and frequency (25% mice) of IL-12p70 protein at the lowest dose of N9-m12-R (0.1 μg). Protein expression was detected at comparable levels for all doses ranging from 1 80 μg. There was no detectable IL-12p70 in the bladder tissue of mice that had received an administration of nanoparticles containing N9 plasmid DNA (negative control).

### Example 25

PEGylated Nanoparticles Expression In Vivo

A study was conducted to assess any potential impact of PEGylation on mouse IL-12p70 protein expression. Briefly, female C57Bl/6J mice (12-16 weeks) received a single administration of nanoparticles containing 20 μg plasmid DNA. Mice received a single IVI of mEG-70 or mEG-70-prototype nanoparticles. Bladder tissue was harvested at 24 hours to 7 days post-administration and tissue protein lysates were generated for the quantification of IL 12p70 protein by immunoassay.

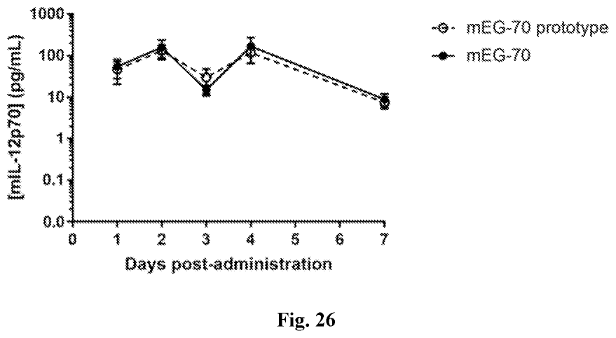

As shown in FIG. 26, mice that received a single IVI of either mEG-70 prototype or mEG-70 showed comparable levels and kinetics of mouse IL-12p70 protein expression.

### Example 26

Human Clinical Study in NMIBC

Bladder cancer is the fourth and tenth most common malignancy among men and women in the United States (US), respectively (American Cancer Society 2019). Non-muscle invasive bladder cancer (NIMBC) is generally managed with surgical resection (TURBT) followed often by a single dose of intravesical chemotherapy within 24 hours (gemcitabine or mitomycin) to reduce the recurrence rate by 35% (Sylvester et al, 2016).

After confirmation of the presence of bladder cancer from pathology, physicians develop a continued treatment plan, frequently involving BCG therapy. Despite significant adverse effects, and a 30% to 40% failure rate, intravesical immunotherapy with BCG is the mainstay treatment used to prevent recurrence and/or progression in patients with high grade (Ta and above) NMIBC. BCG is often given in a second maintenance course to achieve a disease-free state, even though patients with BCG-unresponsive NMIBC are extremely unlikely to benefit from further therapy with BCG, and therefore represent a unique population for the study of new therapies (Jarow et al, 2015).

In the absence of pharmacologic intervention or cystectomy, BCG-unresponsive NMIBC, with or without resected disease, will persist and progress. To date, there are no effective therapies available for patients who have failed BCG, as gemcitabine and mitomycin often given post TURBT are not effective salvage agents. Therefore, the treatment for BGC-unresponsive disease (regardless if BCG refractory or relapsed) is radical cystectomy to surgically remove all tumor and ensure disease-free survival. The fact that there are few treatment options available for NMIBC, and patients continue to have radical organ removal for early stage disease describes a truly great unmet medical need. More effective treatments that are active in refractory patients are desperately needed in NMIBC.

In an exemplary embodiment of the present invention, the therapeutic nucleic acid comprises a 4156 bp plasmid DNA (pDNA) comprised of a codon optimized human interleukin-12 gene termed opt-hIL-12 (SEQ ID NO:7 linked to a constitutively active cytomegalovirus (CMV) promoter on a NTC9385R backbone with an antibiotic-free selection marker based on sucrose (RNA-OUT), as set forth in Table below:

TABLE 1

| | |
|---|---|
| CCGCCTAATG AGCGGGCTTT TTTTTGGCTT GTTGTCCACA ACCGTTAAAC | 50 |
| CTTAAAAGCT TTAAAAGCCT TATATATTCT TTTTTTTCTT ATAAAACTTA | 100 |
| AAACCTTAGA GGCTATTTAA GTTGCTGATT TATATTAATT TTATTGTTCA | 150 |
| AACATGAGAG CTTAGTACGT GAAACATGAG AGCTTAGTAC GTTAGCCATG | 200 |
| AGAGCTTAGT ACGTTAGCCA TGAGGGTTTA GTTCGTTAAA CATGAGAGCT | 250 |
| TAGTACGTTA AACATGAGAG CTTAGTACGT ACTATCAACA GGTTGAACTG | 300 |
| CTGATCCACG TTGTGGTAGA ATTGGTAAAG AGAGTCGTGT AAAATATCGA | 350 |
| GTTCGCACAT CTTGTTGTCT GATTATTGAT TTTTGGCGAA ACCATTTGAT | 400 |
| CATATGACAA GATGTGTATC TACCTTAACT TAATGATTTT GATAAAAATC | 450 |
| ATTAGGTACC CCGGCTCTAG TTATTAATAG TAATCAATTA CGGGGTCATT | 500 |
| AGTTCATAGC CCATATATGG AGTTCCGCGT TACATAACTT ACGGTAAATG | 550 |
| GCCCGCCTGG CTGACCGCCC AACGACCCCC GCCCATTGAC GTCAATAATG | 600 |
| ACGTATGTTC CCATAGTAAC GCCAATAGGG ACTTTCCATT GACGTCAATG | 650 |
| GGTGGAGTAT TTACGGTAAA CTGCCCACTT GGCAGTACAT CAAGTGTATC | 700 |
| ATATGCCAAG TACGCCCCCT ATTGACGTCA ATGACGGTAA ATGGCCCGCC | 750 |
| TGGCATTATG CCCAGTACAT GACCTTATGG GACTTTCCTA CTTGGCAGTA | 800 |
| CATCTACGTA TTAGTCATCG CTATTACCAT GGTGATGCGG TTTTGGCAGT | 850 |
| ACATCAATGG GCGTGGATAG CGGTTTGACT CACGGGGATT TCCAAGTCTC | 900 |
| CACCCCATTG ACGTCAATGG GAGTTTGTTT TGGCACCAAA ATCAACGGGA | 950 |
| CTTTCCAAAA TGTCGTAACA ACTCCGCCCC ATTGACGCAA ATGGGCGGTA | 1000 |
| GGCGTGTACG GTGGGAGGTC TATATAAGCA GAGCTCGTTT AGTGAACCGT | 1050 |
| CAGATCGCCT GGAGACGCCA TCCACGCTGT TTTGACCTCC ATAGAAGACA | 1100 |
| CCGGGACCGA TCCAGCCTCC GCGGCTCGCA TCTCTCCTTC ACGCGCCCGC | 1150 |
| CGCCCTACCT GAGGCCGCCA TCCACGCCGG TTGAGTCGCG TTCTGCCGCC | 1200 |
| TCCCGCCTGT GGTGCCTCCT GAACTGCGTC CGCCGTCTAG GTAAGTTTAA | 1250 |
| AGCTCAGGTC GAGACCGGGC CTTTGTCCGG CGCTCCCTTG GAGCCTACCT | 1300 |
| AGACTCAGCC GGCTCTCCAC GCTTTGCCTG ACCCTGCTTG CTCAACTCTA | 1350 |
| GTTCTCTCGT TAACTTAATG AGACAGATAG AAACTGGTCT TGTAGAAACA | 1400 |
| GAGTAGTCGC CTGCTTTTCT GCCAGGTGCT GACTTCTCTC CCCTGGGCTT | 1450 |
| TTTTCTTTTT CTCAGGTTGA AAAGAAGAAG ACGAAGAAGA CGAAGAAGAC | 1500 |
| AAACCGTCGT CGACGCCGCC ACCATGTGCC ATCAGCAACT TGTCATCTCC | 1550 |
| TGGTTCTCCC TCGTGTTCCT GGCCTCCCCT CTTGTCGCGA TTTGGGAGCT | 1600 |
| GAAGAAAGAT GTGTACGTCG TGGAACTCGA CTGGTACCCG GACGCCCCCG | 1650 |
| GGGAAATGGT GGTGCTCACT TGTGATACTC CCGAAGAGGA TGGAATTACC | 1700 |
| TGGACCCTCG ATCAGTCCTC CGAGGTCTTG GGATCCGGCA AAACTCTGAC | 1750 |
| CATCCAAGTC AAGGAATTCG GCGACGCGGG GCAGTACACC TGTCACAAGG | 1800 |
| GCGGAGAAGT GCTGTCGCAC TCACTCCTGC TCCTTCACAA AAAGGAGGAC | 1850 |
| GGCATCTGGT CGACCGACAT CCTGAAGGAC CAGAAGGAAC CCAAGAACAA | 1900 |
| GACCTTTCTG CGCTGCGAGG CCAAGAACTA TTCGGGAAGG TTCACCTGTT | 1950 |
| GGTGGCTGAC TACCATCTCC ACCGACCTGA CTTTCTCCGT GAAGTCCTCT | 2000 |
| CGGGGTTCGA GCGACCCGCA GGGTGTTACG TGCGGTGCTG CAACCCTGTC | 2050 |
| CGCGGAGAGA GTGCGGGGGG ACAACAAGGA ATACGAGTAC TCAGTGGAAT | 2100 |
| GCCAGGAAGA TAGCGCCTGC CCTGCCGCCG AAGAGTCCCT GCCGATTGAA | 2150 |
| GTCATGGTGG ACGCAGTGCA TAAGTTGAAA TATGAGAACT ACACCTCGTC | 2200 |
| GTTCTTCATC CGGGACATCA TCAAGCCTGA CCCCCCTAAG AATCTGCAGC | 2250 |
| TCAAGCCCCT CAAGAACTCC AGACAGGTCG AAGTGTCCTG GGAGTACCCA | 2300 |
| GATACGTGGA GCACACCGCA CTCGTACTTC TCCTTGACCT TCTGCGTCCA | 2350 |
| AGTGCAGGGA AAGTCCAAAC GGGAGAAGAA GGACCGCGTG TTCACTGATA | 2400 |
| AGACTTCCGC TACTGTGATC TGCCGCAAAA ACGCCAGCAT CAGCGTGCGC | 2450 |
| GCGCAAGATA GATACTACTC AAGCTCTTGG TCCGAATGGG CGTCCGTGCC | 2500 |
| ATGCTCGGTG CCCGGCGTGG GCGTGCCTGG AGTGGGAGCC CGGAACTTGC | 2550 |
| CGGTGGCCAC CCCTGACCCC GGAATGTTCC CTTGCCTGCA CCACTCCCAA | 2600 |
| AACCTTCTGA GGGCTGTGTC CAACATGCTG CAGAAGGCTC GGCAGACCCT | 2650 |
| GGAATTCTAC CCCTGCACCT CCGAGGAGAT CGACCACGAA GATATTACCA | 2700 |
| AGGACAAGAC CTCAACCGTG GAAGCCTGCC TGCCCCTGGA ACTGACCAAG | 2750 |
| AACGAATCGT GCCTGAATAG CCGGGAAACC TCCTTCATCA CCAACGGCTC | 2800 |
| CTGCCTGGCC TCACGAAAGA CCAGCTTTAT GATGGCCCTG TGCCTGAGCT | 2850 |
| CGATCTAGGA GGACCTGAAG ATGTACCAGG TCGAGTTCAA GACTATGAAC | 2900 |
| GCCAAGCTGC TGATGGATCC GAAGCGGCAG ATCTTCTTGG ACCAGAATAT | 2950 |
| GCTGGCAGTG ATCGACGAGC TGATGCAGGC CCTCAACTTC AACTCCGAGA | 3000 |
| CTGTGCCGCA AAAGTCGAGC CTGGAGGAAC CGGACTTCTA CAAGACCAAG | 3050 |
| ATCAAGTTAT GTATTCTCCT GCACGCGTTT AGGATTCGCG CCGTGACCAT | 3100 |
| TGATAGAGTG ATGTCCTACC TGAACGCCAG CTGAGAATTC CTGTGCCTTC | 3150 |
| TAGTTGCCAG CCATCTGTTG TTTGCCCCTC CCCCGTGCCT TCCTTGACCC | 3200 |
| TGGAAGGTGC CACTCCCACT GTCCTTTCCT AATAAAATGA GGAAATTGCA | 3250 |
| TCGCATTGTC TGAGTAGGTG TCATTCTATT CTGGGGGGTG GGGTGGGGCA | 3300 |
| GGACAGCAAG GGGGAGGATT GGGAAGACAA TAGCAGGCAT GCTGGGGATG | 3350 |
| CGGTGGGCTC TATGGCCCGG GACGGCCGCT AGCACCGTTG GTTTCCGTAG | 3400 |
| TGTAGTGGTT ATCACGTTCG CCTAACACGC GAAAGGTCCC CGGTTCGAAA | 3450 |
| CCGGGCACTA CAAACCAACA ACGTTAAAAA ACAGGTCCTC CCCATACTCT | 3500 |
| TTCATTGTAC ACACCGCAAG CTCGACAATC ATCGGATTGA AGCATTGTCG | 3550 |
| CACACATCTT CCACACAGGA TCAGTACCTG CTTTCGCTTT TAACCAAGGC | 3600 |
| TTTTCTCCAA GGGATATTTA TAGTCTCAAA ACACACAATT ACTTTACAGT | 3650 |
| TAGGGTGAGT TTCCTTTTGT GCTGTTTTTT AAAATAATAA TTTAGTATTT | 3700 |
| GTATCTCTTA TAGAAATCCA AGCCTATCAT GTAAAATGTA GCTAGTATTA | 3750 |
| AAAAGAACAG ATTATCTGTC TTTTATCGCA CATTAAGCCT CTATAGTTAC | 3800 |
| TAGGAAATAT TATATGCAAA TTAACCGGGG CAGGGGAGTA GCCGAGCTTC | 3850 |
| TCCCACAAGT CTGTGCGAGG GGGCCGGCGC GGGCCTAGAG ATGGCGGCGT | 3900 |
| CGGATCGGCC AGCCCGCCTA ATGAGCGGGC TTTTTTTTCT TAGGGTGCAA | 3950 |
| AAGGAGAGCC TGTAAGCGGG CACTCTTCCG TGGTCTGGTG GATAAATTCG | 4000 |
| CAAGGGTATC ATGGCGGACG ACCGGGGTTC GAGCCCCGTA TCCGGCCGTC | 4050 |
| CGCCGTGATC CATGCGGTTA CCGCCCGCGT GTCGAACCCA GGTGTGCGAC | 4100 |
| GTCAGACAAC GGGGGAGTGC TCCTTTTGGC TTCCTTCCCC TACCGGGGCC | 4150 |
| GCTAGC | 4156 |

The R6K origin of replication restricts plasmid replication to a specific strain of Escherichia coli (E. coli). The opt-hIL12 gene encodes the two sub-units (p40 and p35) of the cytokine protein, IL-12. To ensure 1:1 stoichiometry of the subunits, the EG-70 plasmid was designed to contain a single open reading frame (ORF) to monomerize p40 to p35 by the addition of a short repeating elastin linker sequence. The plasmid is also comprised of genes for eRNA11a (an immunostimulatory double-stranded ribonucleic acid [dsRNA]) and adenovirus VA RNA1. The two RNA products of these genes stimulate the RIG-I pathway, which recruits more immune cells to the local tissue. In a further embodiment, this therapeutic nucleic acid is packaged in a dually-derivatized chitosan polymer functionalized with arginine and glucose and coated with a detachable PEG-b-PLE excipients, to form the pharmaceutical composition EG-70. The composition is formulated as an aqueous nanoparticle dispersion in 1% w/w mannitol solution, filter sterilized, lyophilized to a dry powder, and stored at 4° C. The average particle size of the nanoparticle dispersion is in the 75-175 nanometer range.

This study will evaluate the safety of intravesical administration of EG-70 and its effect on bladder tumors in NMIBC patients who have failed BCG therapy and are awaiting radical cystectomy. The study will be a classic dose escalation trial where 3 patients are treated in each cohort. The initial dose of EG-70 will be based on the nonclinical toxicology data as well as the nonclinical efficacy data, and will be at least ⅕ of the minimal toxic dose seen in the GLP-toxicology study. Projected Phase I dose escalations will be in up to M2-log increments for successive cohorts treated without dose-limiting toxicity (DLT).

### EQUIVALENTS

All publications, patents, and patent applications mentioned in this specification are herein incorporated by reference in the entirety and for all purposes and to the same extent as if each individual publication, patent, or patent application was specifically and individually indicated to be incorporated by reference. The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

1. A composition comprising a derivatized-chitosan nucleic acid polyplex comprising amino-functionalized chitosan and at least one therapeutic nucleic acid construct encoding interleukin-12 (IL-12), wherein said derivatized-chitosan nucleic acid polyplex further comprises a reversible coating comprising one or more polyanion-containing block co-polymers having at least one polyanionic anchor region and at least one hydrophilic tail region.

2. The composition according to claim 1, wherein said amino-functionalized chitosan further comprises or is functionalized with a hydrophilic polyol.

3. The composition according to claim 1, wherein said amino-functionalized chitosan comprises arginine.

4. The composition according to claim 2, wherein said hydrophilic polyol is glucose or gluconic acid.

5. The composition according to claim 1, wherein the polyanion-containing block co-polymer is a linear diblock or triblock co-polymer.

6. The composition according to claim 1, wherein said polyplex further comprises nucleic acid encoding an additional immunostimulatory molecule selected from the group consisting of IFN-1 activators and immune checkpoint inhibitors.

7. The composition according to claim 1, wherein said therapeutic nucleic acid construct further comprises a nucleic acid encoding an additional immunostimulatory molecule selected from the group consisting of IFN-1 activators and immune checkpoint inhibitors.

8. The composition according to claim 6, wherein said IFN-1 activator is selected from the group consisting of RIG-I agonists, STING agonists and TLR 7/9 agonists.

9. The compositions according to claim 8, wherein said RIG-I agonist is selected from the group consisting of eRNA11a, VA RNA1, eRNA41H, MK4621, SLR10, SLR14, and SLR20, and more preferably selected from the group consisting of eRNA41H or eRNA11a.

10. The composition according to claim 1, wherein said therapeutic nucleic acid construct is comprised within a plasmid selected from the group consisting of: gWIZ, pVAX, NTC8685 or NTC9385R, optionally wherein said therapeutic nucleic acid construct further comprises an expression control element selected from the group consisting of CMV, EF1a, CMV/EF1a, CAG, and CMV/EF1a/HTLV promoters.

11. The composition according to claim 1, wherein said therapeutic nucleic acid construct further comprises a synthetic Beta-globin-based intron, optionally wherein said therapeutic nucleic acid construct further comprises a HTLV-IR.

12. The composition according to claim 1, wherein said therapeutic nucleic acid construct further comprises kanamycin selection or sucrose-based selection element, optionally wherein said therapeutic nucleic acid construct further comprises a pUC or R6K origin of replication.

13. The composition according to claim 1, wherein said therapeutic nucleic acid construct encoding IL-12 comprises SEQ ID NO: 7.

14. A method for the localized expression of IL-12 in a mucosal tissue in a patient in need thereof, comprising administering to said patient a therapeutically-effective amount of a pharmaceutical composition comprising the derivatized-chitosan nucleic acid polyplex according to claim 1.

15. A method for inhibiting the growth of a mucosal cancer cell in a patient in need thereof, comprising administering to said patient a therapeutically effective amount of a pharmaceutical composition comprising the derivatized-chitosan nucleic acid polyplex according to claim 1.

16. A method of treating bladder cancer in a patient in need thereof, comprising administering to said patient a therapeutically effective amount of a pharmaceutical composition comprising the derivatized-chitosan nucleic acid polyplex according to claim 1.

17. The method according to claim 14, further comprising simultaneous or sequential administration of an additional immunostimulatory molecule selected from the group consisting of IFN-1 activators and immune checkpoint inhibitors.

18. The method according to claim 17, wherein said IFN-1 activator is selected from the group consisting of RIG-I agonists, STING agonists and TLR 7/9 agonists.

19. The method according to claim 17, wherein said IFN-1 agonist and/or said immune checkpoint inhibitor is encoded by the same or a different therapeutic nucleic acid construct.

20. The method according to claim 17, wherein said IFN-1 agonist and/or said immune checkpoint inhibitor is separately administered.

21. The method according to claim 18, wherein said IFN-1 agonist is a RIG-I agonist; preferably wherein said RIG-I agonist is selected from the group consisting of eRNA11a, VA RNA1, eRNA41H, MK4621, SLR10, SLR14, and SLR20, and more preferably selected from the group consisting of eRNA41H or eRNA11a.

22. The method according to claim 20, wherein said immune checkpoint inhibitor is selected from the group consisting of ipilimumab, tremelimumab, nivolumab, atezolizumab and/or pembrolizumab.

23. A composition comprising a nucleic acid polyplex comprising a cationic polymer and/or lipid, a therapeutic nucleic acid construct encoding interleukin-12 (IL-12), and a therapeutic nucleic acid construct comprising a nucleic acid encoding at least one RIG-I agonist, wherein the therapeutic nucleic acid constructs encoding IL-12 and RIG-I are the same or different nucleic acid constructs.

24. The composition according to claim 23, wherein said RIG-I agonist is selected from the group consisting of eRNA11a, VA RNA1, eRNA41H, MK4621, SLR10, SLR14, and SLR20, and more preferably selected from the group consisting of eRNA41H, eRNA11a.

25. The composition according to claim 23, wherein said cationic polymer is selected from the group consisting of polyethyleneimine (PEI), PAMAM, polylysine (PLL), polyarginine, chitosan, and derivatives thereof.

26. The composition according to claim 25, wherein the cationic polymer comprises a derivatized chitosan, preferably an amino-functionalized chitosan.

27. The composition according to claim 26, wherein said amino-functionalized chitosan comprises arginine and further comprises, or is functionalized with, a hydrophilic polyol, e.g. gluconic acid or glucose.

28. The composition according to claim 25, wherein the nucleic acid polyplex further comprises a reversible coating comprising one or more polyanion-containing block co-polymers having at least one polyanionic anchor region and at least one hydrophilic tail region, preferably wherein the polyanion-containing block co-polymer is a linear diblock and/or triblock co-polymer.

29. The composition according to claim 23, wherein said therapeutic nucleic acid construct encoding IL-12 comprises SEQ ID NO: 7.

30. A method for the localized expression of IL-12 in a mucosal tissue in a patient in need thereof, comprising administering to said patient a therapeutically-effective amount of a composition according to claim 23.

31. A method for inhibiting the growth of a mucosal cancer cell in a patient in need thereof, comprising administering to said patient a therapeutically effective amount of a composition according to claim 23.

32. A method for treating bladder cancer in a patient in need thereof, comprising administering to said patient a therapeutically effective amount of a composition according to claim 23.
